(19) United States
(12) Patent Application Publication
(10) Pub. No.: US 2026/0053676 A1
(43) Pub. Date: Feb. 26, 2026

(54) INTERFACING STRUCTURE FOR EYEWEAR

(71) Applicant: RESMED PTY LTD, Bella Vista, New South Wales (AU)

(72) Inventors: Ashley Taylor CONEN, Sydney (AU); Aaron Samuel DAVIDSON, Sydney (AU); Memduh GUNEY, Sydney (AU); Gregory Kendall KNIGHT, Sydney (AU); Ian Andrew LAW, Sydney (AU); Rupert Christian SCHEINER, Sydney (AU); Hugh Francis Stewart THOMAS, Sydney (AU); Albert Jack Greenwood WOFFENDEN, Sydney (AU); Nishant RANDHAWA, Sydney (AU)

(21) Appl. No.: 19/106,119

(22) PCT Filed: Aug. 25, 2023
(86) PCT No.: PCT/AU2023/050829

§ 371 Date: Feb. 24, 2025

(30) Foreign Application Priority Data
Aug. 25, 2022 (AU) 2022902442
Feb. 3, 2023 (AU) 2023900261
Feb. 28, 2023 (AU) 2023900509
Apr. 14, 2023 (AU) 2023901111

(51) Int. Cl. A61F 9/02

(52) U.S. Cl. CPC A61F 9/026; A61F 9/027

(57) ABSTRACT

An interfacing structure is connected, directly or indirectly, to a lens of eyewear, such as a goggle. The interfacing structure is constructed and arranged to be in opposing relation with a user's face in use. The interfacing structure includes a compliance portion comprising a support flange around a periphery of an eye region of the user's face, and a cushion support flange extending from the support flange, the support flange being flexible and resilient. The interfacing structure further includes a cushion configured to engage the user's face in use, the cushion provided to the cushion support flange.

### 1 CROSS-REFERENCE TO RELATED APPLICATIONS

This application claims the benefit of, and priority to, Australian Provisional Patent Application No. 2022902442 filed on 25 Aug. 2022, Australian Provisional Patent Application No. 2023900261 filed on 3 Feb. 2023, Australian Provisional Patent Application No. 2023900509 filed on 28 Feb. 2023, and Australian Provisional Patent Application No. 2023901111 filed on 14 Apr. 2023, each of which are hereby incorporated by reference herein in its entirety.

### 2 BACKGROUND OF THE TECHNOLOGY

### 2.1 Field of the Technology

The present technology relates to interfacing structures for eyewear. More particularly, the present technology relates to interfacing structures for a goggle.

### 2.2 Description of the Related Art

A goggle is a type of eyewear that surrounds a user's eyes and covers a portion of the face of the user. This type of eyewear is usually designed for activities that require increased protection of the wearer's eyes and face such as outdoor sports (e.g. snow sports), racing (e.g. motorcycle racing, or bicycle racing), projectile protection (e.g. paintball, airsoft or ballistic applications), safety applications (e.g. safety goggles), or general protection from harsh environmental conditions (e.g. harsh conditions such as in snow or desert environments).

Goggles typically include a lens, a cushion configured to contact a face of a user when the goggle is being worn, and a strap to hold the goggle in place on the user's head. Numerous modifications have been made to eyewear and goggle products to make these products more comfortable for the wearer.

### 3 BRIEF SUMMARY OF THE TECHNOLOGY

The present technology is directed towards providing eyewear having one or more of improved comfort, cost, efficacy, ease of use and manufacturability.

A first aspect of the present technology relates to an interfacing structure connected, directly or indirectly, to a lens of eyewear.

Another aspect of the present technology relates to eyewear comprising at least one lens, and an interfacing structure connected, directly or indirectly to the at least one lens.

In examples the eyewear is a goggle. In examples the goggle comprises a single lens.

In examples the eyewear comprises a positioning and stabilising structure structured and arranged to hold the lens and interfacing structure in an operable position on the user's head in use. In examples the positioning and stabilising structure is provided to the interfacing structure. In examples the positioning and stabilising structure is provided to the lens. In examples the positioning and stabilising structure is provided to an intermediary component between the interfacing structure and the lens.

A further aspect of the present technology relates to an interfacing structure connected, directly or indirectly, to a lens of eyewear, the interfacing structure constructed and arranged to be in opposing relation with the user's face in use, the interfacing structure comprising:
a face engaging portion comprising a face engaging flange provided around a periphery of an eye region of the user's face and configured to engage the user's face in use, the face engaging flange being flexible and resilient; and
a cushion at least partially covered by the face engaging flange.

In examples:
The face engaging portion is connected to the lens;
The goggles comprise a lens frame configured to support the lens;
The face engaging portion is connected to the lens frame;
The goggles comprise a chassis portion, the chassis portion being stiffer than the face engaging portion, the face engaging portion being attached to and supported by the chassis portion;
The chassis portion is connected to the lens frame; and/or
The chassis portion is connected to the lens.

In examples, the cushion is made of a foam material.

In examples, the face engaging flange is formed from an elastomeric material.

In examples the face engaging flange extends from a radially outward position to a radially inward position. In examples the face engaging flange curves over at least a portion of the cushion from the radially outward position.

In examples the cushion surrounds the eye region of the user's face. In examples the cushion includes a nasal portion extending over the user's nose in use. In examples the cushion is integrally formed.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient; and
a cushion configured to engage the user's face in use, the cushion provided to the cushion support flange.

In examples:
The compliance portion is connected to the lens;
The compliance portion is connected to a posterior surface of the lens;
The compliance portion is connected to a peripheral edge of the lens;
The goggles comprise a chassis portion, the chassis portion being stiffer than the compliance portion, the compliance portion being attached to and supported by the chassis portion;
The chassis portion is connected to the lens.

In examples, the cushion is made of a foam material. In example the foam material is a 3D foam. In examples a textile cover may be provided to the cushion. In examples the cushion may comprise a fleece and/or relatively high thickness textile layer.

In examples, the compliance portion is formed from an elastomeric material.

In examples the support flange extends from a radially inward position at the lens to a radially outward position at the cushion support flange. In alternative examples the support flange extends from a radially outward position at the lens to a radially inward position at the cushion support flange.

In examples the support flange connects to a radially outwards edge of the cushion support flange. In alternative examples the support flange connects to the cushion support flange at an intermediary position between edges of the cushion support flange. In examples the cushion overlaps the cushion support flange.

In examples the support flange comprises a thickened portion, wherein the cross-sectional thickness of the thickened portion is greater than at least one other portion of the support flange. In examples the thickened portion is proximal the lens.

In examples the interfacing structure comprises a nasal region. In examples, in the nasal region:
a posterior portion of the cushion at the nasal region is rounded;
the support flange comprises a thickened portion proximal the lens;
the support flange connects to the cushion support flange at an intermediary position between edges of the cushion support flange, wherein the radially inward edge of the cushion support flange is canted towards centreline;
the support flange is provided to a radially inward edge of the cushion support flange, and the compliance portion is configured such that the radially outward edge of the cushion support flange flexes radially outwardly;
the support flange is provided to a radially outward edge of the cushion support flange, and the radially inward edge of the cushion support flange is pre-loaded towards centreline;
the interfacing structure comprises an anterior nasal flange portion, the anterior nasal flange portion comprises a first portion proximal the sides of the user's nose in use, and a second portion proximal the ala crest, wherein the cross-sectional thickness of the first portion is greater than that of the second portion.

In examples the lens extends radially outward in at least a lateral direction beyond the interfacing structure.

In examples, the compliance portion in a nasal portion of the interfacing structure comprises an adaptation portion between the lens and a posterior nasal contact portion of the compliance portion. In examples the adaptation portion has a concave cross-section facing in the inferior direction.

In examples the compliance portion in the nasal portion of the interfacing structure comprises an intermediary portion between an adaptation portion and a posterior nasal contact portion of the compliance portion. In examples the adaptation portion has a concave cross-section facing in a radially outwards direction. In examples, the cross-sectional thickness of the intermediary portion is greater than that of the adaptation portion. In examples the posterior nasal contact portion curves in a radially inward direction from the intermediary portion. In examples the compliance portion in the nasal portion of the interfacing structure comprises lug portions extending laterally from the intermediary portion. In examples the lug portions are provided at an inferior position, proximal the transition between the nasal portion and the cheek portions. In examples a support member made of a more rigid material than the compliance portion comprises a bridging portion and lug locating features disposed at each end of the bridging portion. In examples the bridging portion spans over the nasal portion, against a lens connection portion. In examples the lug locating features secure to the lug portions, leaving an unconstrained portion of the lug portion between the lug locating features and the intermediary portion.

In examples, the support flange of the compliance portion at the forehead portion comprises an anterior support flange portion and a posterior support flange portion. In examples the posterior support flange portion extends in an inferior-posterior direction from the anterior support flange portion. In examples, the cushion support flange comprises a radially outward cushion support flange portion extending radially outward from the posterior support flange portion, and a radially inward cushion support flange portion extending radially inward from the posterior support flange portion.

In examples the compliance portion in the nasal portion of the interfacing structure comprises an adaptation portion between the lens connection portion and the posterior nasal contact portion of the compliance portion. Examples the adaptation portion comprises a bridge adaptation portion extending along the lens connection portion over the bridge of the user's nose in use, and lateral adaptation portions extending from the bridge adaptation portion in a posterior direction towards a posterior edge of the posterior nasal contact portion. In examples the adaptation portion comprises a curved transition adaptation portion between the bridge adaptation portion and the lateral adaptation portions. In examples, the bridge adaptation portion has a concave cross-section facing in the anterior-inferior direction, and the lateral adaptation portions have a concave cross-section facing in the posterior direction. In examples an anterior nasal contact portion extends in an anterior direction from the adaptation portion.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a face engaging flange around a periphery of an eye region of the user's face, wherein the face engaging flange is made of foam.

In examples the foam face engaging flange is curved in cross-section.

A further aspect of the present technology relates to eyewear, more particularly a goggle, comprising:
a lens;
an interfacing structure connected, directly or indirectly, to the lens, the interfacing structure constructed and arranged to be in opposing relation with the user's face in use, the interfacing structure comprising:
   a compliance portion comprising a support flange around a periphery of an eye region of the user's face, and a cushion support flange extending from the support flange, the support flange being flexible and resilient;
and a positioning and stabilising structure configured to generate a force to hold the interfacing structure to the user's face, wherein at least a portion of the positioning and stabilising structure is integral with the interfacing structure.

A further aspect of the present technology relates to a positioning and stabilising structure configured to be connected, directly or indirectly, to a lens of eyewear, comprising:
a pair of lateral strap portions configured to be secured, directly or indirectly, to the lens and lie on each side of the user's head in use;
a pair of rigidised portions connected to the pair of lateral strap portions;
a superior posterior strap portion configured to extend in a superior-posterior direction from the rigidised portions around the back of the user's head in use;
an inferior posterior strap portion configured to extend in a posterior direction from the rigidised portions, inferior to the superior posterior strap portion, around the back of the user's head in use.

In examples the rigidised portions are made of a form holding material. In examples the rigidised portions are made of a textile/foam laminate. In examples the rigidised portions are configured to extend in a superior-posterior direction from the lateral strap portions. In examples, the length of one or both of the superior posterior strap portion and the inferior posterior strap portion may be adjustable.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
wherein the support flange comprises at least one hydrophilic vent portion comprising hydrophilic textile material between an interior space defined by the eyewear and the user in use, and an exterior.

In examples a foam vent feature may be provided on an external surface of the hydrophilic vent portions.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
wherein the support flange comprises a plurality of vents; and
a cover portion provided to radially outward surfaces of the support flange, covering the plurality of vents, wherein the cover portion is hydrophobic.

In examples the vents are geometric shapes. In examples the vents are circular. In examples the vents are linear shapes. In examples, the vents are disposed towards an anterior of the compliance portion. In examples the size of the vents graduates from a size greatest proximal a centreline of the compliance portion, reducing in size laterally away from the centreline. In examples vents are provided proximal a transition between lateral portions and cheek portions of the compliance portion. In examples the cover portion extends continuously over all of the vents. In examples a plurality of discrete cover portions may be provided to different groups of vents.

In examples the compliance portion comprises a reinforcement portion extending between laterally disposed points on an anterior edge of the compliance portion, offset in a posterior direction from the anterior edge when intersecting the centreline. In examples, the majority of vents in the forehead portion are provided within the bounds of the anterior edge and the reinforcement portion.

In examples, the compliance portion comprises an anterior radially outward flange portion extending from the lens connection portion. In examples, the anterior radially outward flange portion is provided in the forehead portion and lateral portions of the compliance portion. In examples vents may be provided in the compliance portion in locations shielded by the anterior radially outward flange portion. In examples the vents may be provided proximal a transition between the lateral portions and the cheek portions.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
wherein the support flange comprises a plurality of vents; and
at least one vent valve provided to at least one of the plurality of vents, wherein the at least one vent valve comprises a flap portion structured and arranged to regulate flow through the at least one of the plurality of vents.

In examples the flap portion is provided on a radially outward facing surface of the support flange. In examples the flap portion is connected to the compliance portion on an anterior side of the at least one of the plurality of vents, and extends over the at least one of the plurality of vents in the anterior to posterior direction. In examples, the flap portion is biased or pre-loaded to an open position. In examples, in the open position the flap portion extends at an acute angle relative to the portion of the compliance portion over which it extends. In examples the flap portion is configured to close when airflow in the anterior to posterior direction is above a threshold. In examples the flap portion is configured to close to an extent proportional to the relative speed of airflow in the anterior to posterior direction.

In examples the vent valve is configured to maintain a gap between the flap portion and the compliance portion when the flap portion is in a closed position. In examples one or more vent valve spacer portions are provided on a posterior side of the vents.

In examples the flap portion may include at least one secondary vent which at least partially overlaps at least one of the plurality of vents when the flap portion is in a closed position. In examples an area of the at least one secondary vent may be less than an area of the at least one of the plurality of vents.

In examples the at least one vent valve may be provided to one or more, but not all, of the plurality of vents in the compliance portion. In examples, a vent valve may be provided to vents proximal a transition between lateral portions and cheek portions of the compliance portion, not vents provided in a forehead portion of the compliance portion.

In examples, the flap portion may be made of a resilient and flexible material. In examples the flap portion may be made of an elastomer. In examples the elastomer may be silicone. In examples the flap portion may be integrally formed with the compliance portion. In examples the flap portion may be secured to the compliance portion.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
a chassis portion configured to secure the lens relative to the compliance portion, the chassis portion being stiffer than the compliance portion, wherein the compliance portion is provided to the chassis portion.

In examples, the chassis portion may be made of the same material as the compliance portion, but of different durometer. In examples, the chassis portion may be made of a different material to the compliance portion. In examples the chassis portion may be made of a non-elastomer plastic and the compliance portion may be made of an elastomer.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
an anterior chassis portion configured to secure the lens relative to the compliance portion, wherein the anterior chassis portion comprises a superior chassis portion and an inferior chassis portion,
wherein the interfacing structure is configured to allow resilient movement of the superior chassis portion relative to the inferior chassis portion in a superior-inferior direction.

In examples the interfacing structure comprises at least one chassis extension feature between the superior chassis portion and the inferior chassis portion. In examples, the at least one chassis extension feature comprises a portion of the flexible and resilient support flange interconnecting the superior chassis portion and an inferior chassis portion. In examples, the at least one chassis extension feature comprises at least one living hinge feature provided between the superior chassis portion and the inferior chassis portion. In examples the living hinge feature may comprise a concertina or a bellows structure.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
an anterior chassis portion configured to secure the lens relative to the compliance portion, wherein the support flange extends in a posterior direction from the anterior chassis portion,
wherein the anterior chassis portion and a superior portion of the support flange have a relatively greater stiffness than at least an inferior portion of the support flange.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
a cover portion extending between an anterior position, over radially outward surfaces of the support flange, and a radially outward portion of the cushion support flange, such that a gap is provided between the cover portion and the radially outward surfaces of the support flange.

In examples, the cushion support flange comprises a cover support flange extending in an anterior direction from the radially outward portion of the cushion support flange. In examples the cover support flange is provided in an intermediary position between the support flange and a distal end of the radially outward portion of the cushion support flange. In examples the cover portion is provided directly to the cover support flange.

In examples, the interfacing structure comprises an anterior cover locating feature configured to locate the cover portion at the anterior position. In examples, the anterior cover locating feature is provided in a chassis portion configured to secure the lens relative to the compliance portion. In examples, the anterior cover locating feature comprises a first radially outward surface leading to a radially inwardly angled transition surface, and a posterior facing surface extending in a radially outward direction to a second radially outward surface, wherein the second radially outward surface is elevated beyond the first radially outward surface.

In examples, the interfacing structure comprises a cover spacer feature configured to maintain the gap between the cover portion and the radially outward surfaces of the support flange. In examples the cover spacer feature comprises one or more anterior-posterior spacer features extending between an anterior position and an posterior position along the support flange, and projecting in a radially outward direction from the support flange. In examples the cover spacer feature comprises at least one lateral spacer feature between adjacent anterior-posterior spacer features.

In examples the cover spacer feature may be integral with the support flange.

In examples, the cover spacer feature may have a relatively high stiffness compared to the support flange. In examples, the cover spacer feature may be made of the same material as a chassis portion configured to secure the lens relative to the compliance portion.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
a cover portion extending over radially outward surfaces of the support flange, such that a gap is provided between the cover portion and the radially outward surfaces of the support flange, wherein the cover portion is configured to maintain a non-linear shape.

In examples, the cover portion may comprise a cover layer, and a shape holding layer. In examples the shape holding layer may be configured to provide a relatively stiff structure in comparison with the cover layer. For example, the cover layer may be made of a textile, and the shape holding layer may be made of a foam.

In examples, the cover portion may comprise a cover layer, and one or more discrete cover rigidiser features. The positioning of the one or more discrete cover rigidiser features is configured to provide greater stiffness to the cover layer in certain portions of the cover portion.

A further aspect of the present technology relates to an interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient,
wherein the support flange comprises a radially outward textile layer, and at least one rigidiser feature configured to increase stiffness of the textile layer.

Another aspect of one form of the present technology is an interface structure that is moulded or otherwise constructed with a perimeter shape which is complementary to that of an intended wearer.

An aspect of one form of the present technology is a method of manufacturing apparatus.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Eyewear

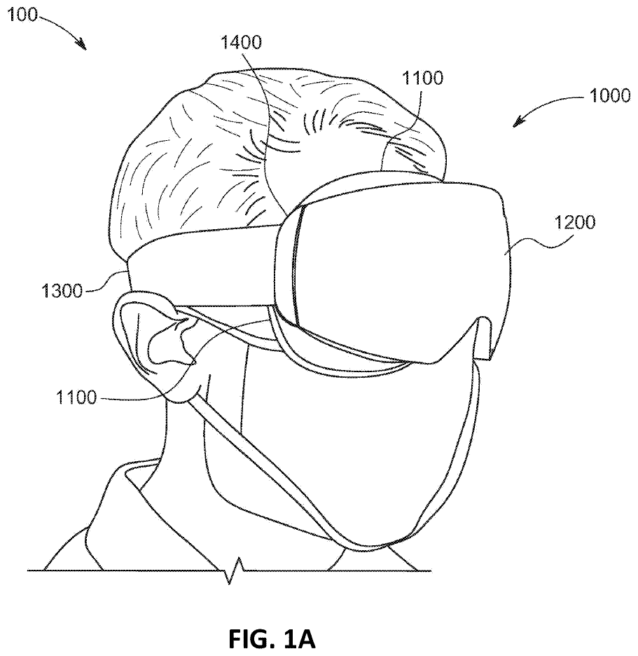

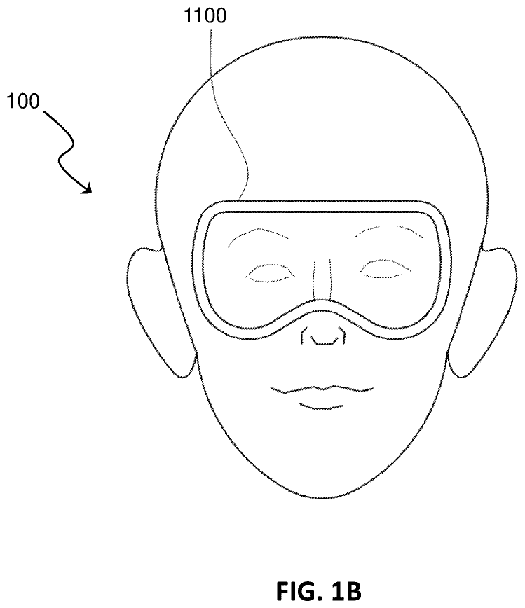

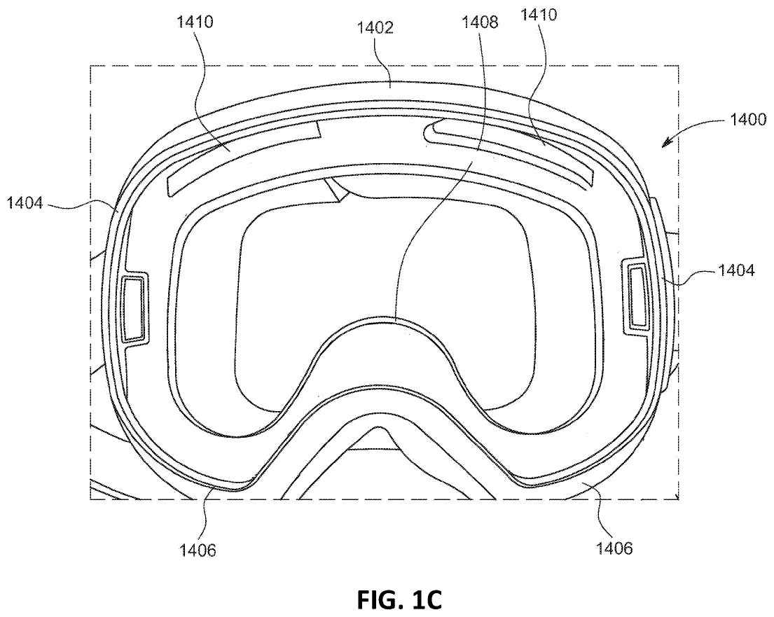

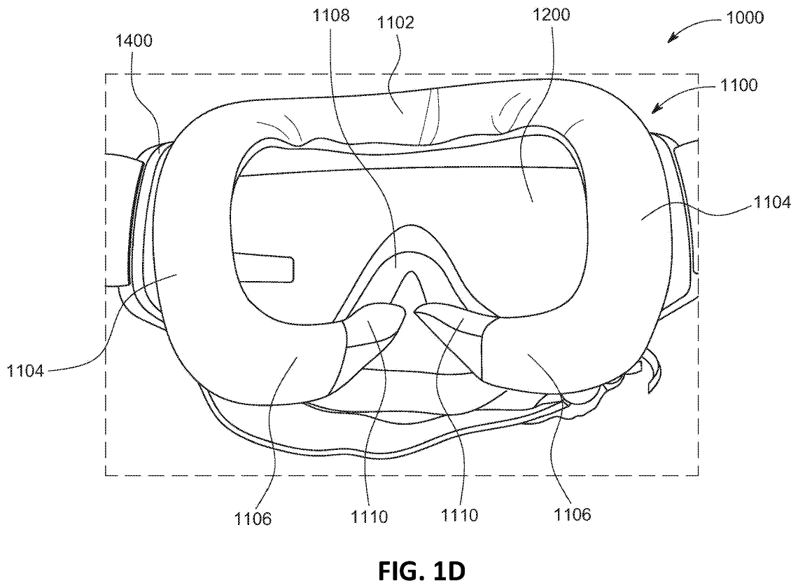

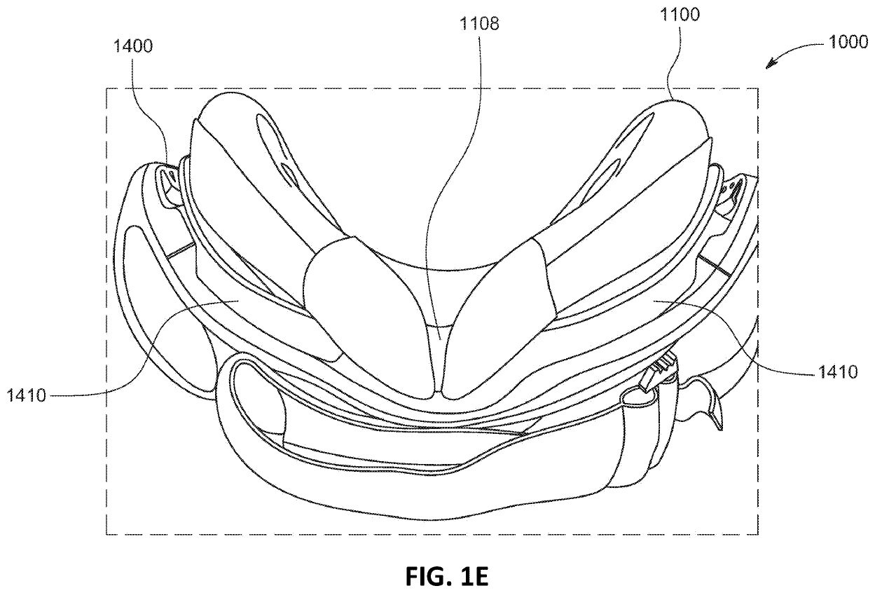

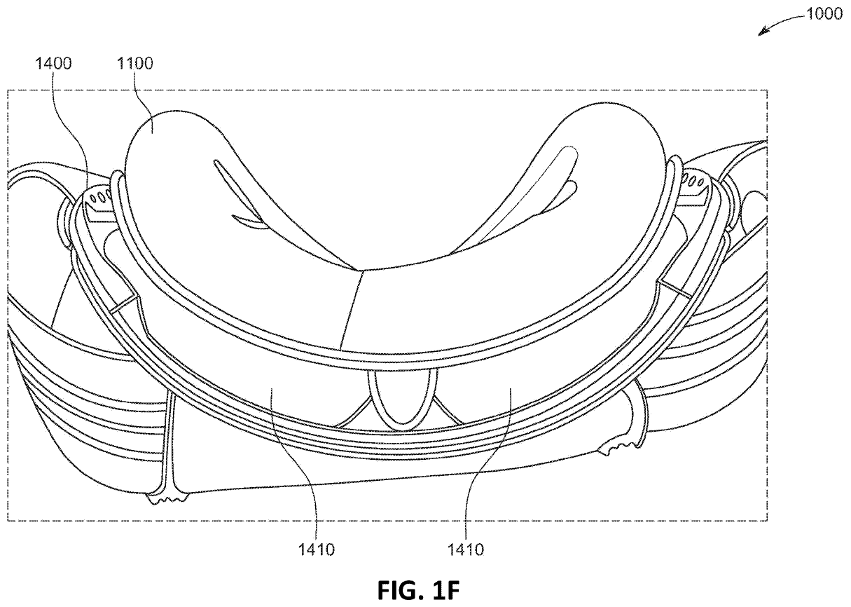

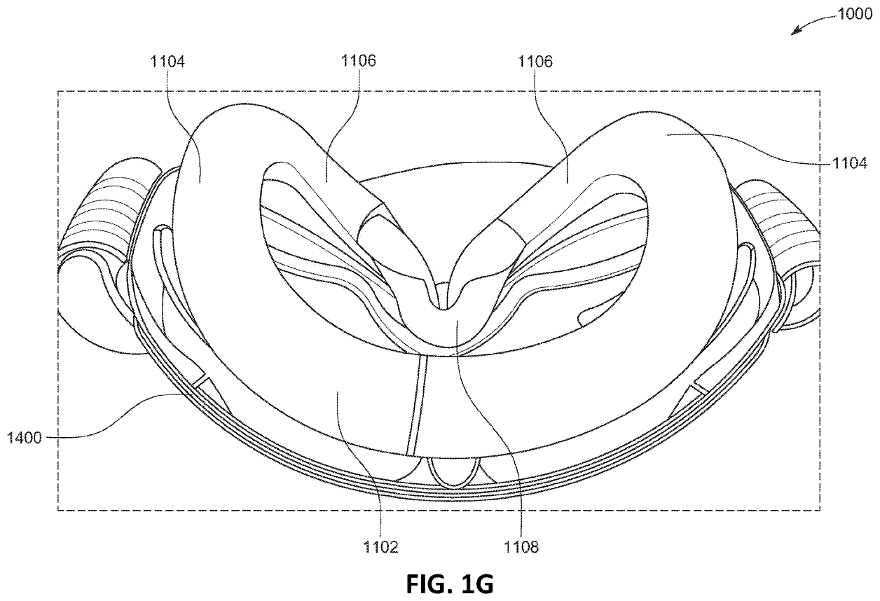

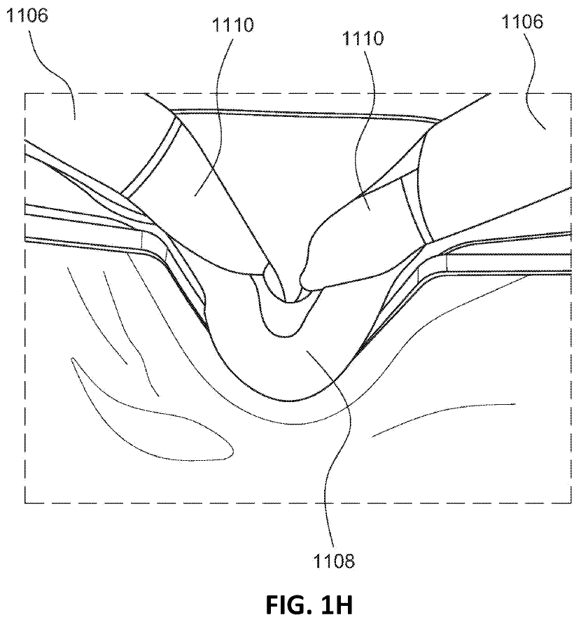

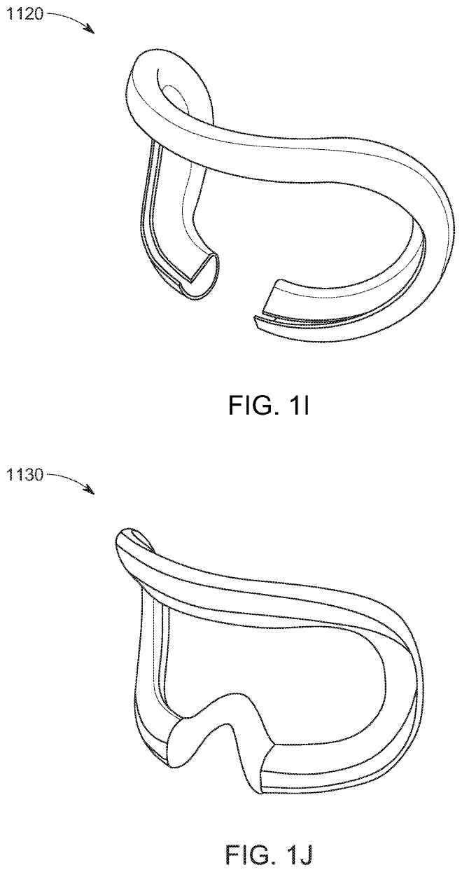

FIG. 1A shows a system including a user 100 wearing eyewear, in the form of a goggle 1000 comprising an interfacing structure according to one aspect of the present technology.

FIG. 1B shows a front view of a user's face, illustrating a location of the interfacing structure of the goggles, in use.

FIG. 1C shows a front view of a lens frame of the goggles.

FIG. 1D shows a rear view of the goggles.

FIG. 1E shows a bottom view of the goggles.

FIG. 1F shows a top view of the goggles.

FIG. 1G shows a rear perspective view of the goggles.

FIG. 1H shows a nasal region of the interfacing structure of the goggles.

FIG. 1I shows a perspective view of a face engaging flange of the interfacing structure of the goggles.

FIG. 1J shows a perspective view of a cushion of the interfacing structure of the goggles.

FIG. 1K is a cross-sectional view of the interfacing structure of the goggles.

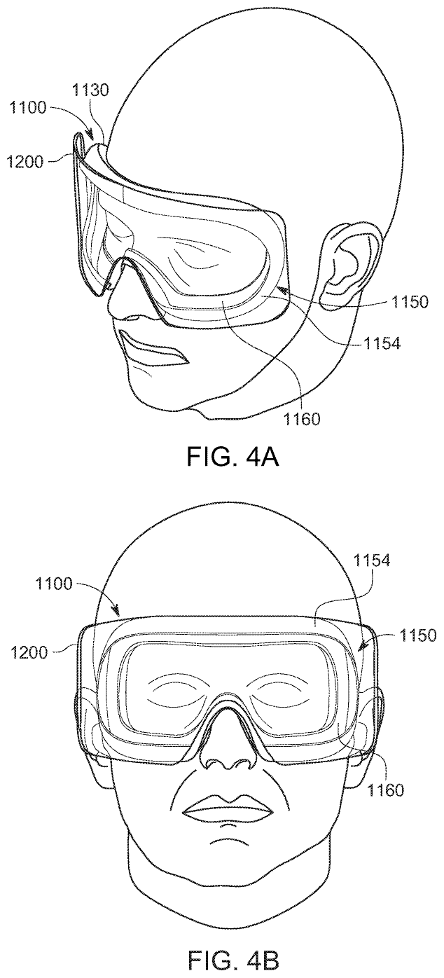

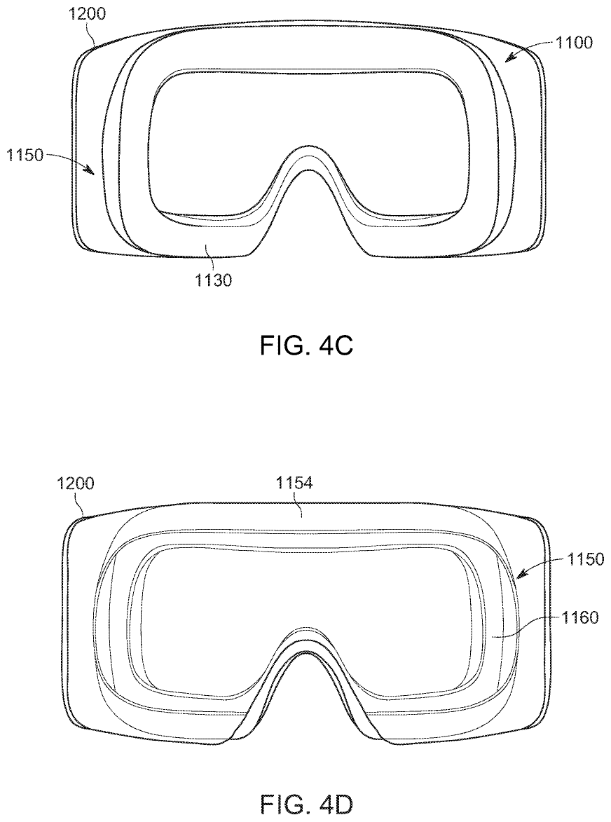

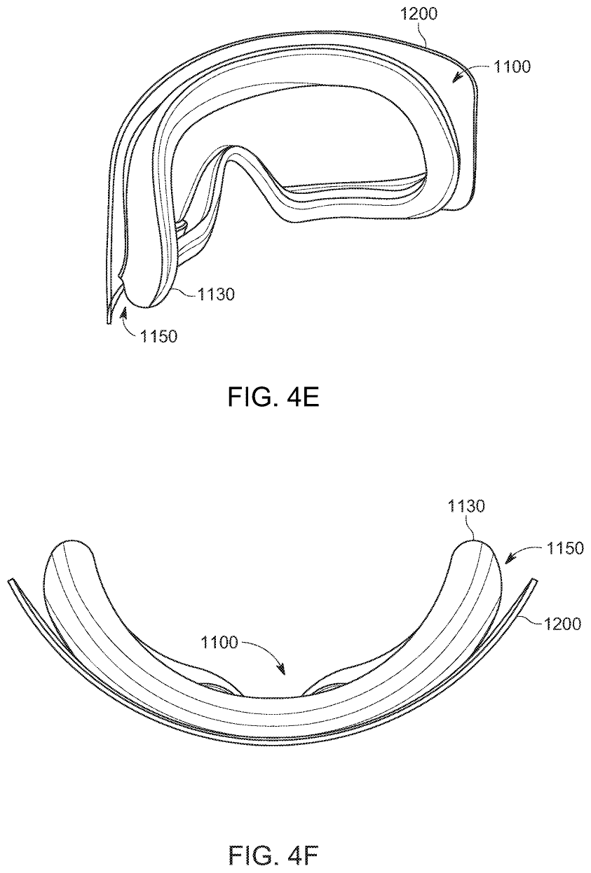

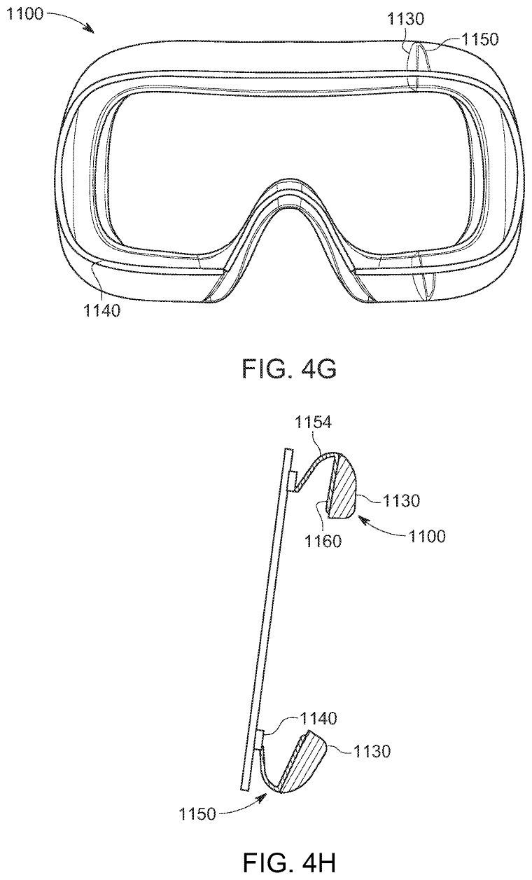

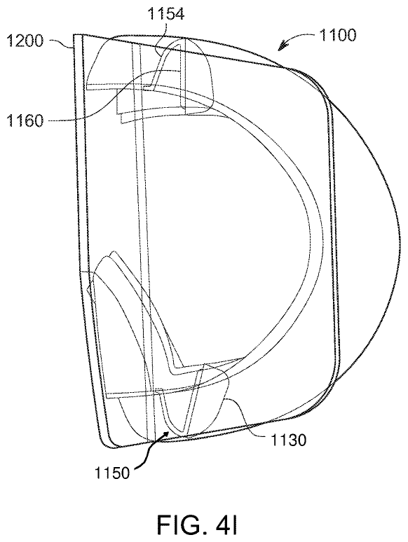

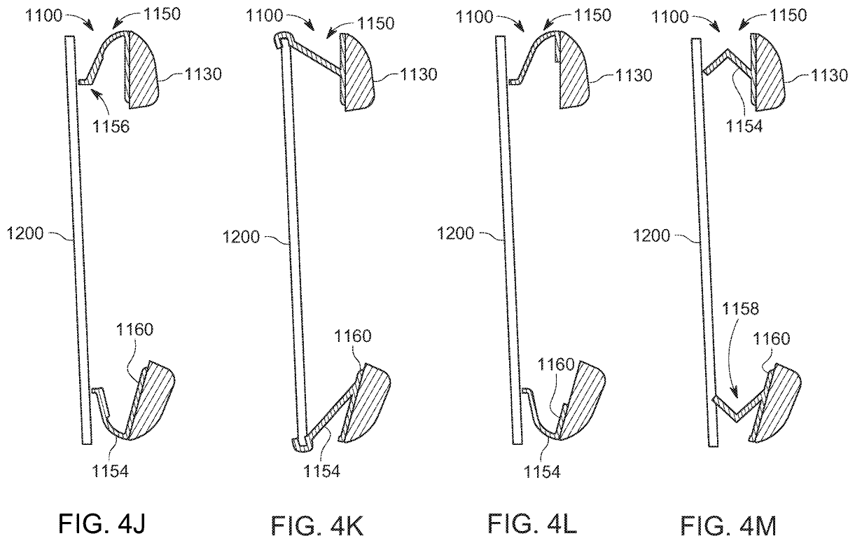

FIG. 4A and FIG. 4B show a user wearing goggles comprising an interfacing structure according to another aspect of the present technology.

FIG. 4C shows a rear view of the goggles.

FIG. 4D shows a front view of the goggles.

FIG. 4E shows a rear perspective view of the goggles.

FIG. 4F shows a top view of the goggles.

FIG. 4G shows a rear view of the goggles.

FIG. 4H and FIG. 4I show side cross-sectional views of the goggles.

FIG. 4J to FIG. 4M shows a side cross-sectional view of the goggles according to further examples.

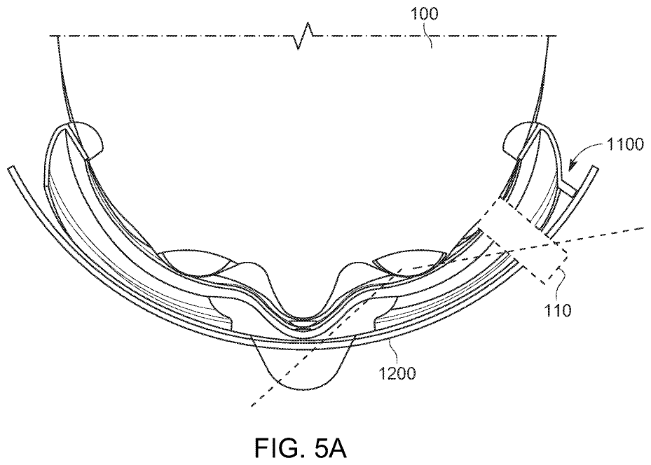

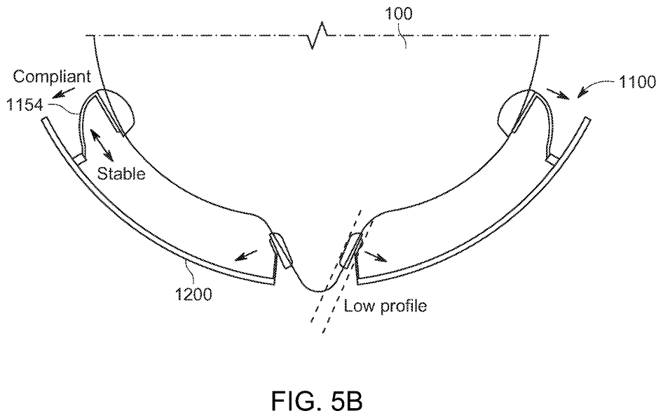

FIG. 5A shows a top cross-sectional view comparing field of view of goggles comprising an interfacing structure according to an aspect of the present technology with goggles having an interfacing structure of a typical construction.

FIG. 5B shows another top cross-sectional view of the goggles.

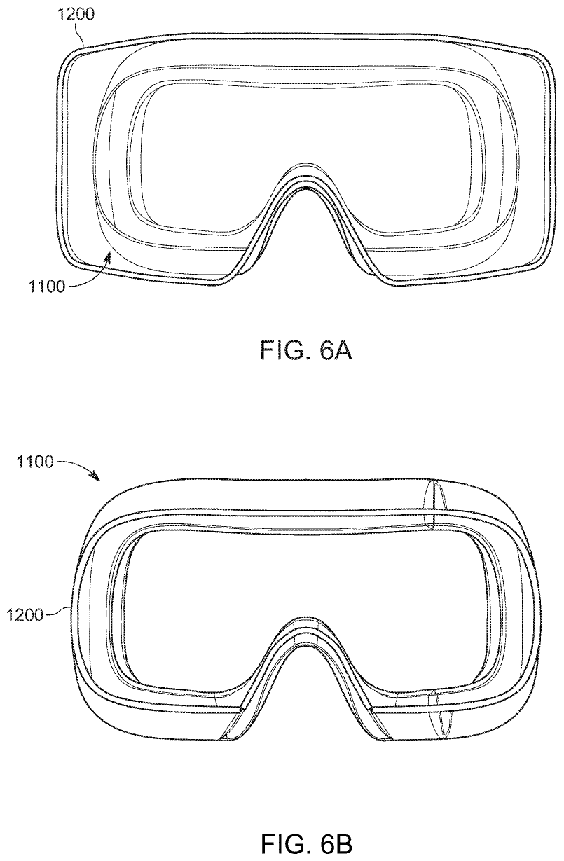

FIG. 6A is a front view of goggles having a lens extending laterally beyond the periphery of the interfacing structure.

FIG. 6B is a front view of goggles having a lens contained within the periphery of the interfacing structure.

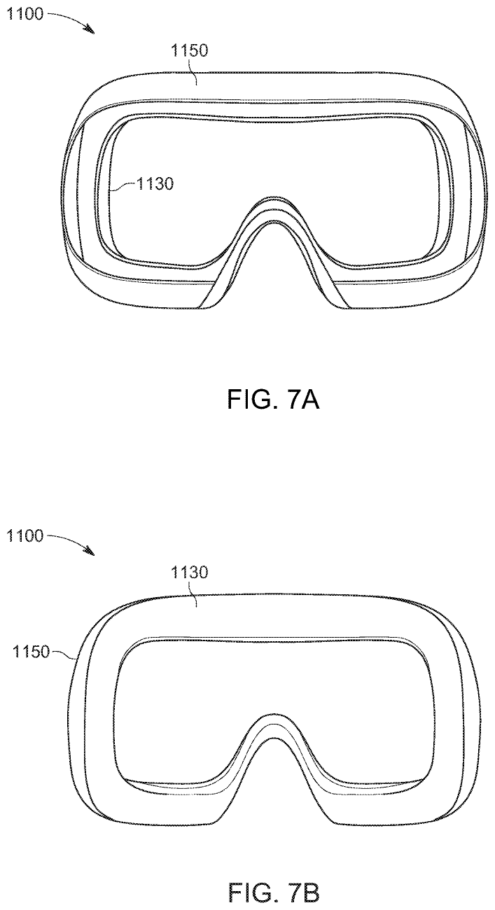

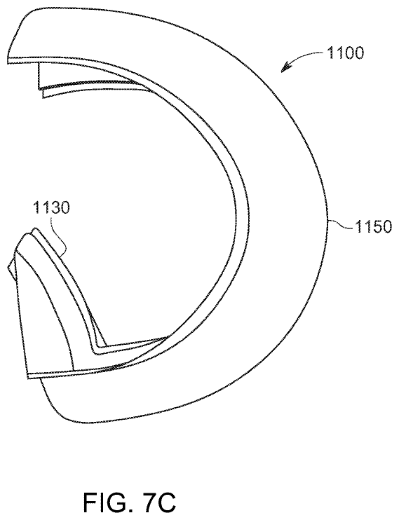

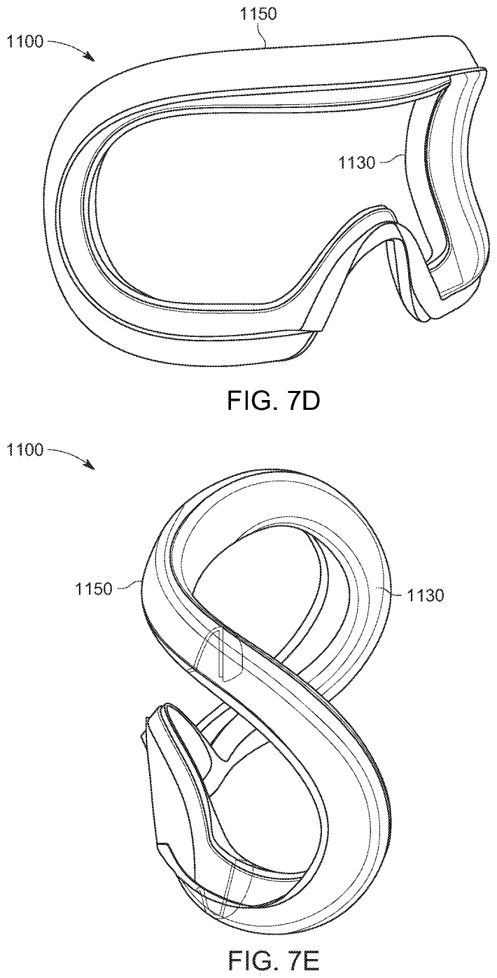

FIG. 7A is a front view of the interfacing structure according to an aspect of the present technology.

FIG. 7B is a rear view of the interfacing structure.

FIG. 7C is a side view of the interfacing structure.

FIG. 7D is a front perspective view of the interfacing structure.

FIG. 7E is a side perspective view of the interfacing structure.

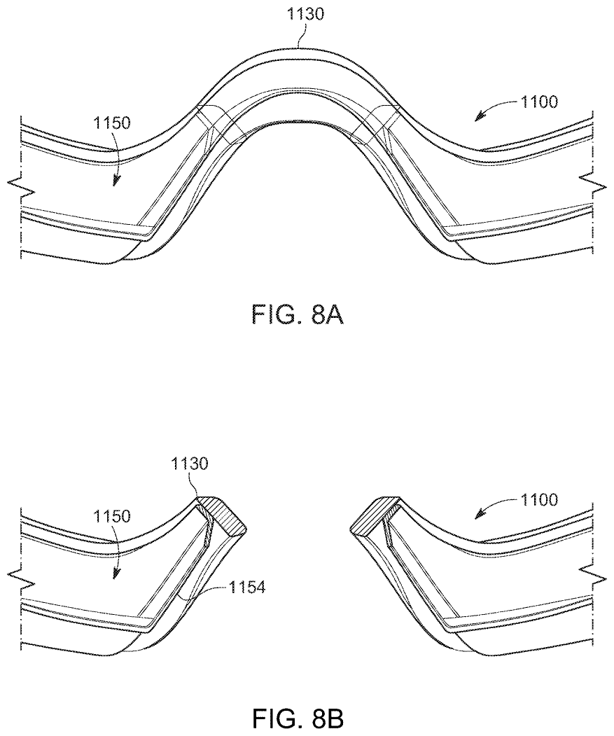

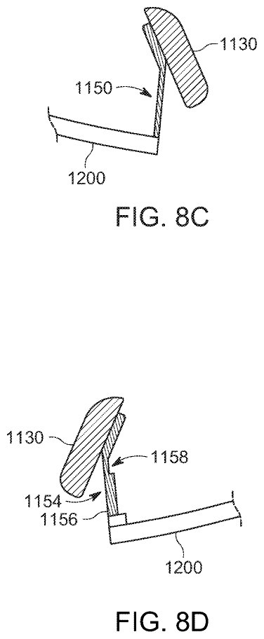

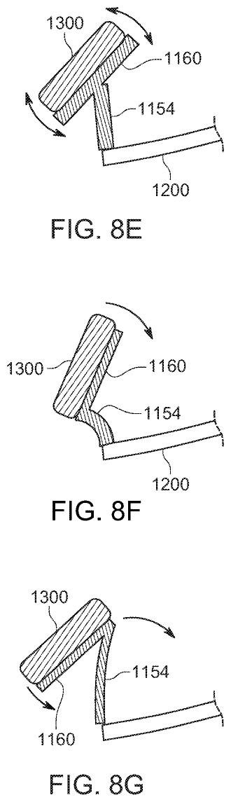

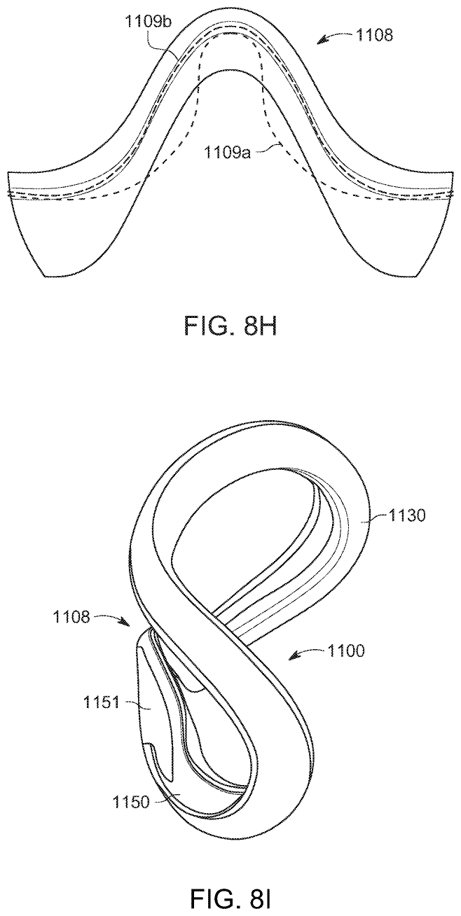

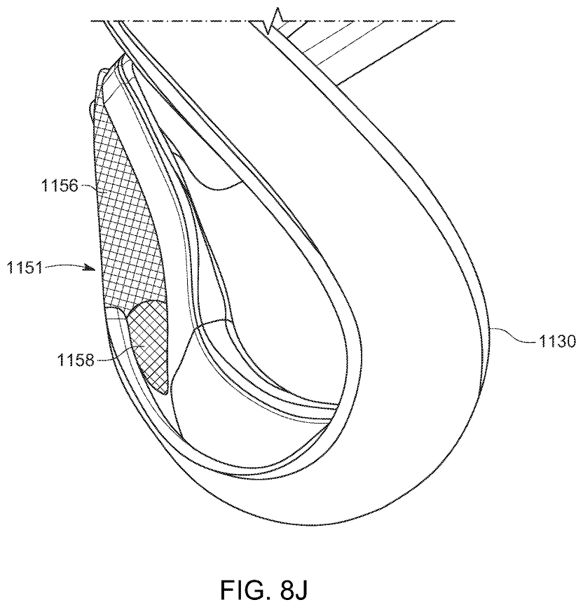

FIG. 8A is a front view of a nasal portion of the interfacing structure according to an aspect of the present technology.

FIG. 8B is a front view of the nasal portion of the interfacing structure, having a cutaway section to show the cross-section.

FIG. 8C is a cross-sectional view of the nasal portion of the interfacing structure.

FIG. 8D to FIG. 8G are cross-sectional view of alternative examples of the nasal portion of the interfacing structure.

FIG. 8H is a rear view of the nasal portion of the interfacing structure illustrating different profiles.

FIG. 8I is a side perspective view of the interfacing structure highlighting an anterior nasal flange portion.

FIG. 8J is an enlarged view of the anterior nasal flange portion.

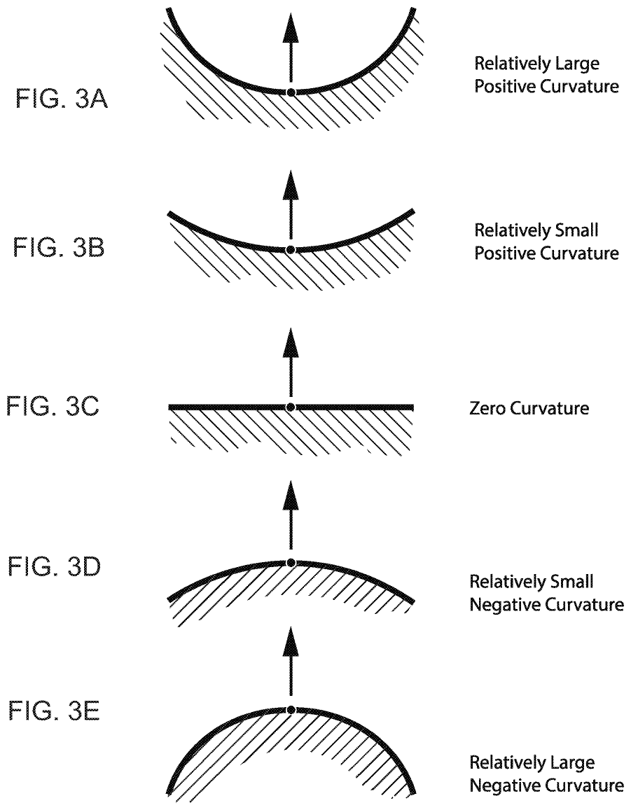

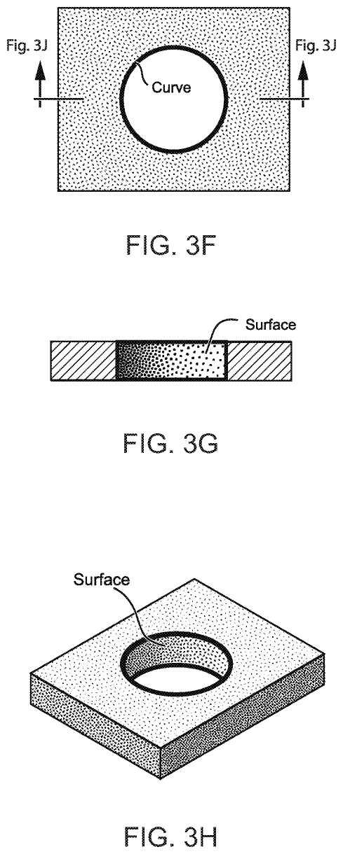

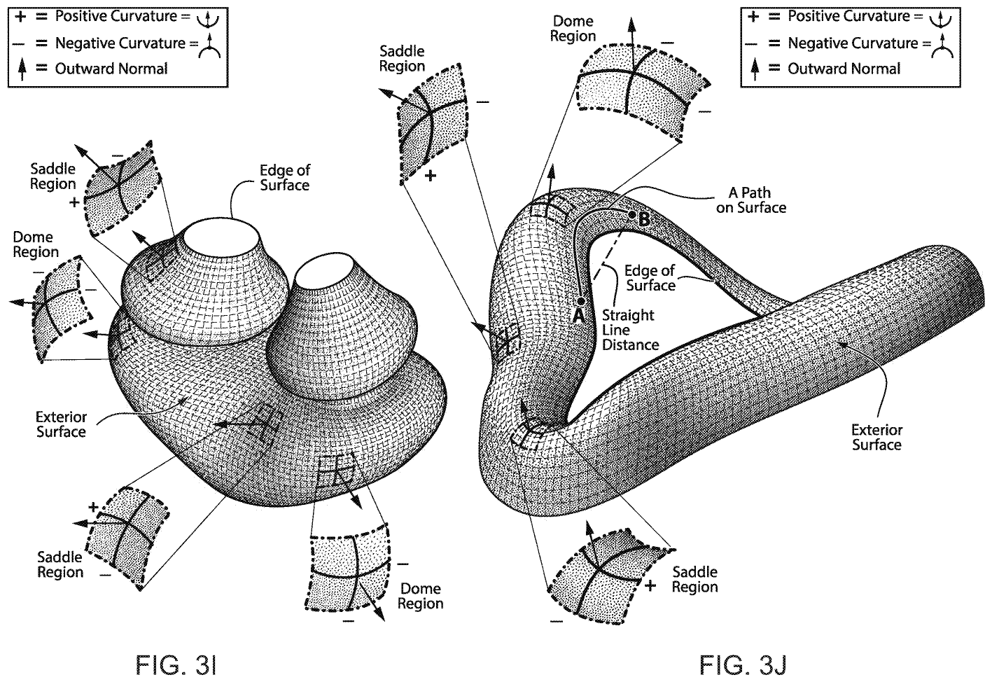

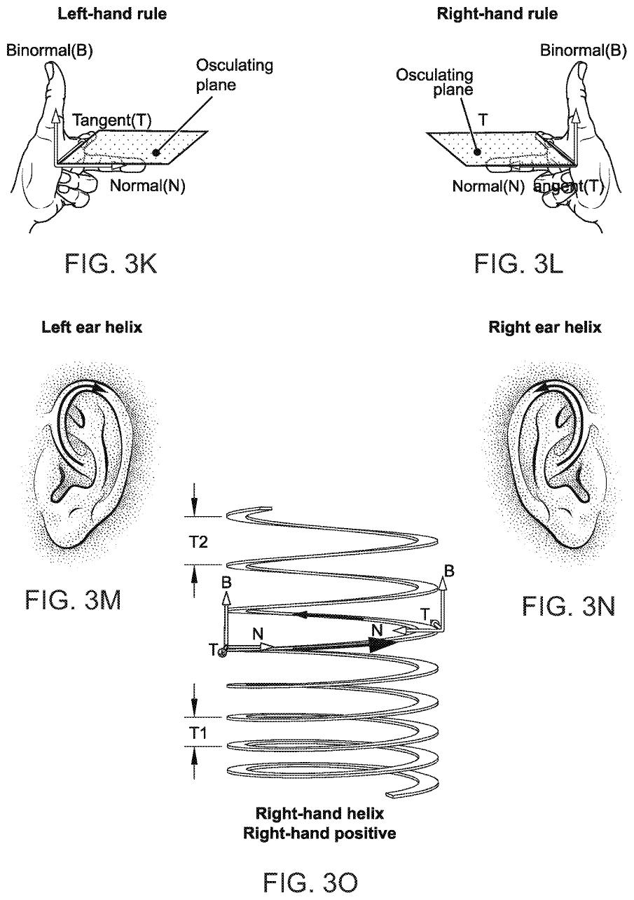

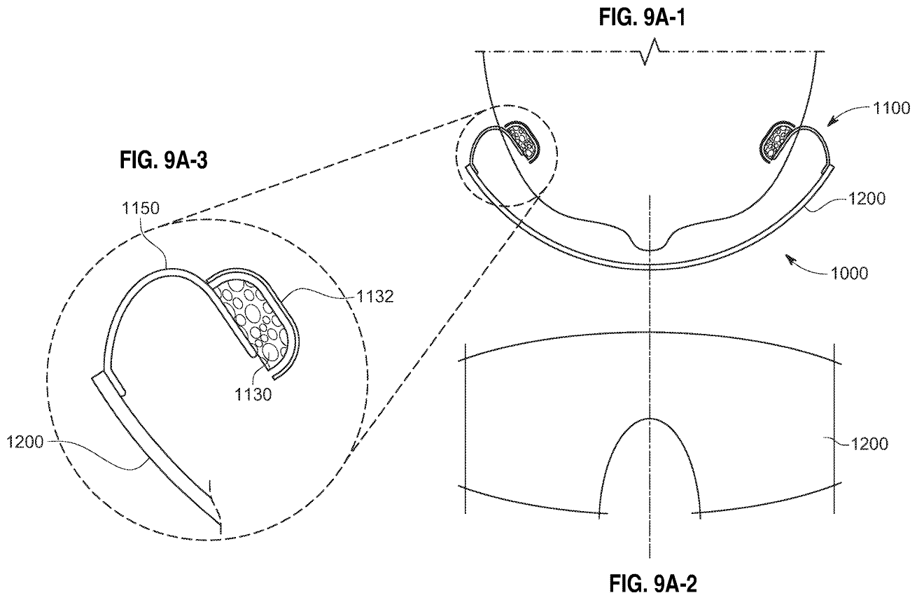

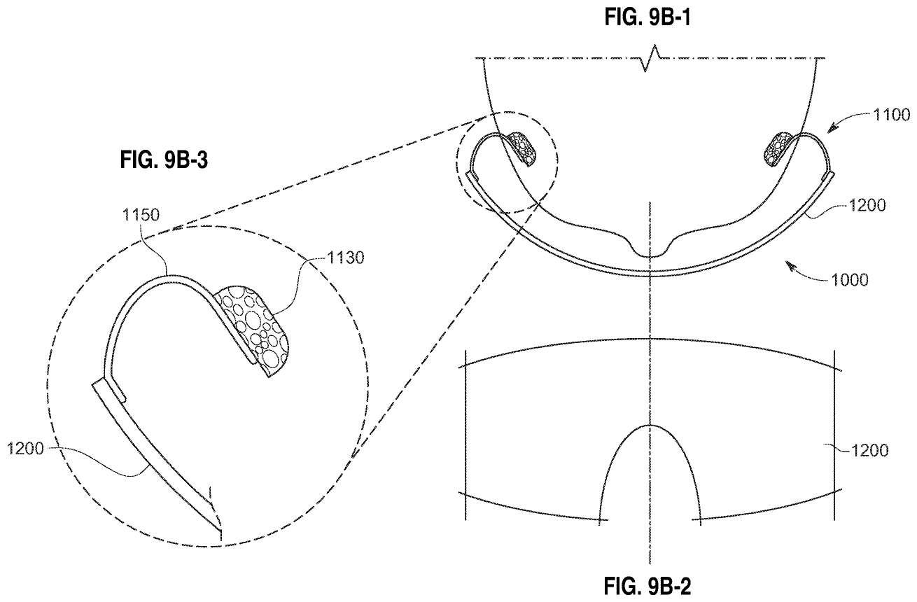

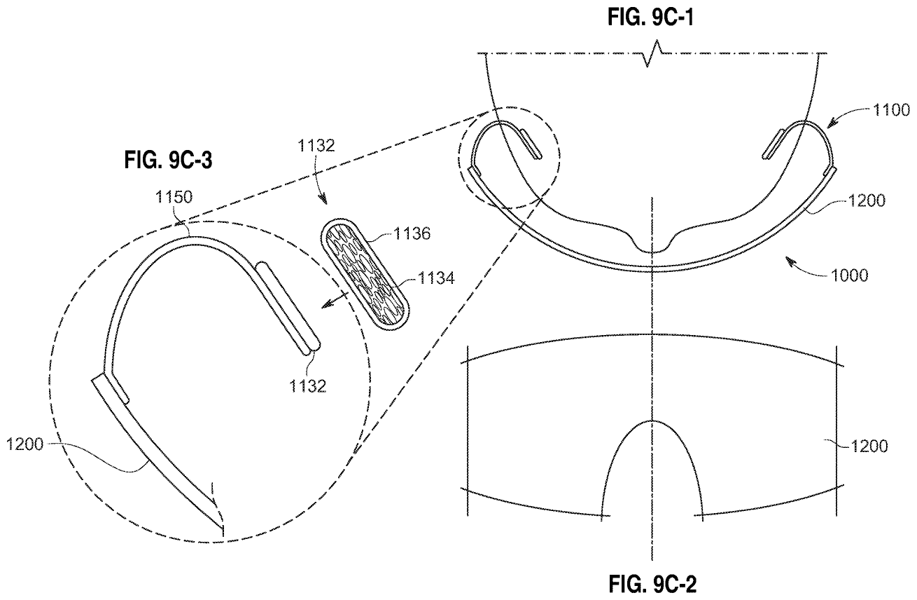

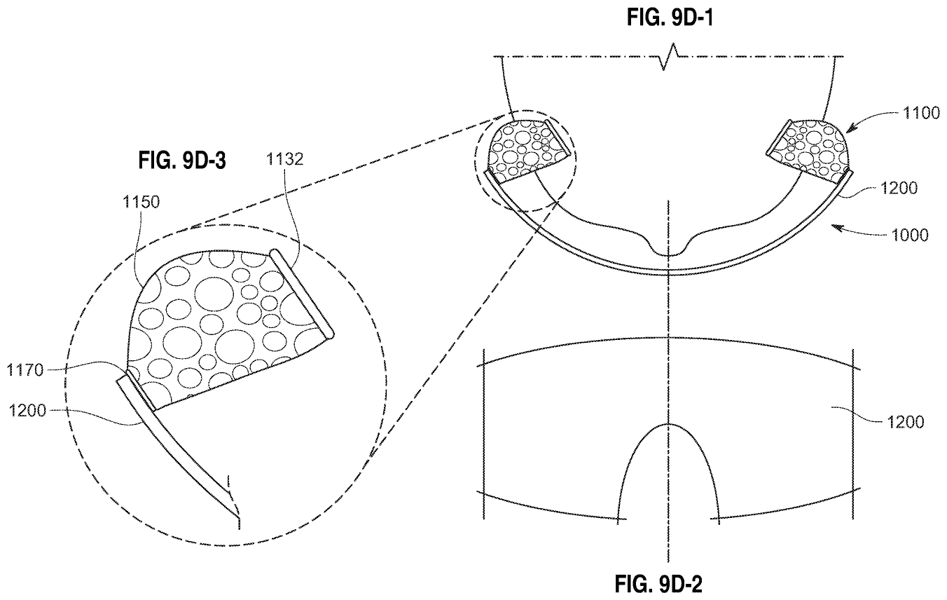

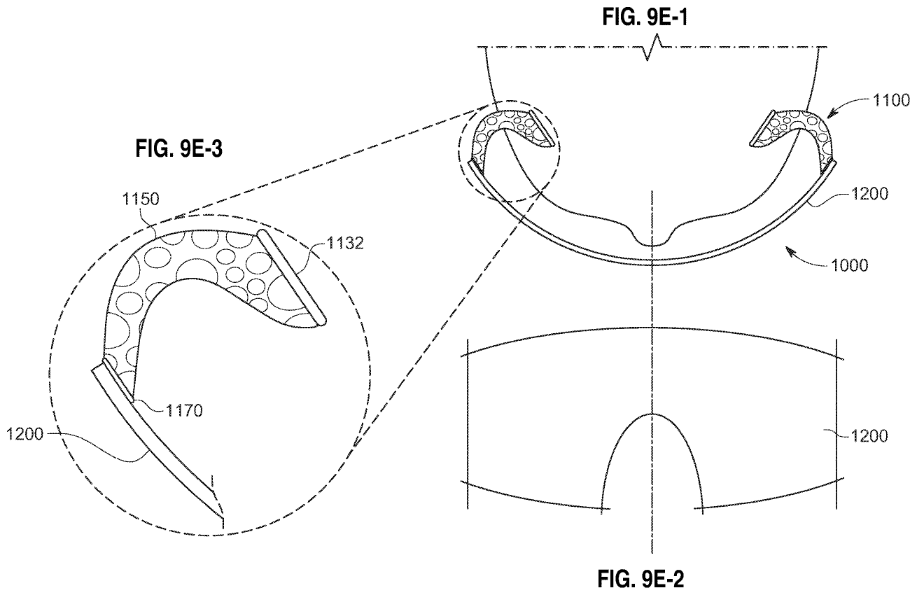

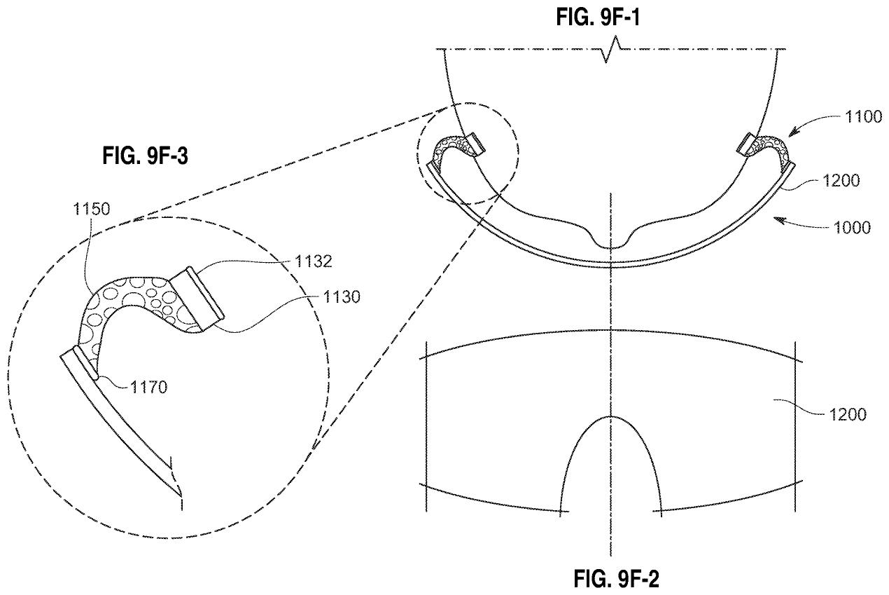

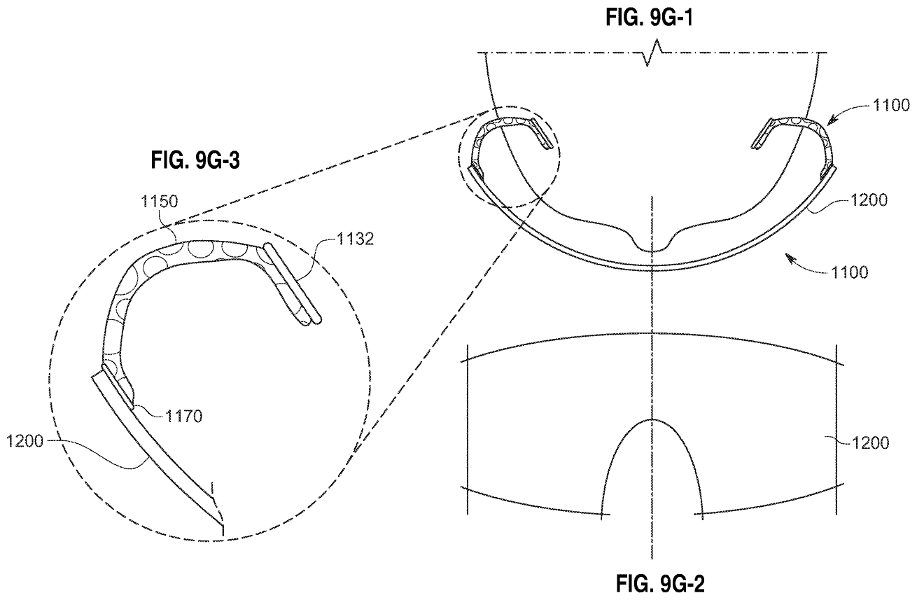

FIG. 9A to FIG. 9G-3 show alternative examples of an interfacing structure according to aspects of the present technology.

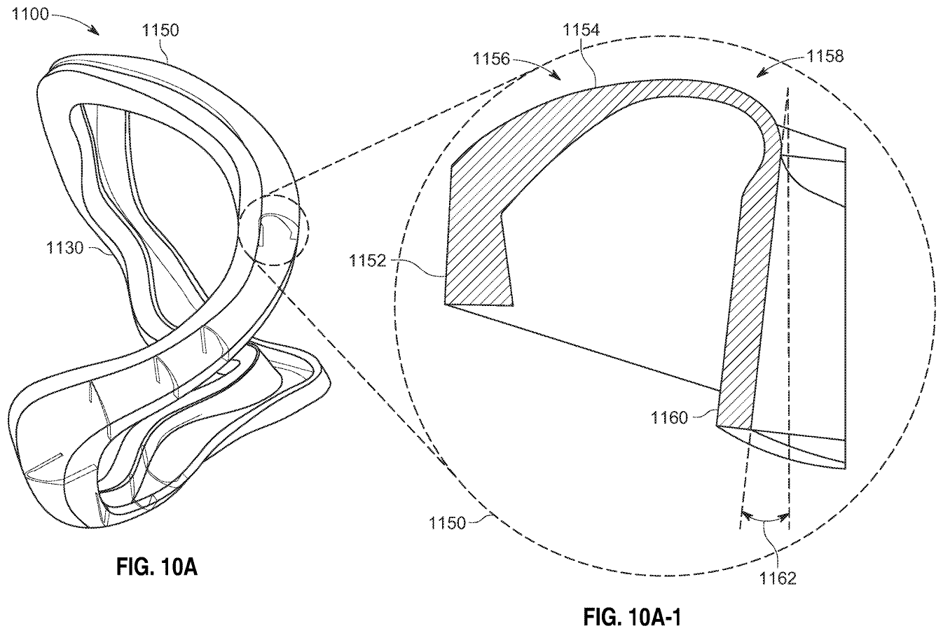

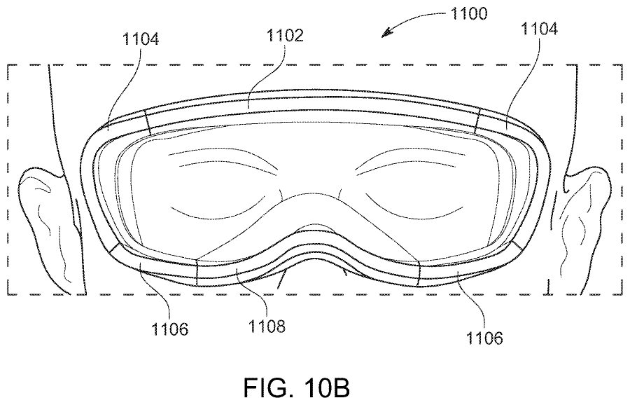

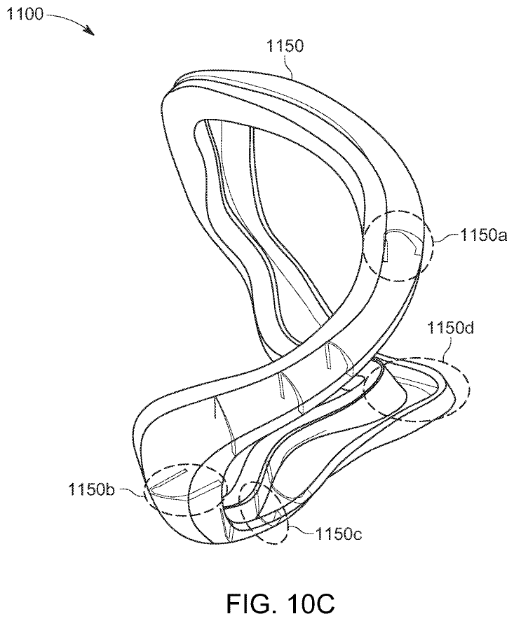

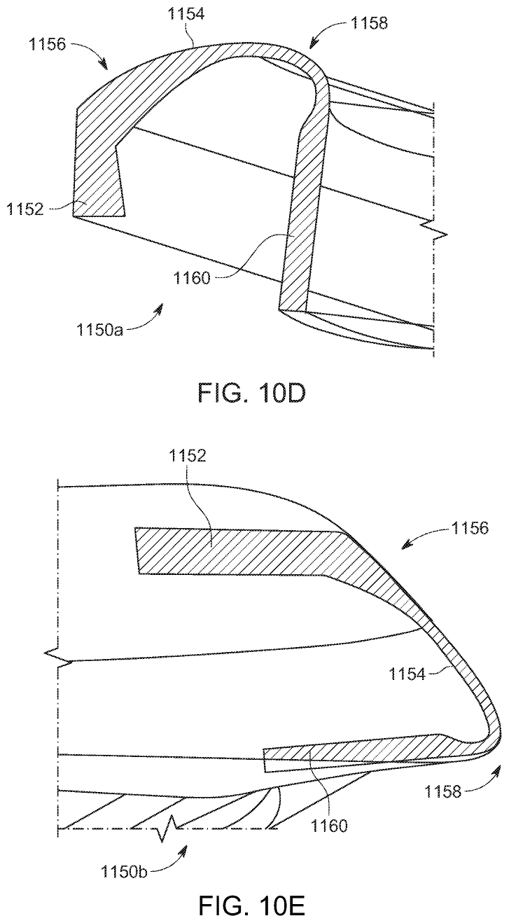

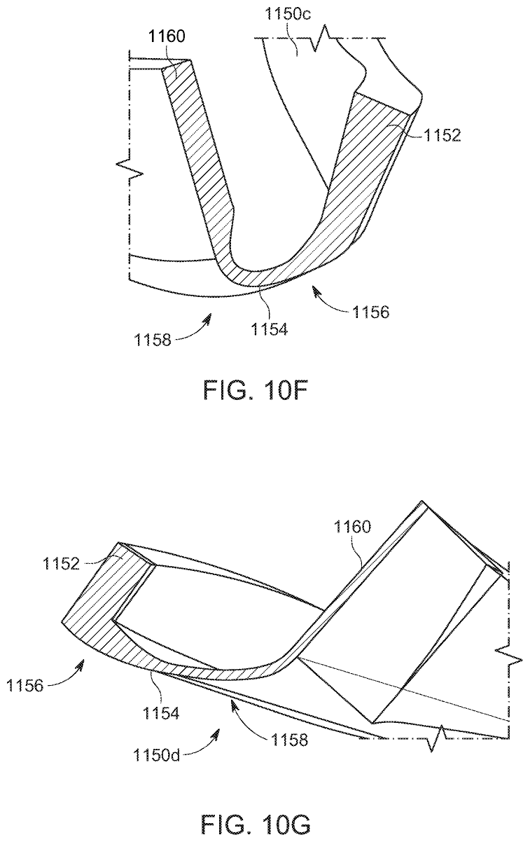

FIG. 10A to FIG. 10C show an exemplary interfacing structure according to an aspect of the present technology.

FIG. 10D to FIG. 10G are cross-sectional views of an exemplary compliance portion of the interfacing structure.

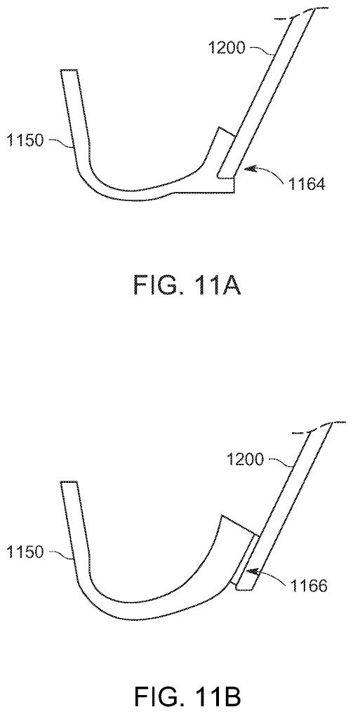

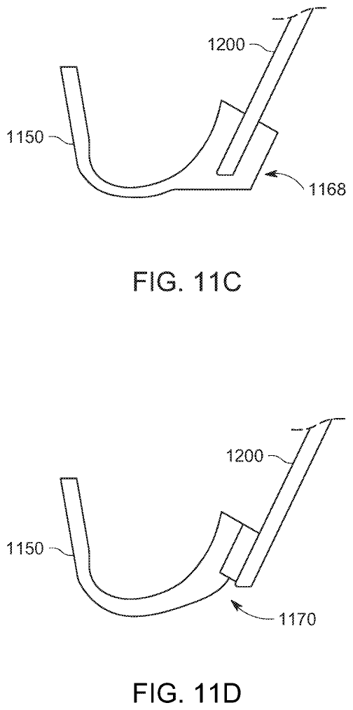

FIG. 11A to FIG. 11D are cross-sectional views showing exemplary means for securing a lens relative to an exemplary interfacing structure according to an aspect of the present technology.

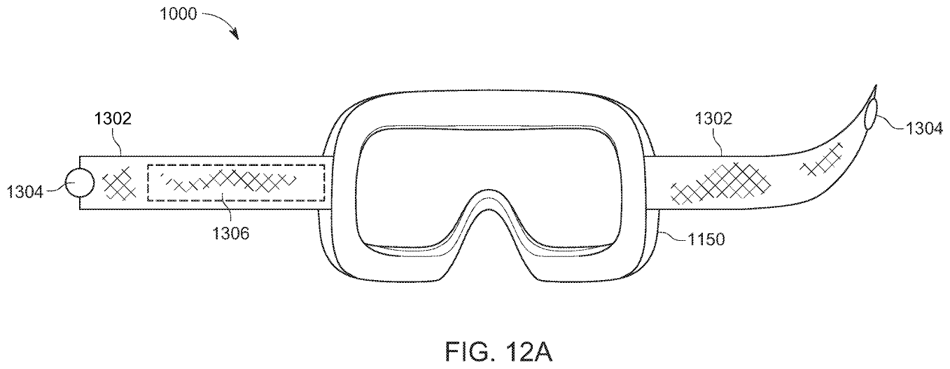

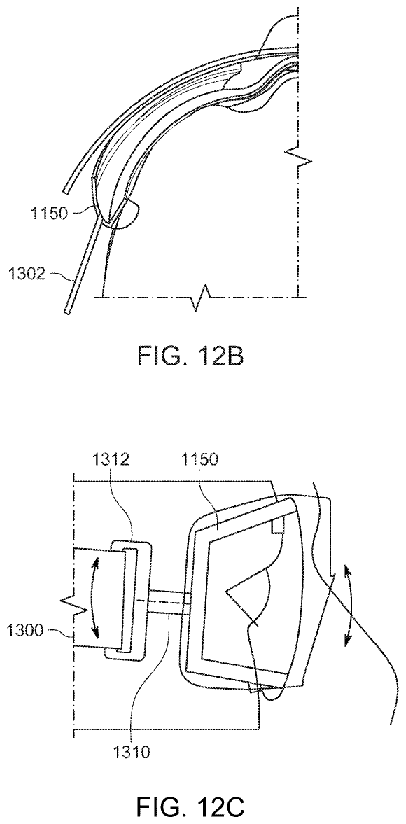

FIG. 12A to FIG. 12C show exemplary arrangements for a positioning and stabilising structure integrated with the interfacing structure according to an aspect of the present technology.

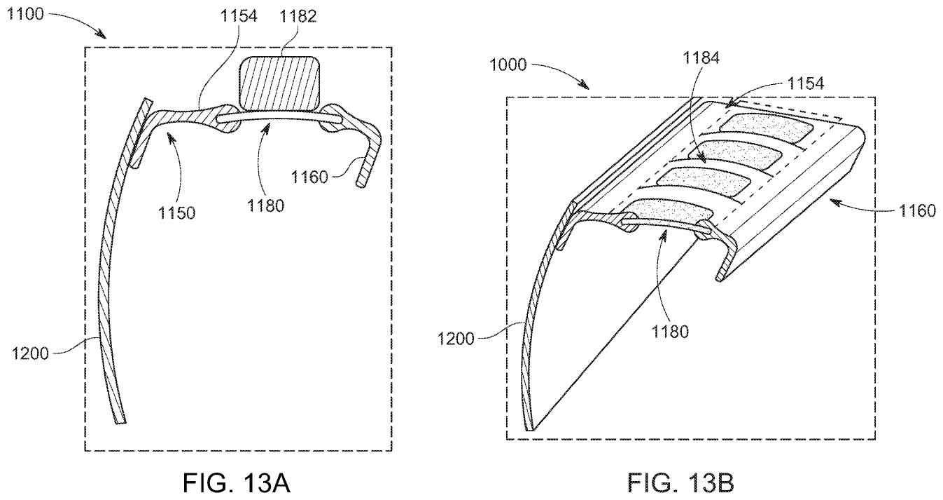

FIG. 13A and FIG. 13B show exemplary venting arrangements according to an aspect of the present technology.

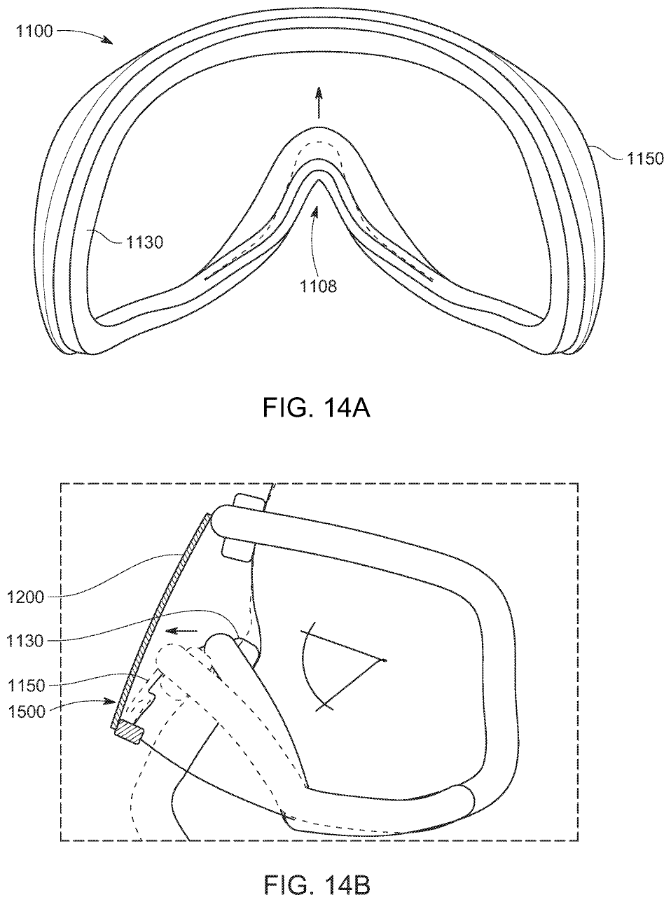

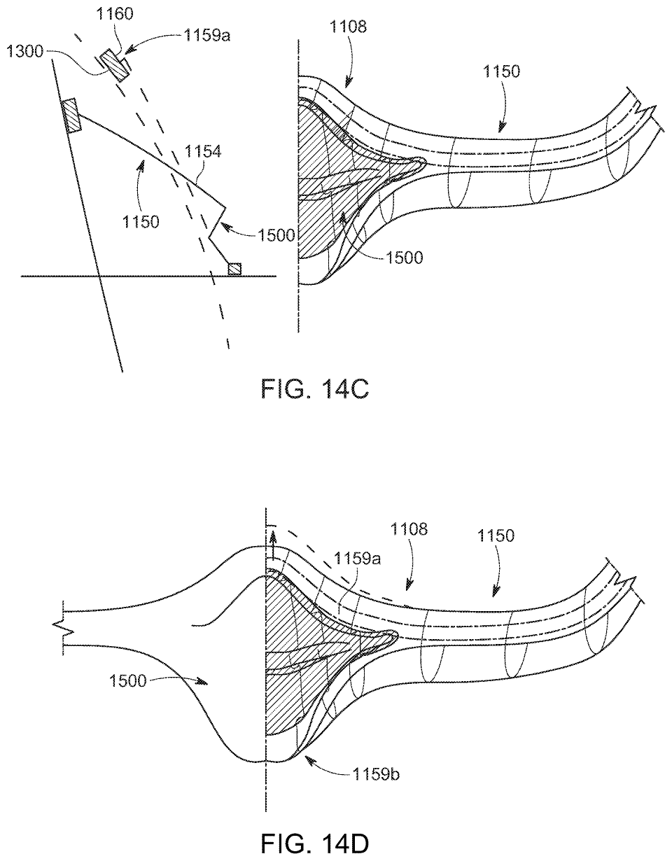

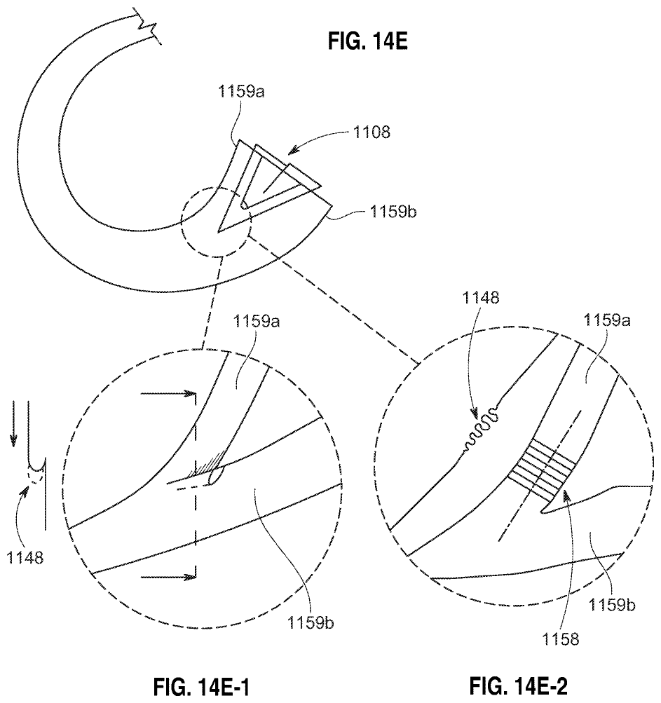

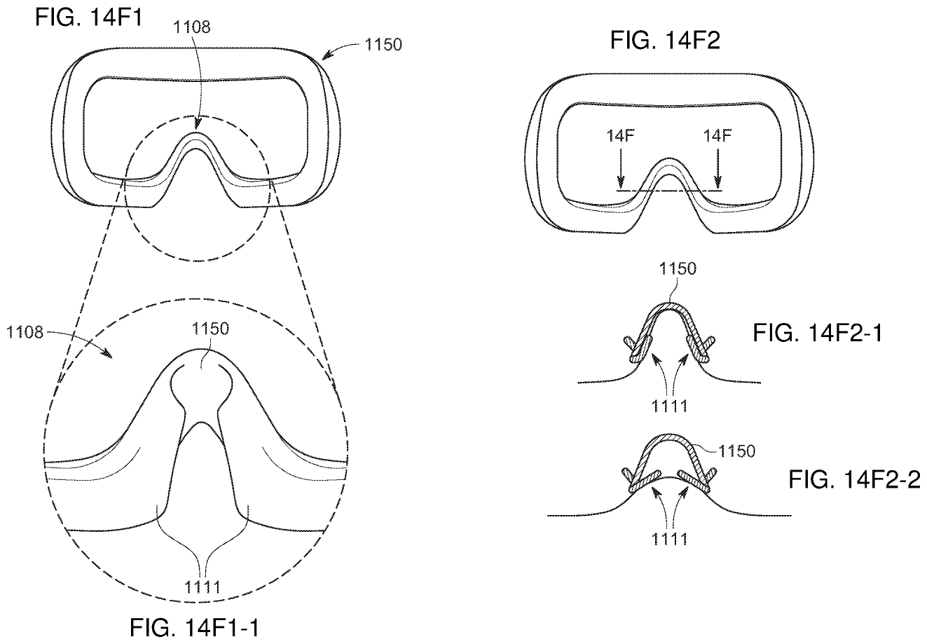

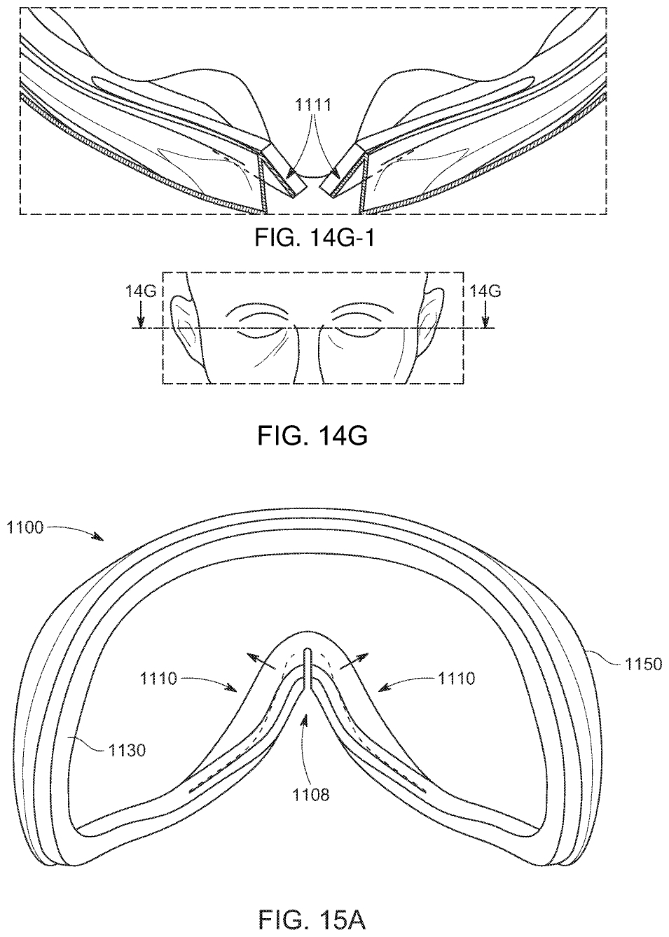

FIG. 14A to FIG. 14G-1 show exemplary interfacing structures according to aspects of the present technology.

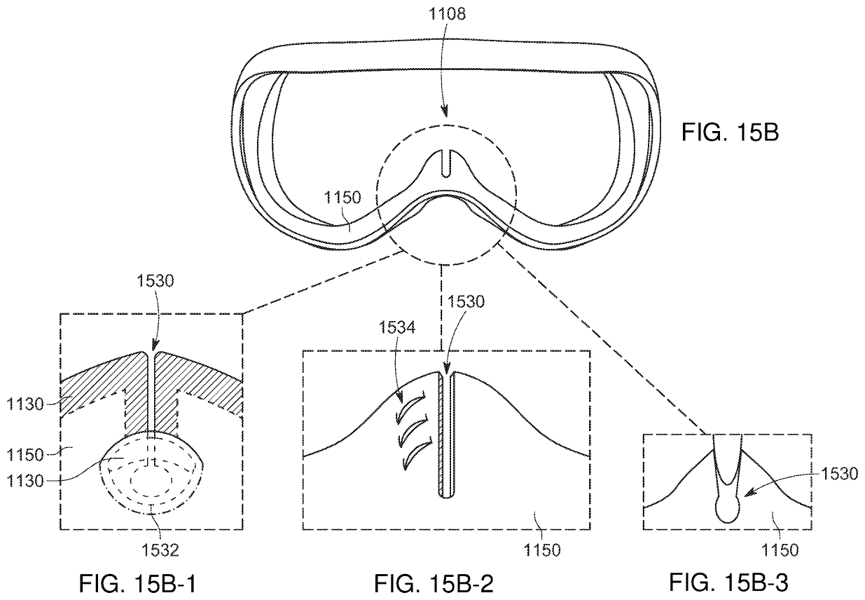

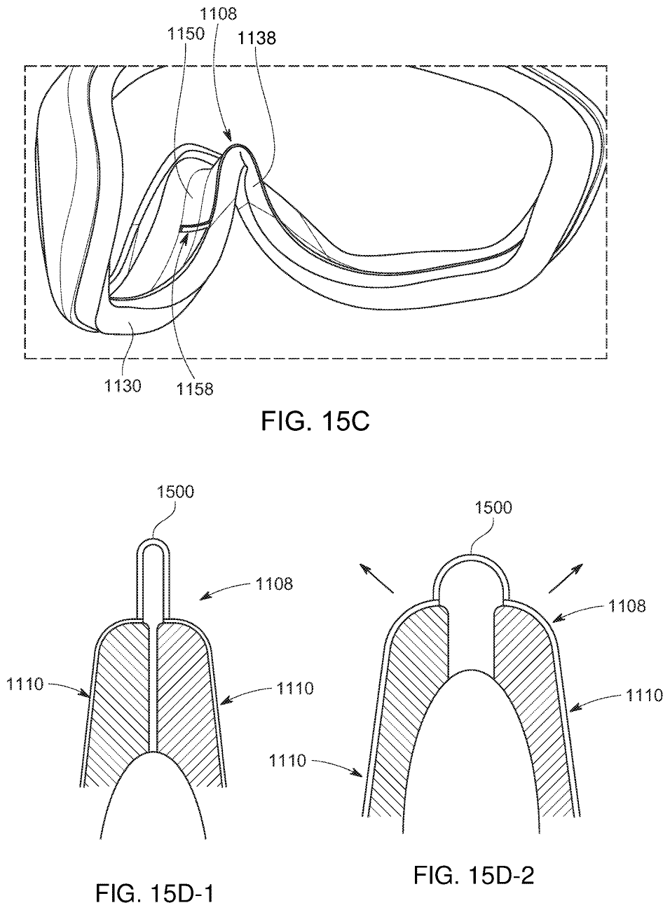

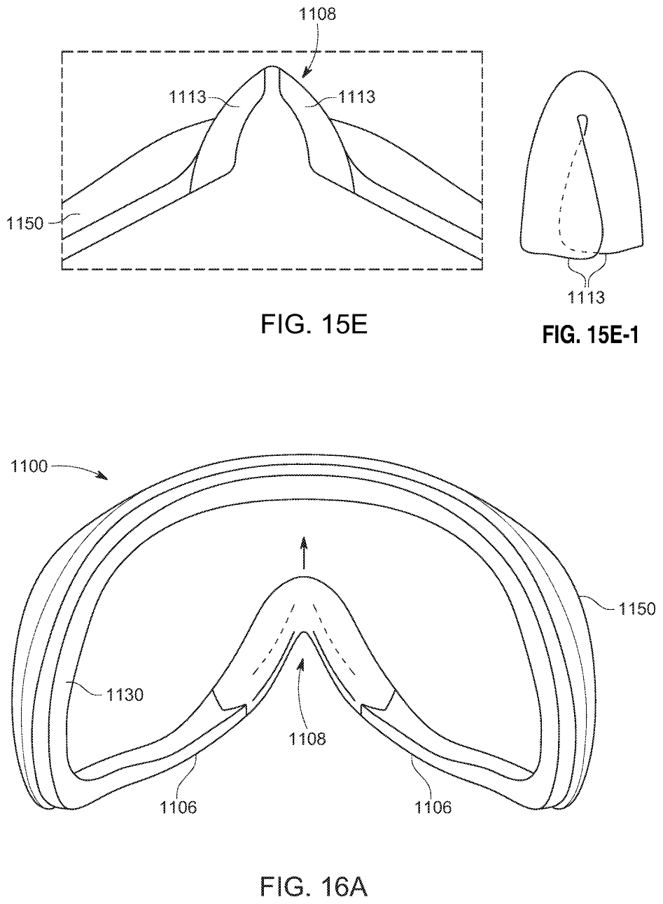

FIG. 15A to FIG. 15E-1 show exemplary interfacing structures according to aspects of the present technology.

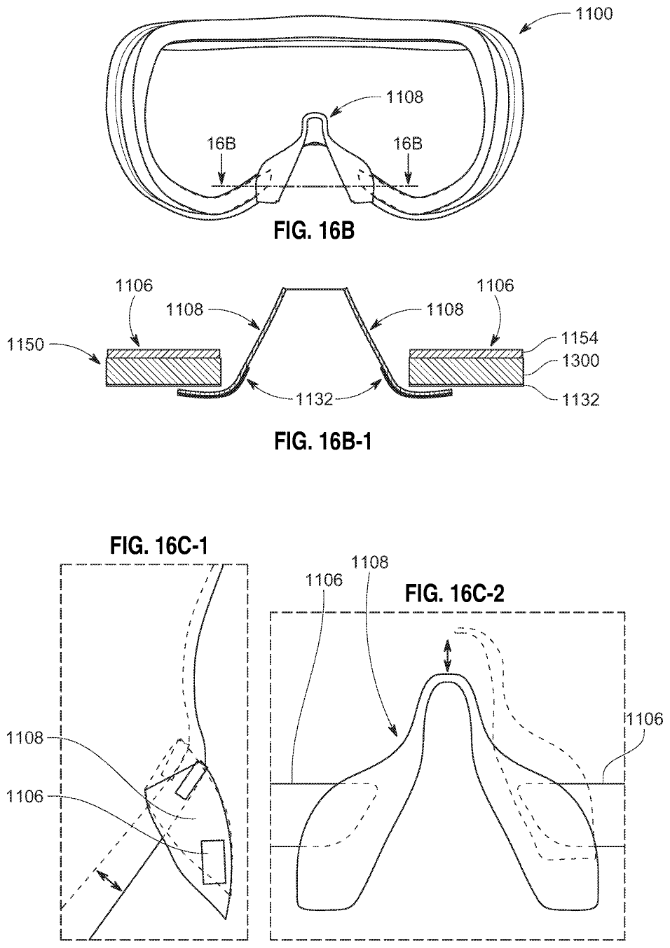

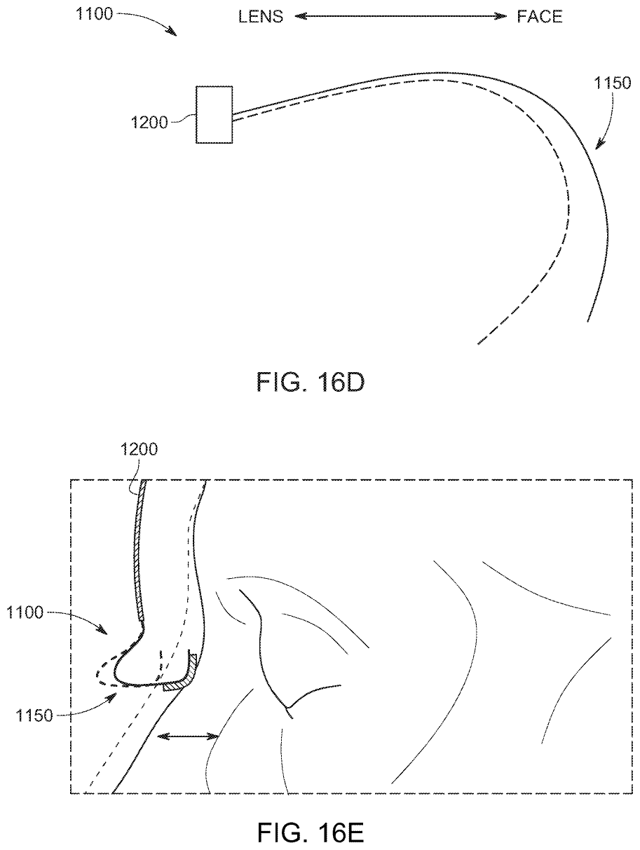

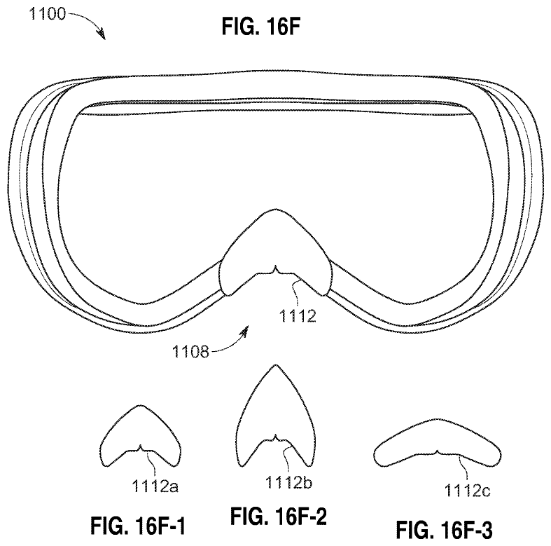

FIG. 16A to FIG. 16F-3 show exemplary interfacing structures according to aspects of the present technology.

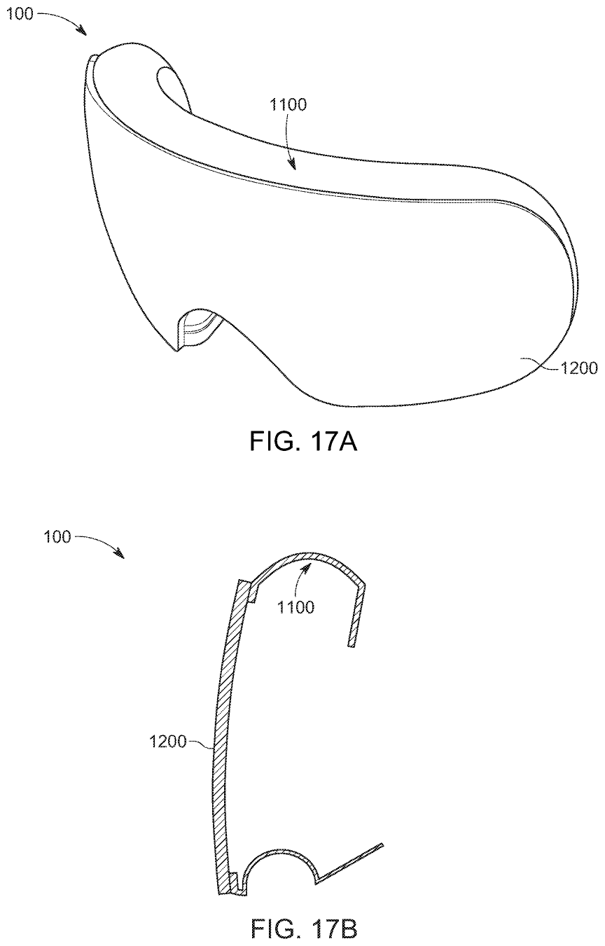

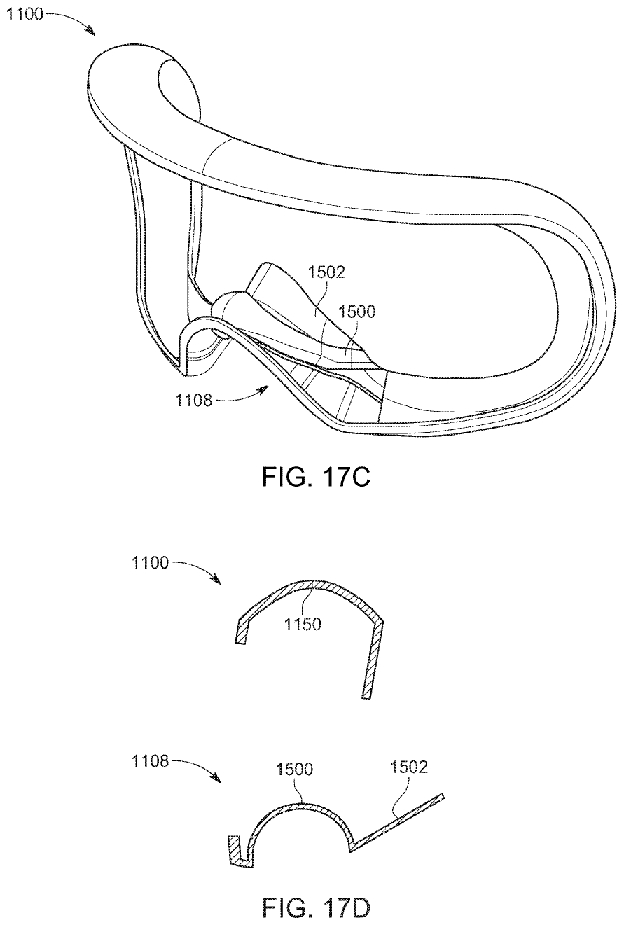

FIG. 17A to FIG. 17D show exemplary interfacing structures according to aspects of the present technology.

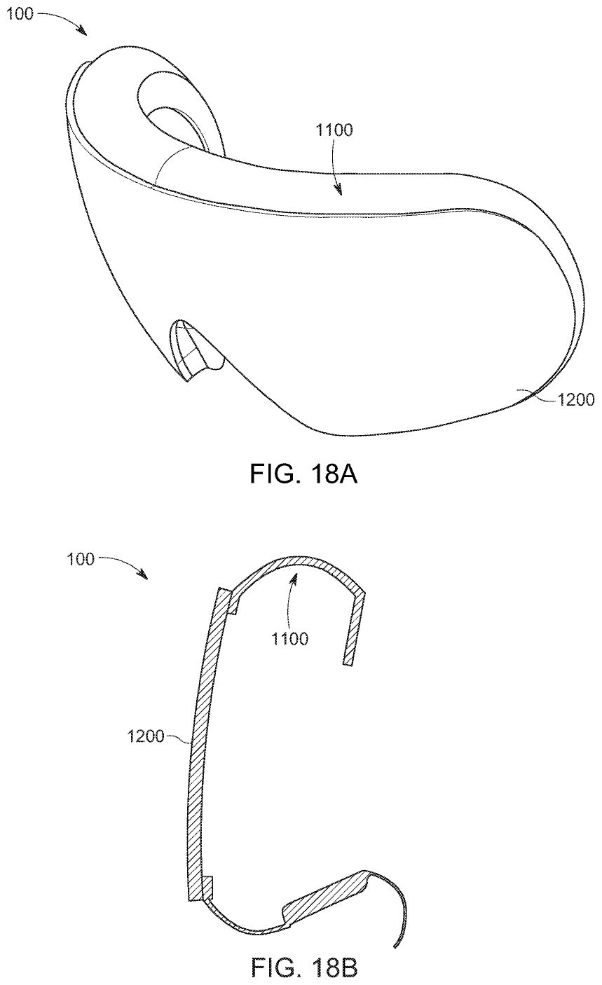

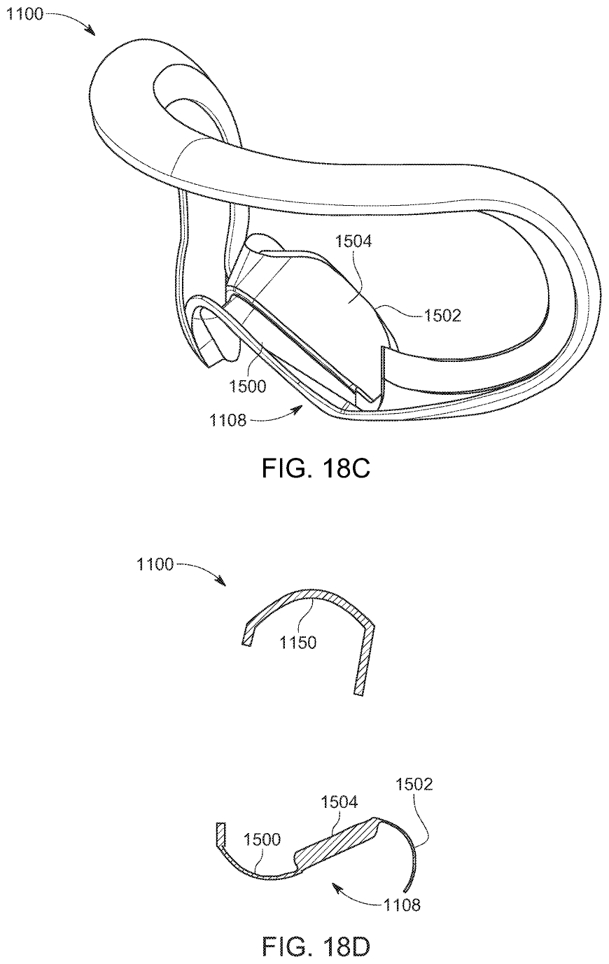

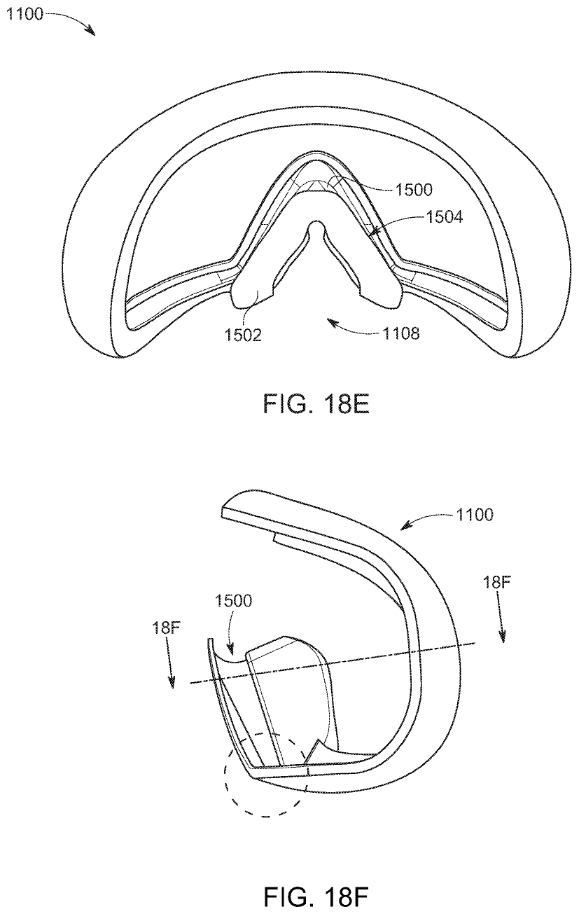

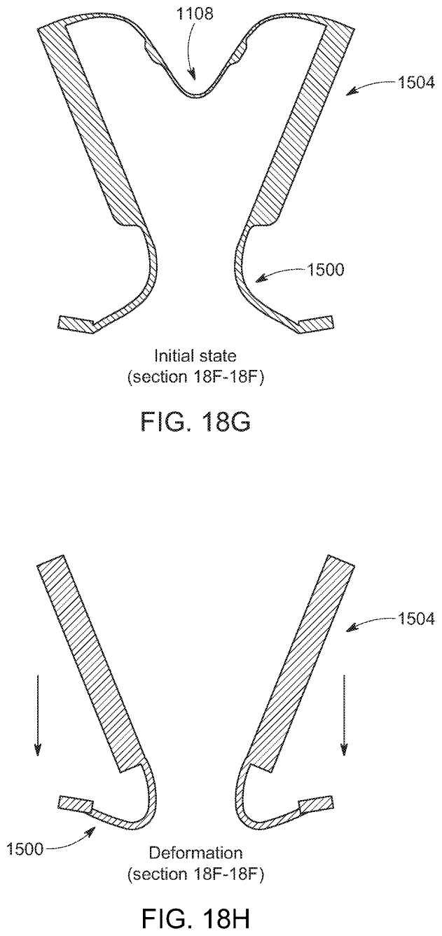

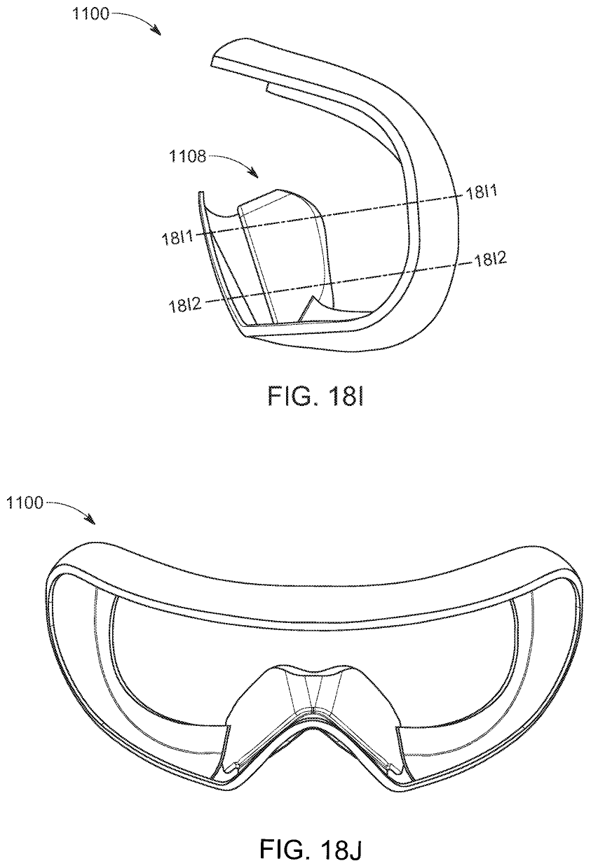

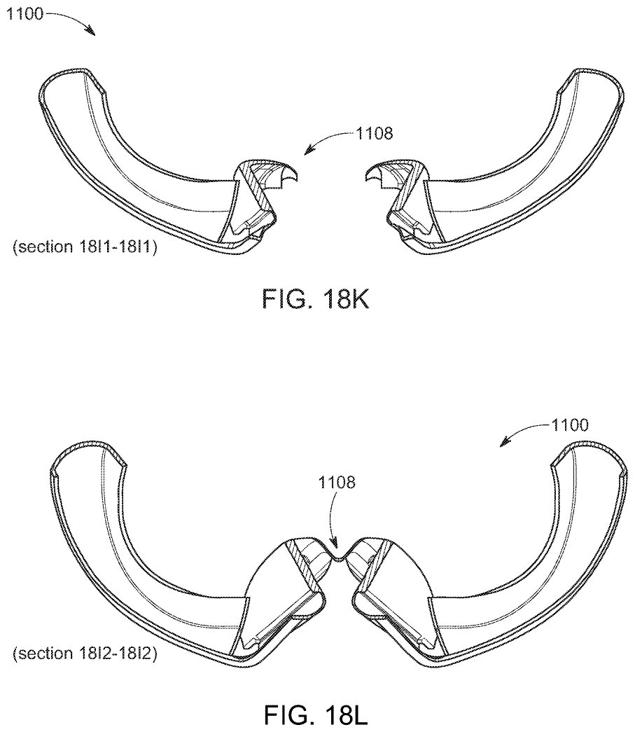

FIG. 18A to FIG. 18L show exemplary interfacing structures according to aspects of the present technology.

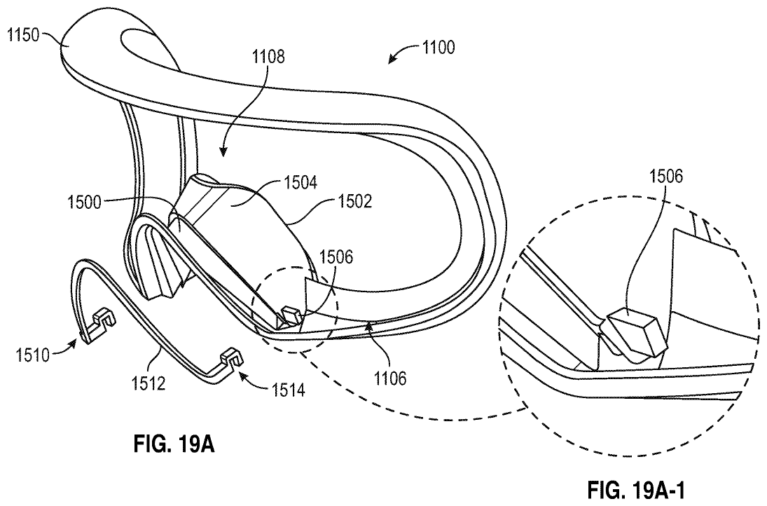

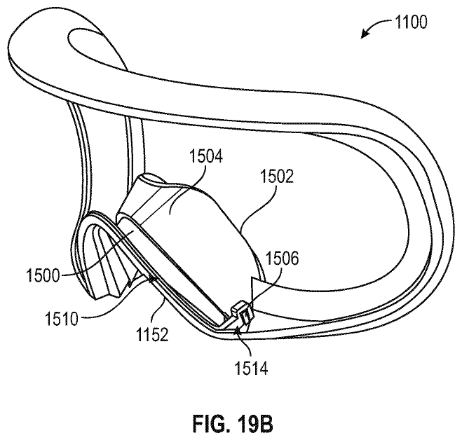

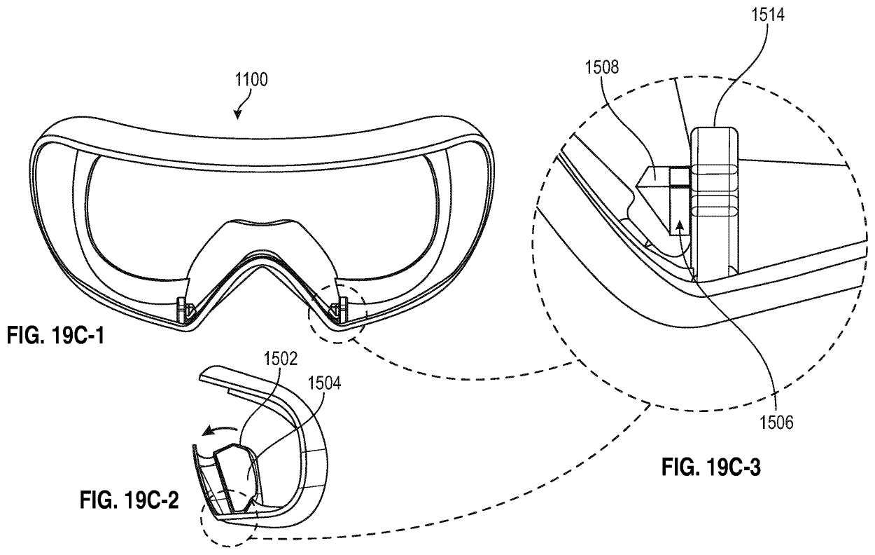

FIG. 19A to FIG. 19C-3 show exemplary interfacing structures according to aspects of the present technology.

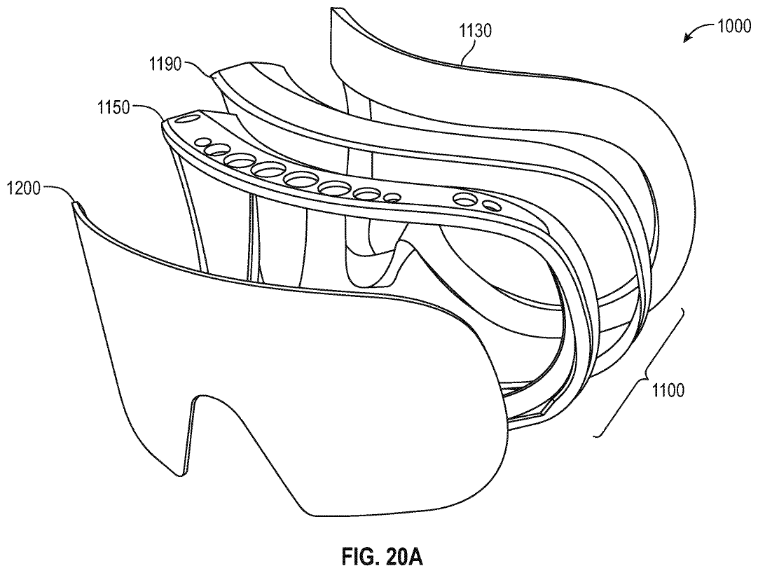

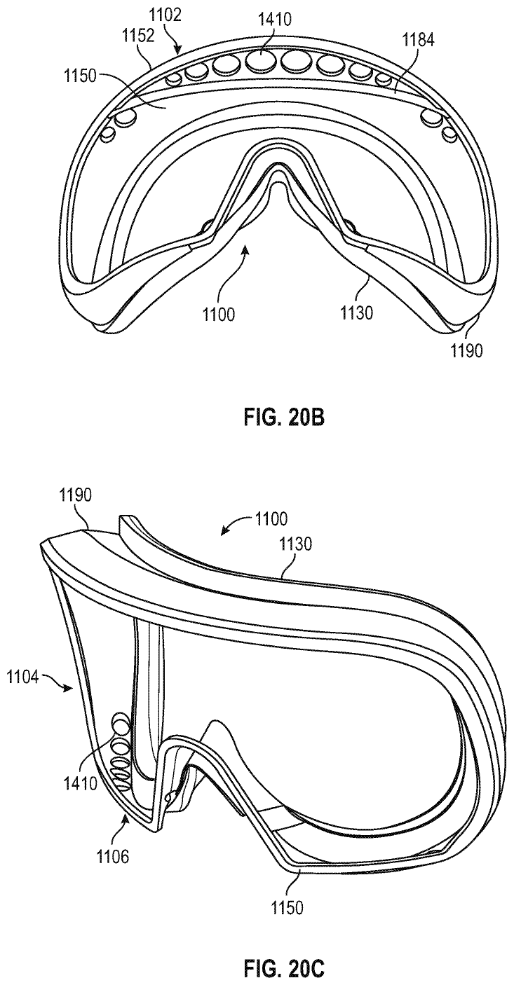

FIG. 20A to FIG. 20E show exemplary interfacing structures according to aspects of the present technology.

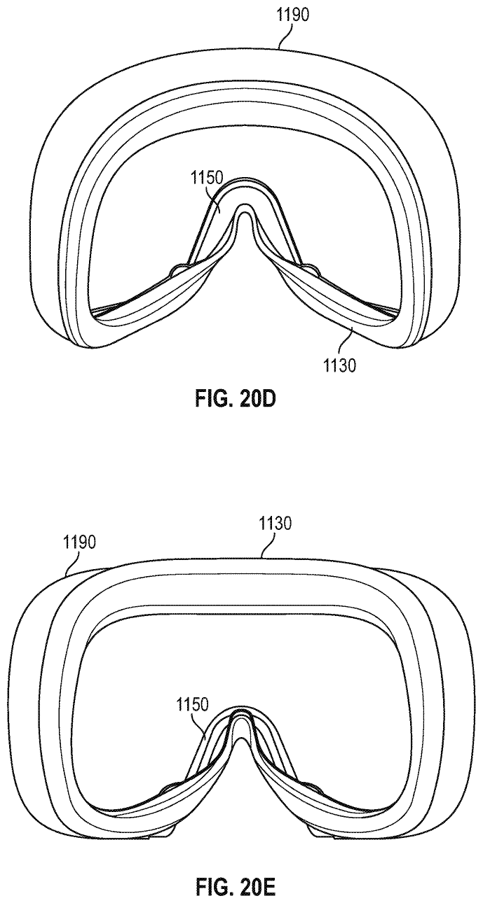

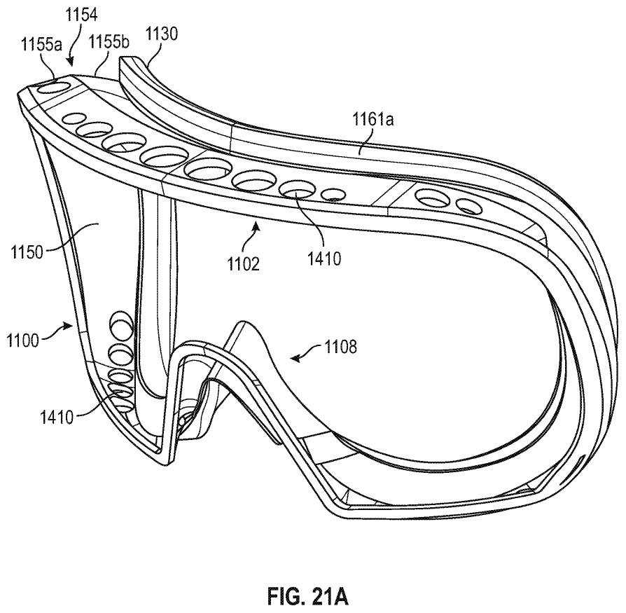

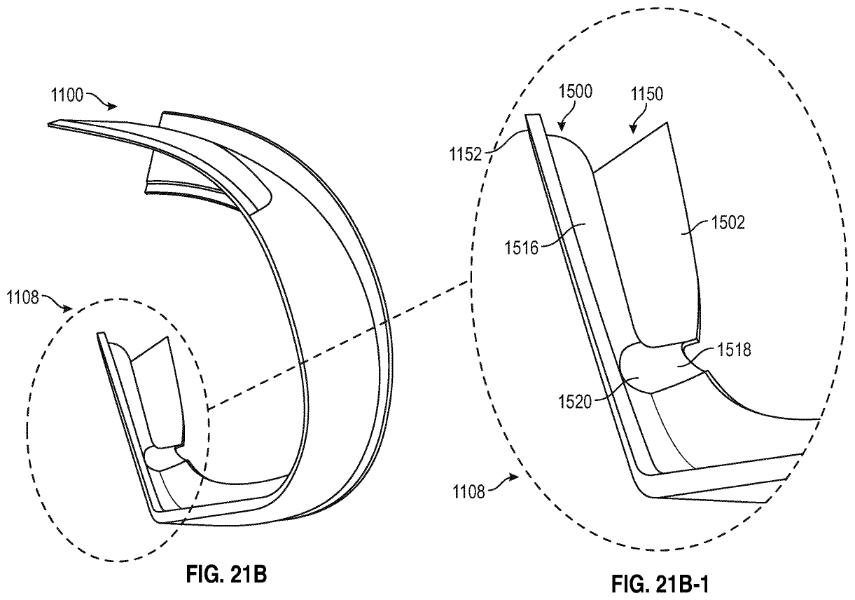

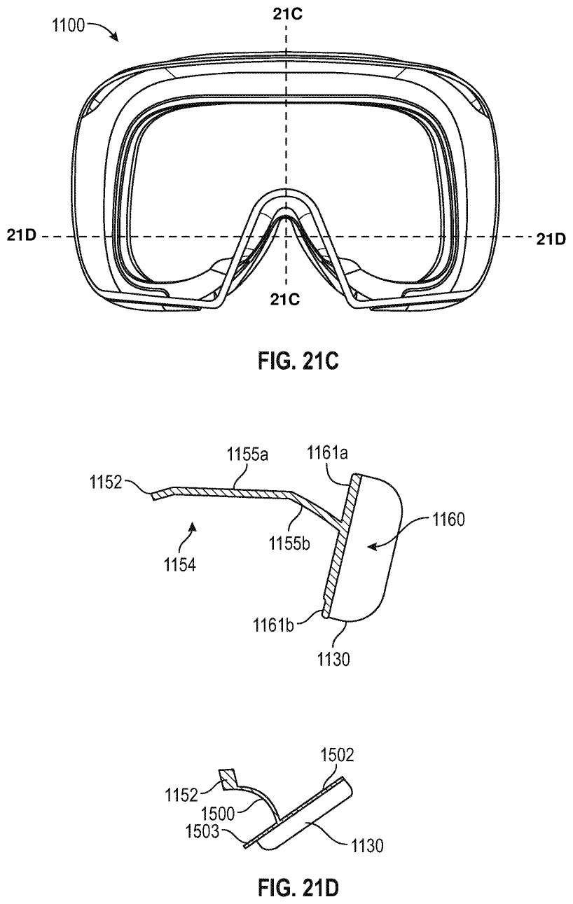

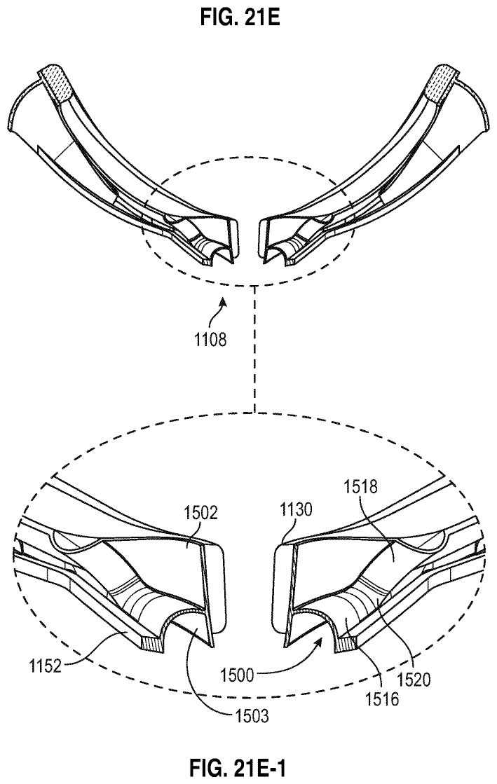

FIG. 21A to FIG. 21E-1 show exemplary interfacing structures according to aspects of the present technology.

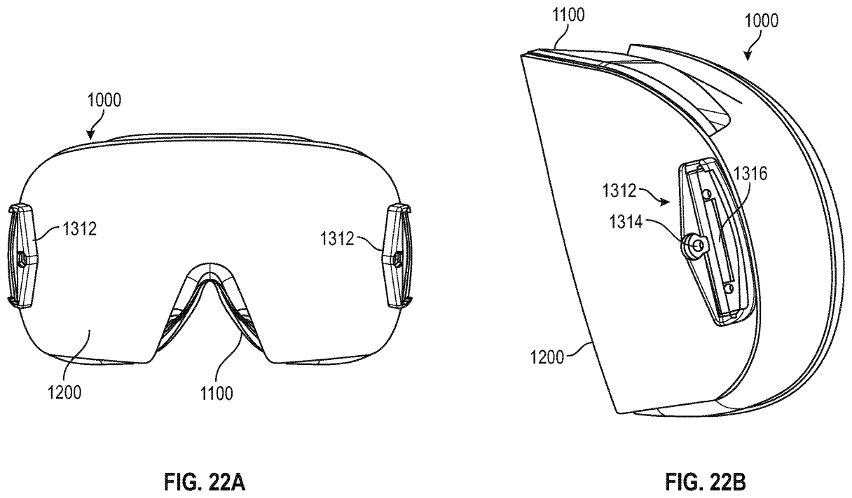

FIG. 22A and FIG. 22B show exemplary eyewear in the form of goggles comprising an exemplary interfacing structure according to aspects of the present technology.

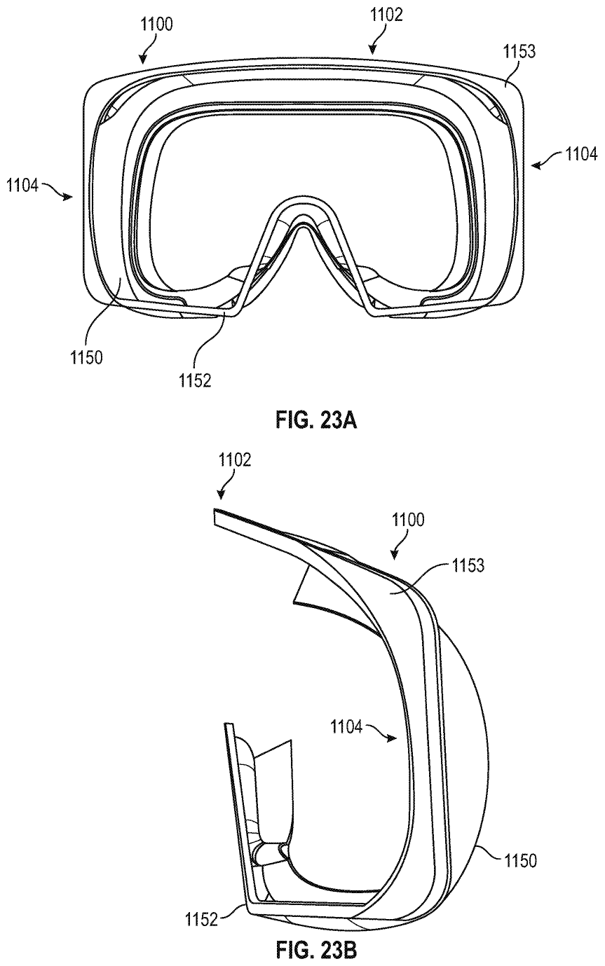

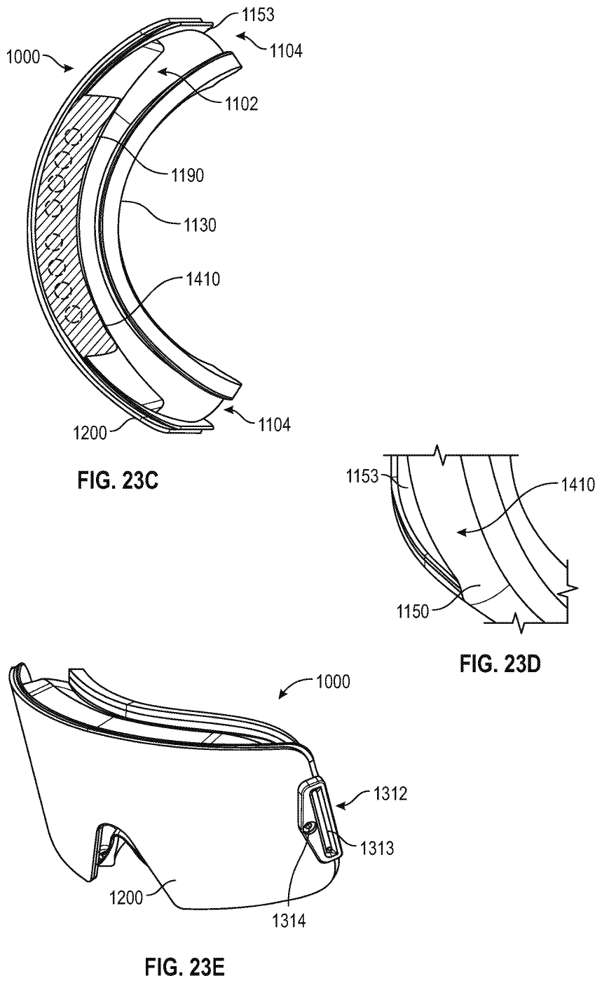

FIG. 23A to FIG. 23D show exemplary interfacing structures according to aspects of the present technology.

FIG. 23E shows exemplary eyewear in the form of goggles comprising the interfacing structure of FIGS. 23A to 23D.

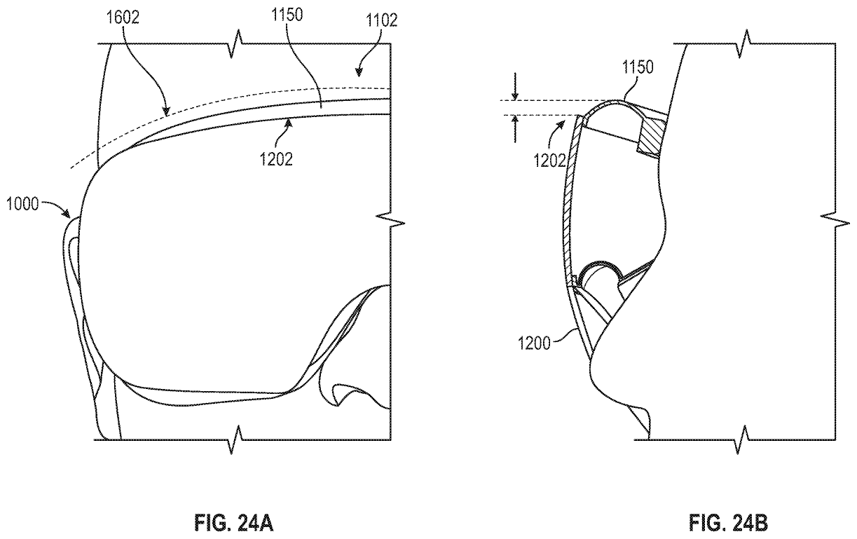

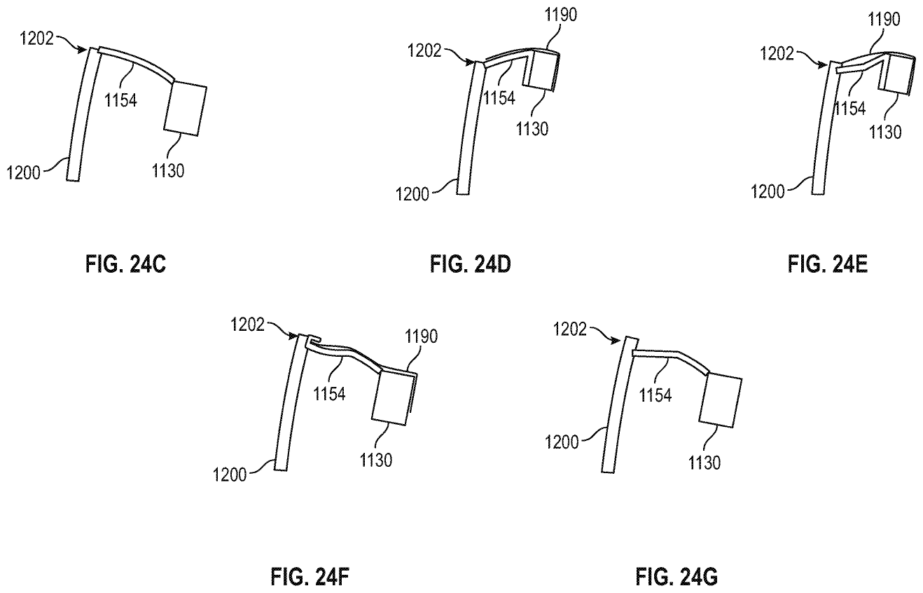

FIG. 24A and FIG. 24B shows exemplary eyewear in the form of goggles comprising an exemplary interfacing structure according to aspects of the present technology.

FIG. 24C to FIG. 24G show exemplary interfacing structures according to aspects of the present technology.

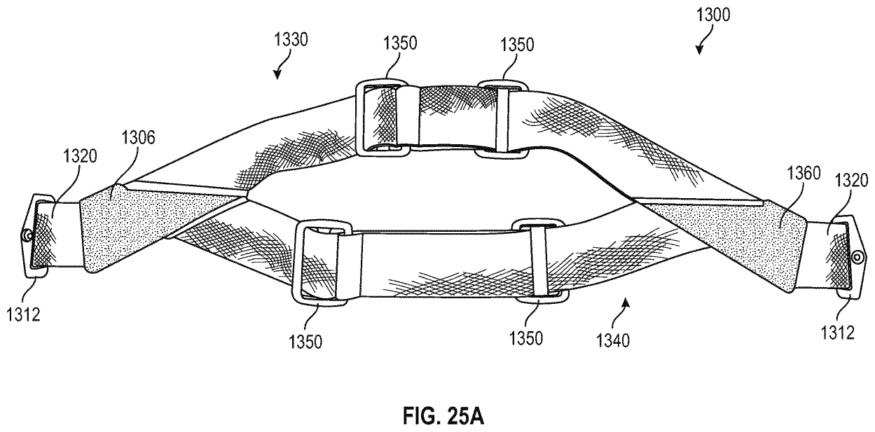

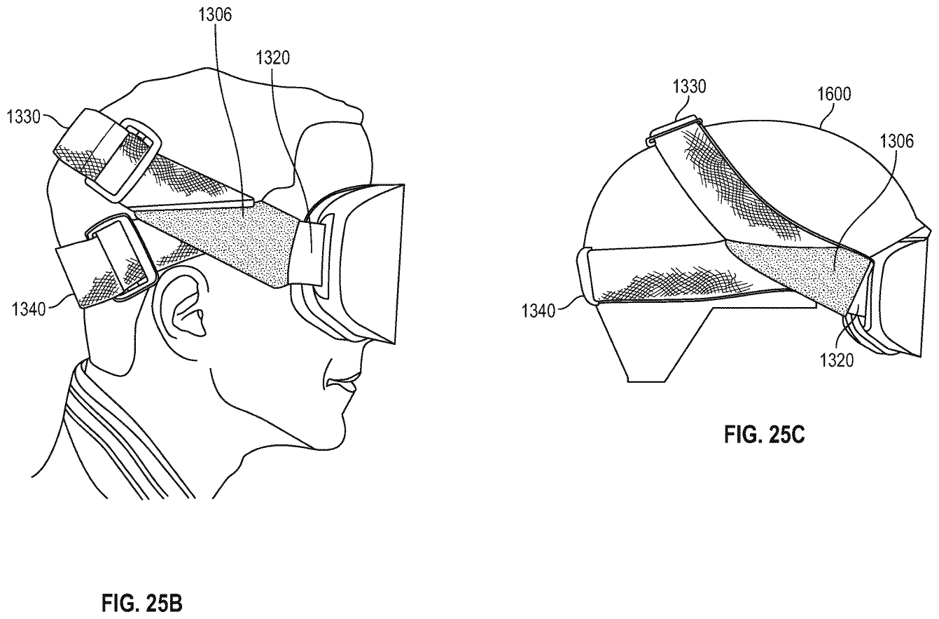

FIG. 25A shows an exemplary positioning and stabilising structure according to an aspect of the present technology.

FIG. 25B shows exemplary eyewear in the form of goggles comprising the positioning and stabilising structure of FIG. 25A when worn by a user.

FIG. 25C shows exemplary eyewear in the form of goggles comprising the positioning and stabilising structure of FIG. 25A when worn with a helmet.

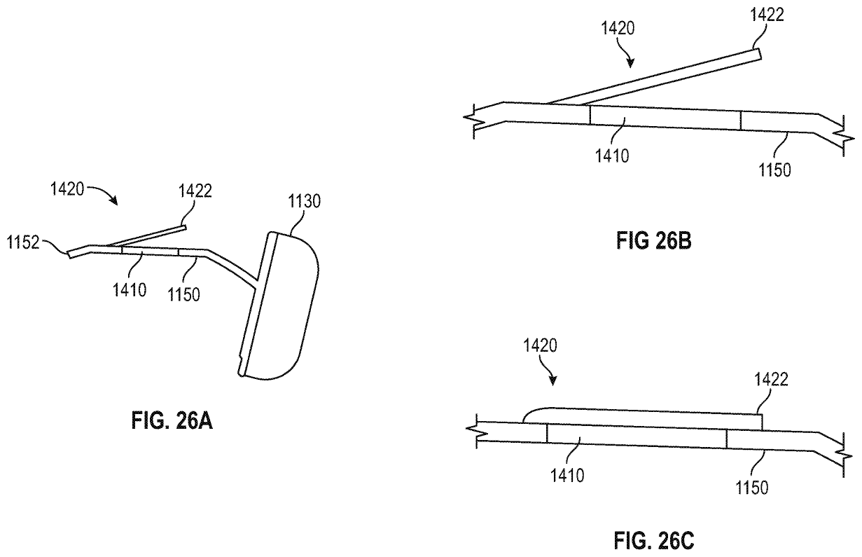

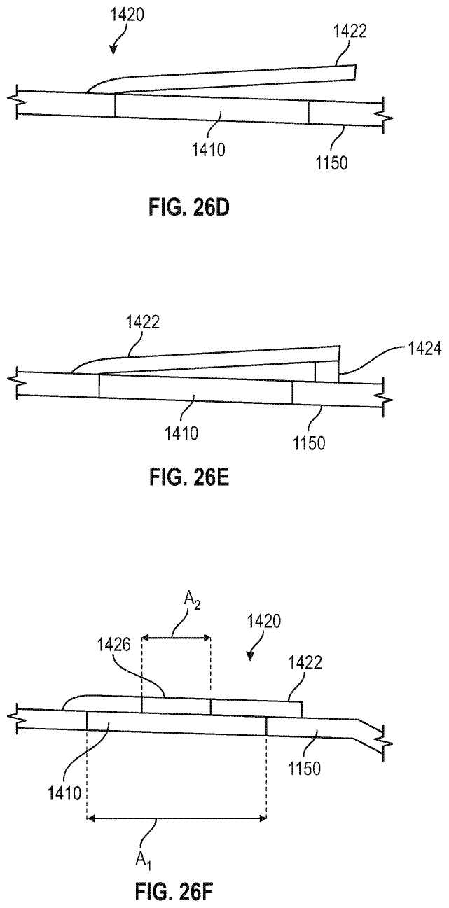

FIG. 26A to FIG. 26E show exemplary interfacing structures according to aspects of the present technology.

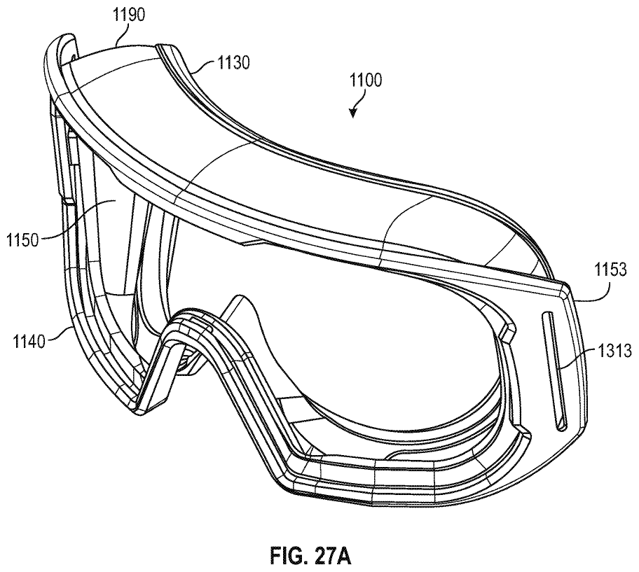

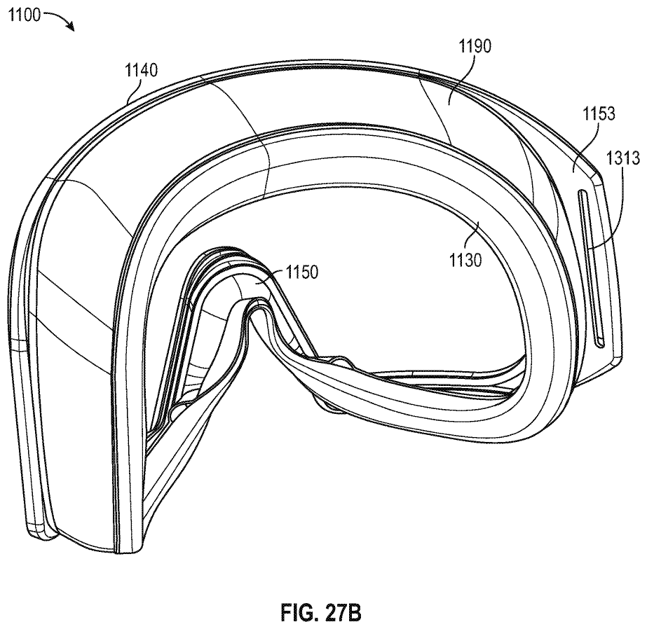

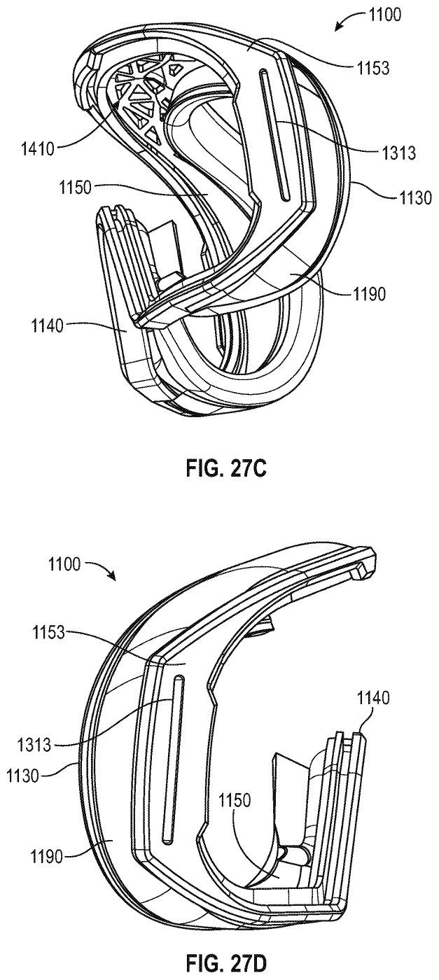

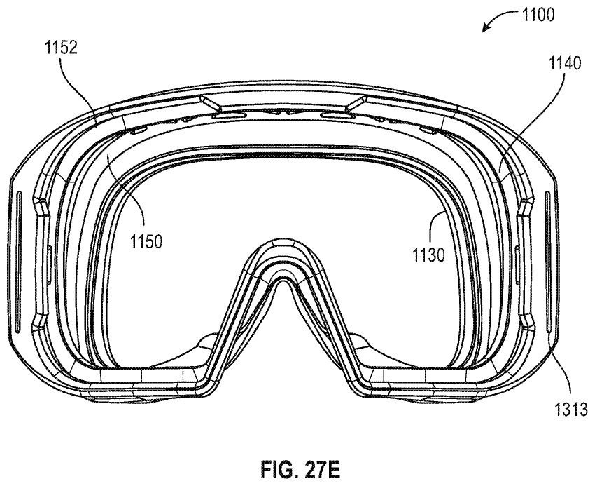

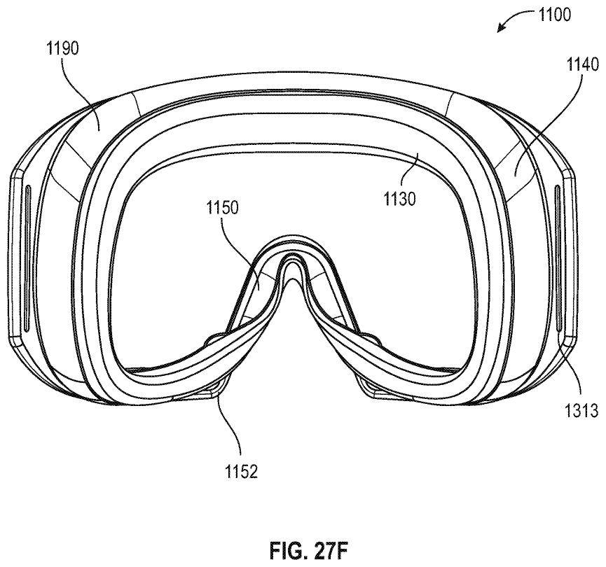

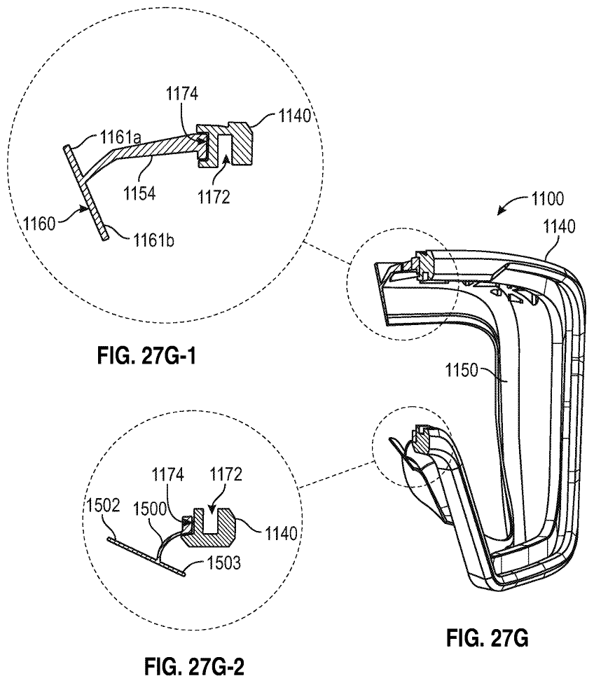

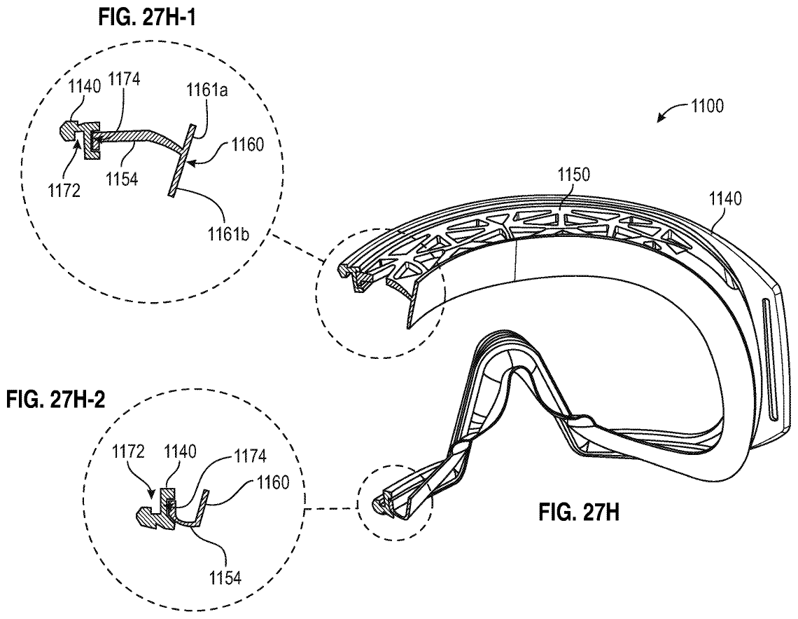

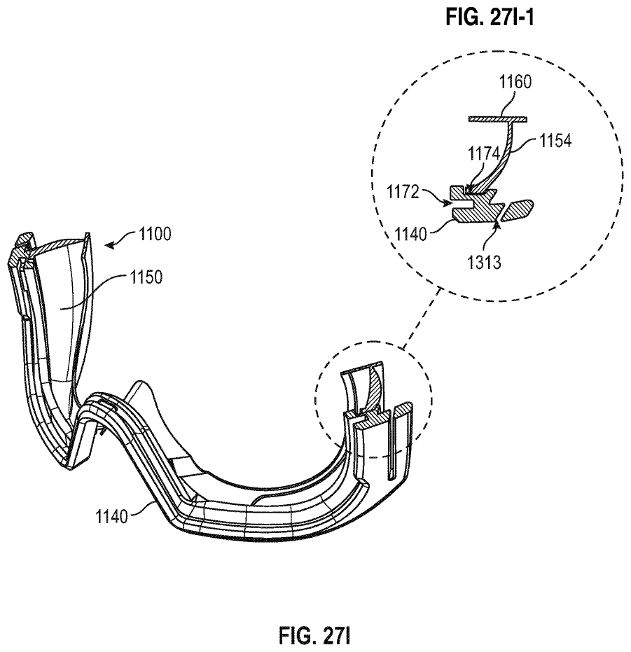

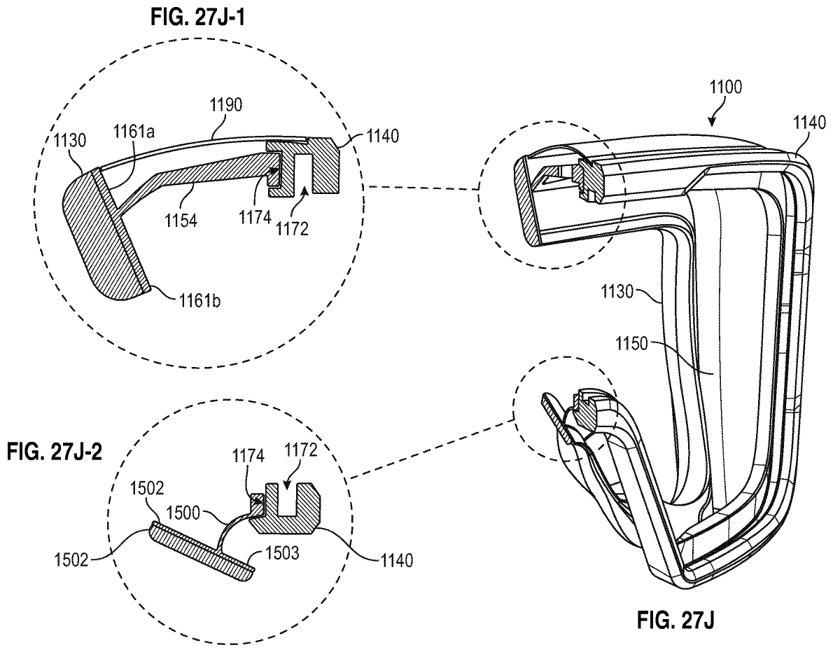

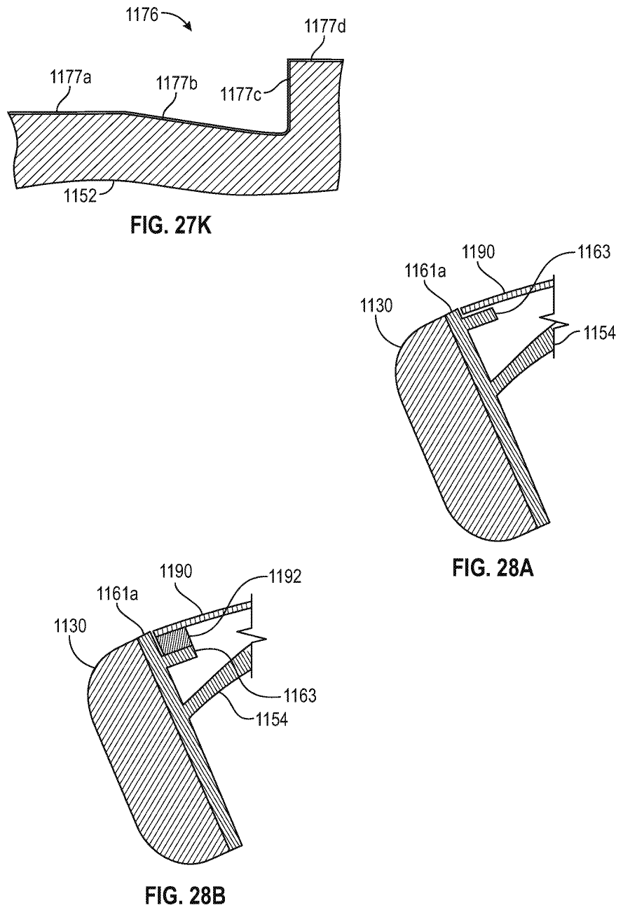

FIG. 27A to FIG. 27K show exemplary interfacing structures according to aspects of the present technology.

FIG. 28A and FIG. 28B are cross-sectional views of exemplary interfacing structures according to aspects of the present technology.

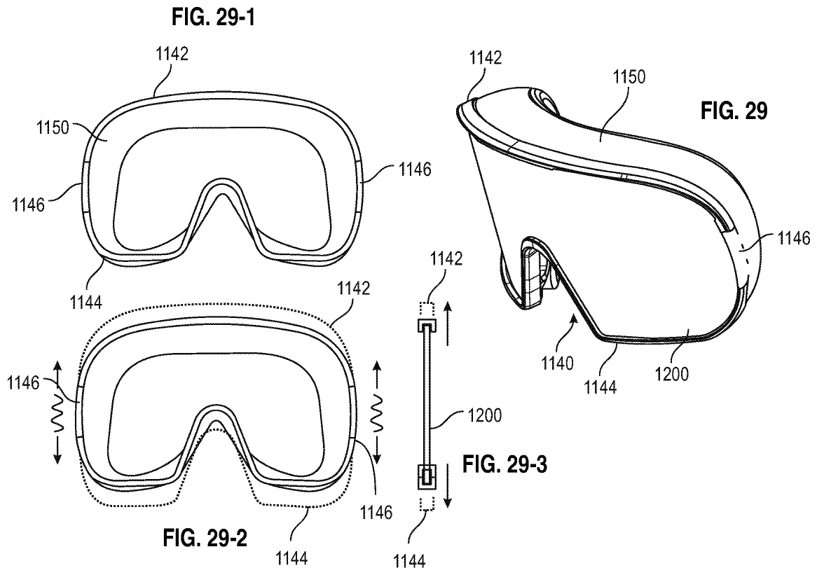

FIG. 29 to FIG. 29-3 show an exemplary interfacing structure according to aspects of the present technology.

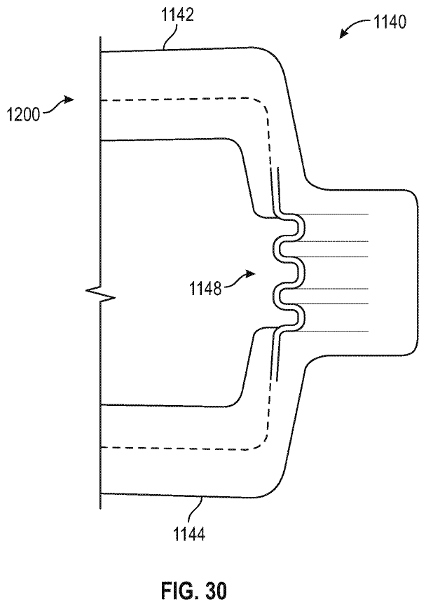

FIG. 30 shows an exemplary chassis portion of an interfacing structure according to aspects of the present technology.

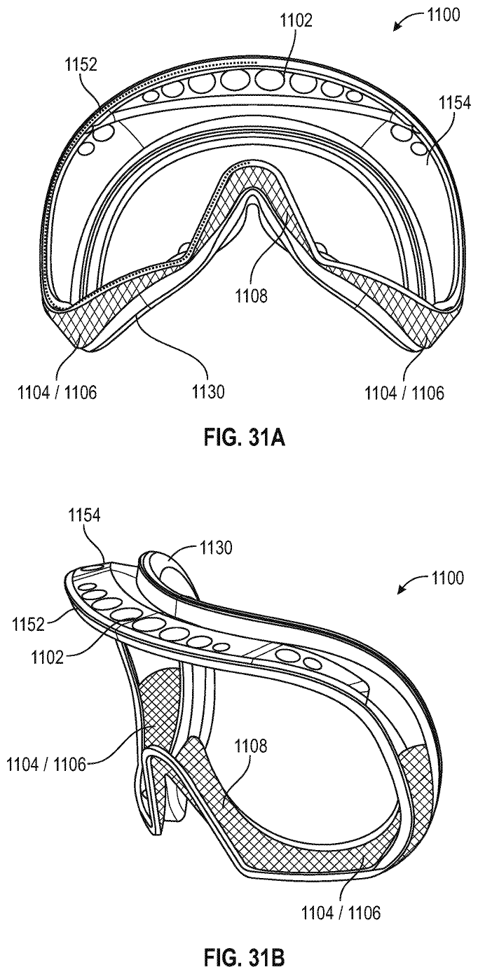

FIG. 31A and FIG. 31B show an exemplary interfacing structure according to aspects of the present technology.

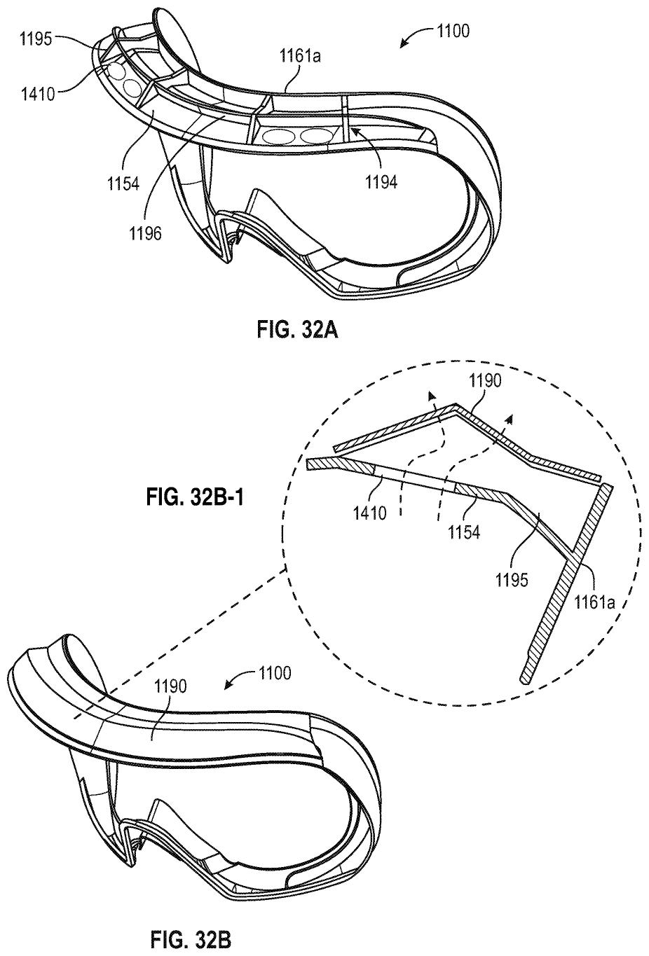

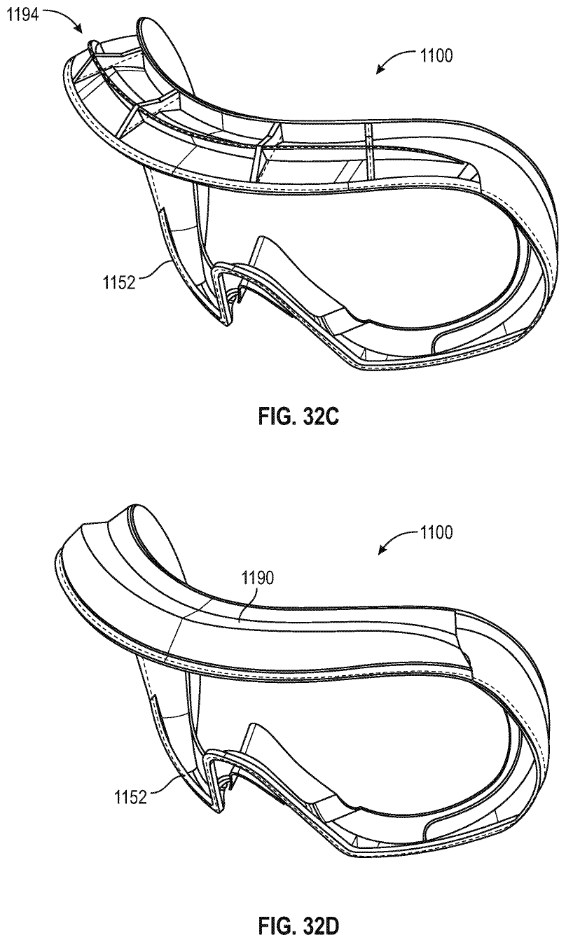

FIG. 32A to FIG. 32D show exemplary interfacing structures according to aspects of the present technology.

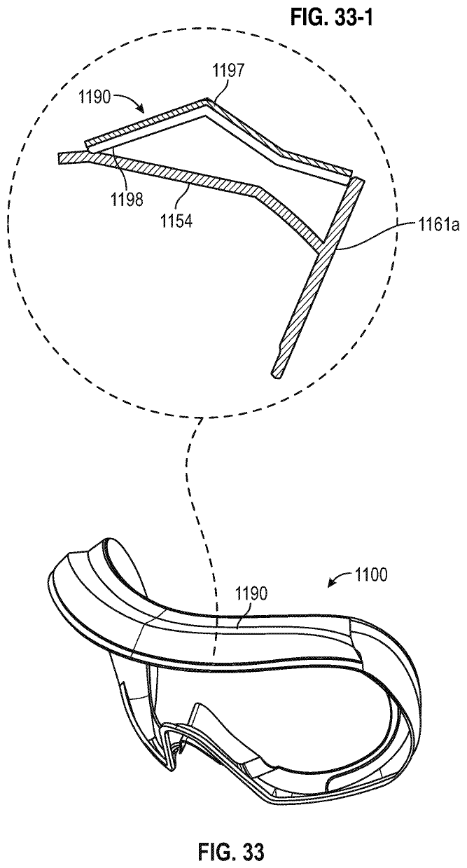

FIG. 33 and FIG. 33-1 show an exemplary interfacing structure according to aspects of the present technology.

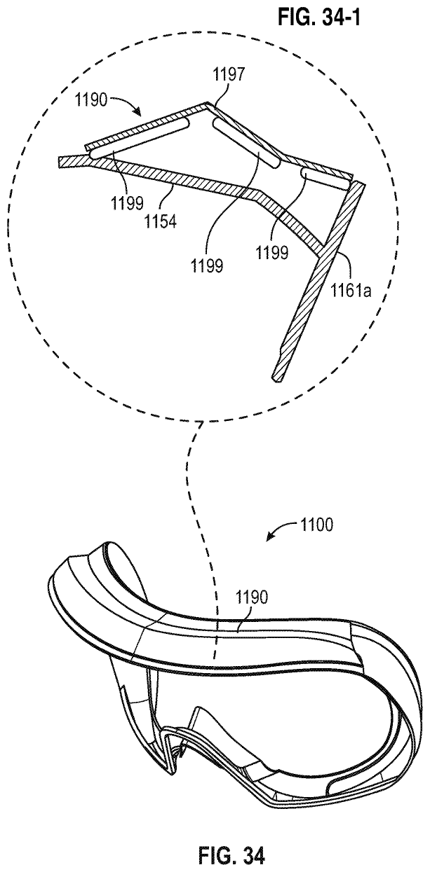

FIG. 34 and FIG. 34-1 show an exemplary interfacing structure according to aspects of the present technology.

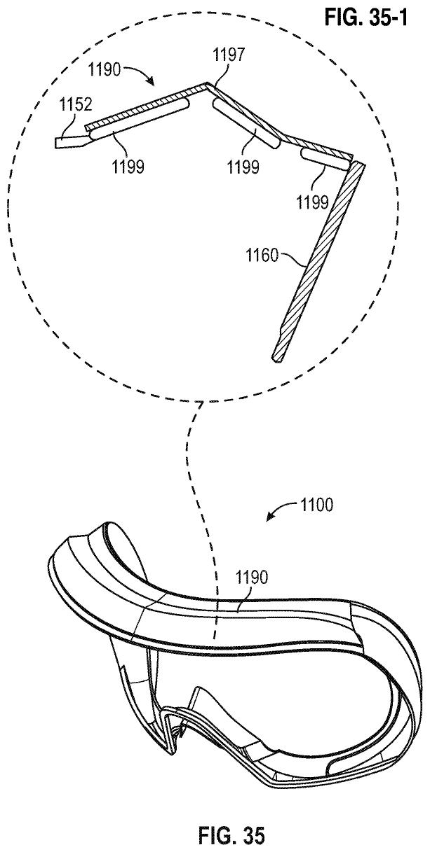

FIG. 35 and FIG. 35-1 show an exemplary interfacing structure according to aspects of the present technology.

### 4.2 Facial Anatomy

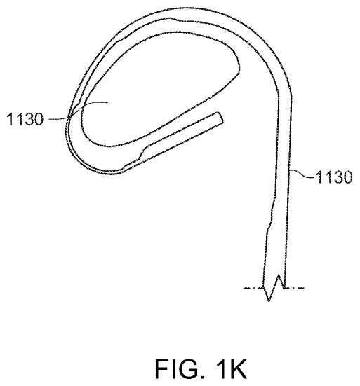

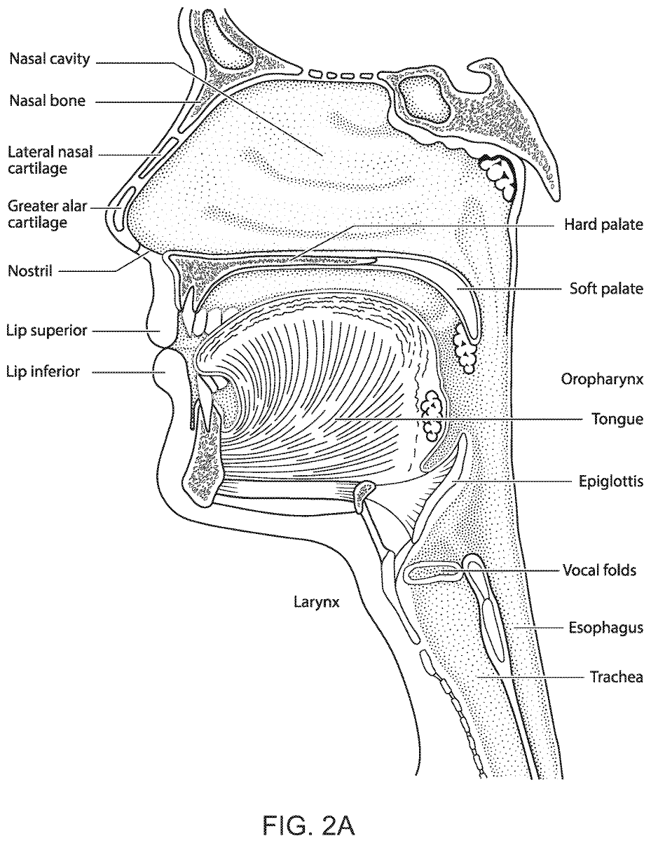

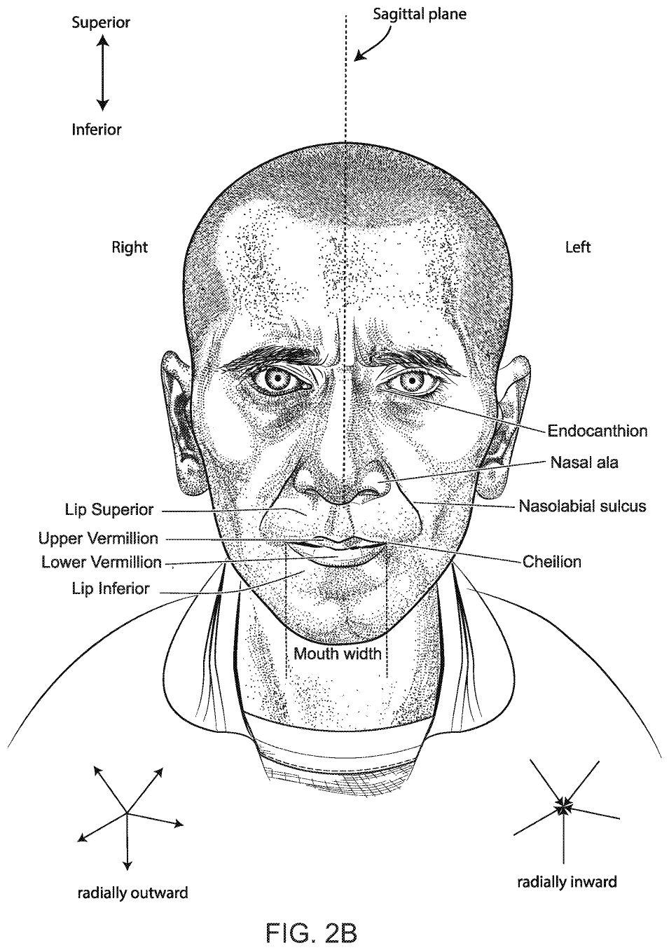

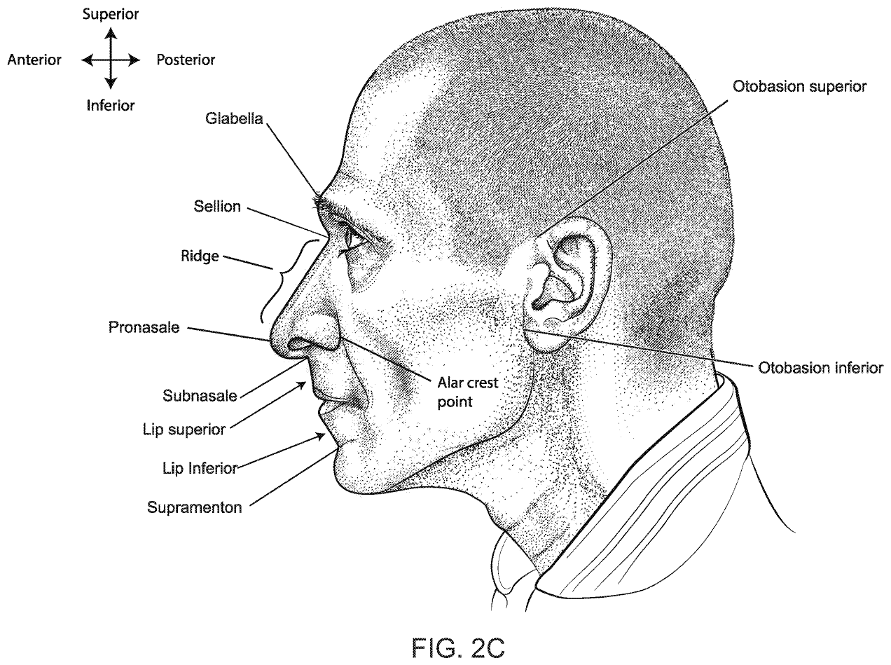

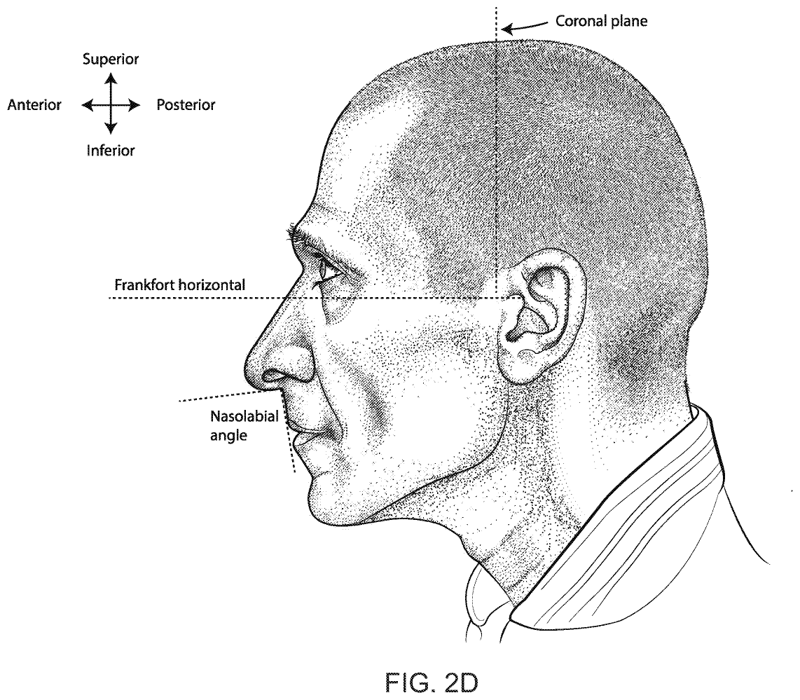

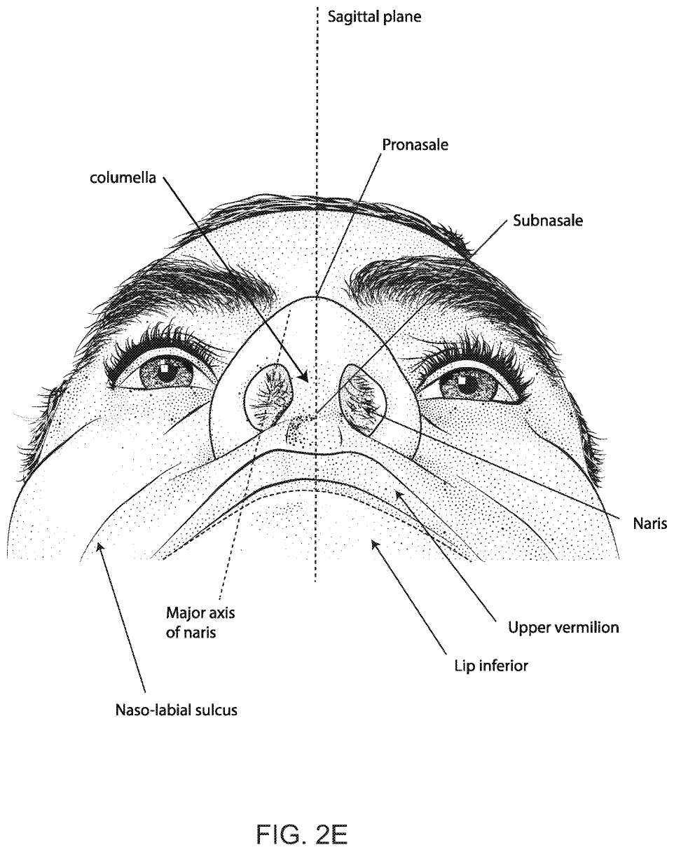

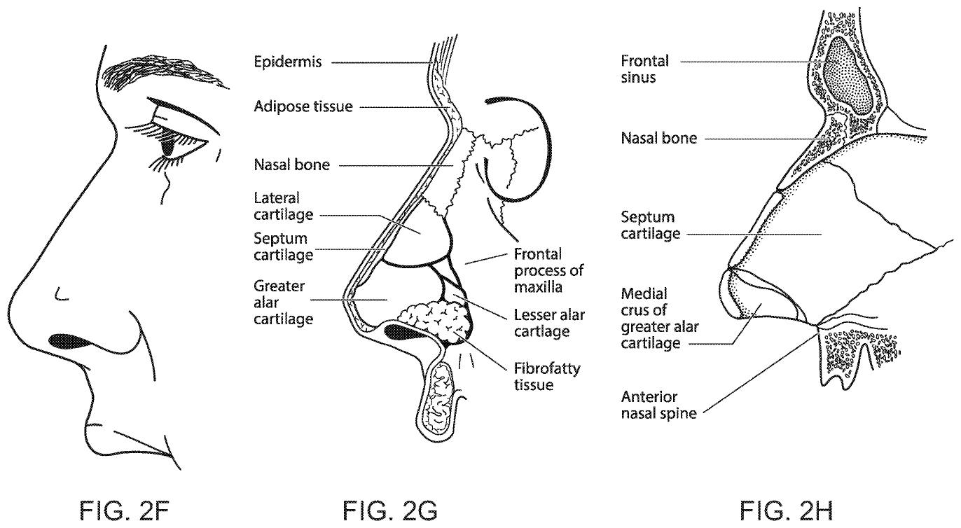

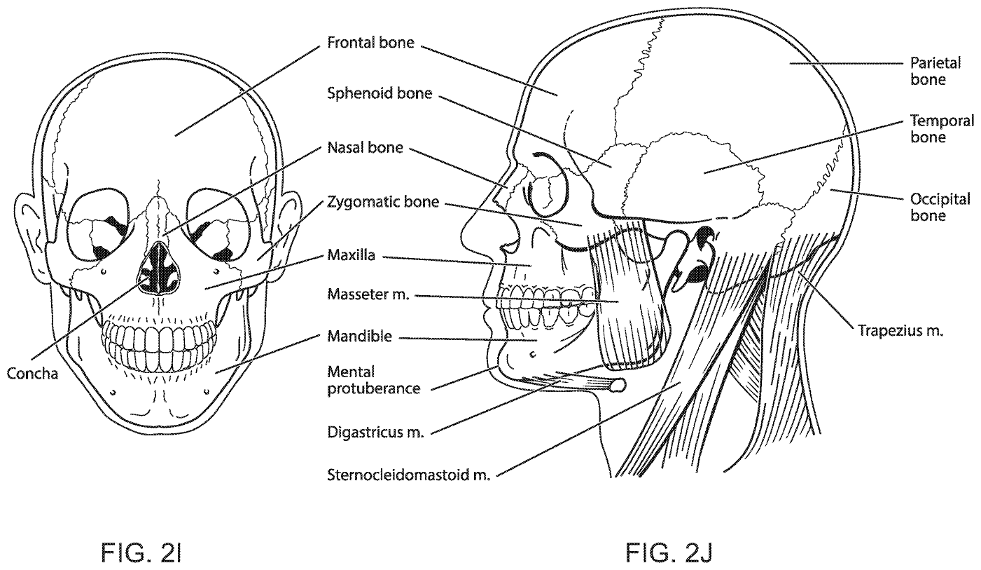

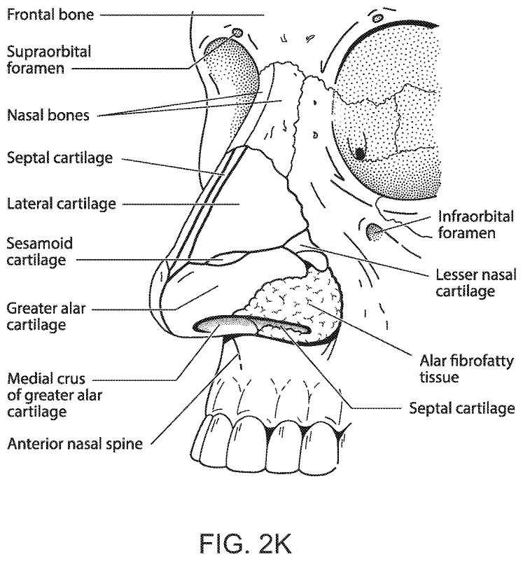

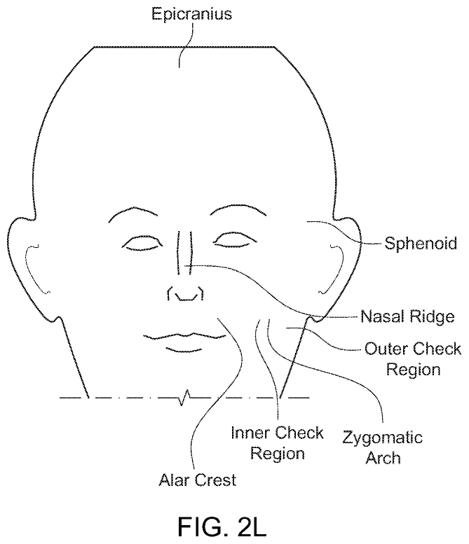

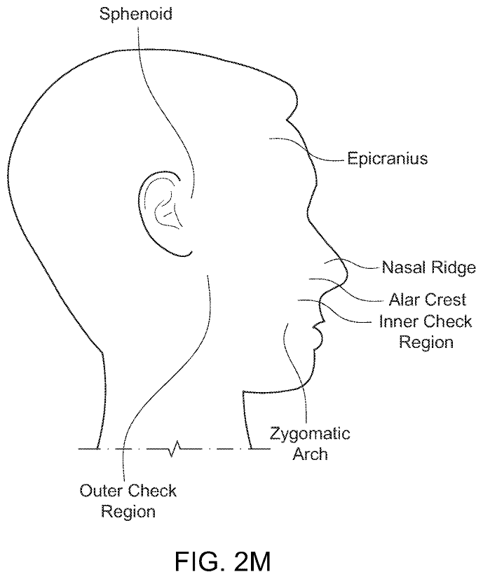

FIG. 2A shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2B is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2C is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2D is a further side view of a head. The approximate locations of the Frankfort horizontal and nasolabial angle are indicated. The coronal plane is also indicated.

FIG. 2E shows a base view of a nose with several features identified including naso-labial sulcus, lip inferior, upper Vermilion, naris, subnasale, columella, pronasale, the major axis of a naris and the midsagittal plane.

FIG. 2F shows a side view of the superficial features of a nose.

FIG. 2G shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2H shows a medial dissection of a nose, approximately several millimeters from the midsagittal plane, amongst other things showing the septum cartilage and medial crus of greater alar cartilage.

FIG. 2I shows a front view of the bones of a skull including the frontal, nasal and zygomatic bones. Nasal concha are indicated, as are the maxilla, and mandible.

FIG. 2J shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2K shows an anterolateral view of a nose. The following bones are shown: frontal, supraorbital foramen, nasal, septal cartilage, lateral cartilage, orbit and infraorbital foramen.

FIG. 2L shows another front view of the face with several features of surface anatomy identified including the epicranius, the sphenoid, the nasal ridge, the outer and inner cheek regions, the zygomatic arch, and the alar crest.

FIG. 2M shows another side view of the face with several features of surface anatomy identified including the epicranius, the sphenoid, the nasal ridge, the outer and inner cheek regions, the zygomatic arch, and the alar crest.

### 4.3 Shape of Structures

FIG. 3A shows a schematic of a cross-section through a structure at a point. An outward normal at the point is indicated. The curvature at the point has a positive sign, and a relatively large magnitude when compared to the magnitude of the curvature shown in FIG. 3B.

FIG. 3B shows a schematic of a cross-section through a structure at a point. An outward normal at the point is indicated. The curvature at the point has a positive sign, and a relatively small magnitude when compared to the magnitude of the curvature shown in FIG. 3A.

FIG. 3C shows a schematic of a cross-section through a structure at a point. An outward normal at the point is indicated. The curvature at the point has a value of zero.

FIG. 3D shows a schematic of a cross-section through a structure at a point. An outward normal at the point is indicated. The curvature at the point has a negative sign, and a relatively small magnitude when compared to the magnitude of the curvature shown in FIG. 3E.

FIG. 3E shows a schematic of a cross-section through a structure at a point. An outward normal at the point is indicated. The curvature at the point has a negative sign, and a relatively large magnitude when compared to the magnitude of the curvature shown in FIG. 3D.

FIG. 3F shows the surface of a structure, with a one dimensional hole in the surface. The illustrated plane curve forms the boundary of a one dimensional hole.

FIG. 3G shows a cross-section through the structure of FIG. 3F. The illustrated surface bounds a two dimensional hole in the structure of FIG. 3F.

FIG. 3H shows a perspective view of the structure of FIG. 3F, including the two dimensional hole and the one dimensional hole. Also shown is the surface that bounds a two dimensional hole in the structure of FIG. 3F.

FIGS. 3I-3J shows a seal forming structure. An exterior surface of the cushion is indicated. An edge of the surface is indicated. A path on the surface between points A and B is indicated. A straight-line distance between A and B is indicated. Two saddle regions and a dome region are indicated.

FIG. 3K illustrates a left-hand rule.

FIG. 3L illustrates a right-hand rule.

FIG. 3M shows a left ear, including the left ear helix.

FIG. 3N shows a right ear, including the right ear helix.

FIG. 3O shows a right-hand helix.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Eyewear

As shown in FIG. 1A, eyewear in the form of goggles 1000 in accordance with one aspect of the present technology comprises the following aspects: an interfacing structure 1100, a lens 1200, a positioning and stabilizing structure 1300 (for example, an elasticised strap), and a lens frame 1400. In some forms, a functional aspect may provide one or more physical components. In use, the goggles are arranged to be positioned proximate and anterior to the user's eyes, so as to allow the user to see through the lens 1200.

Referring to FIG. 1C, the lens frame 1400 comprises a forehead portion 1402 crossing the sagittal plane of the user's head in use, a pair of lateral portions 1404, a pair of cheek portions 1406 provided on respective sides of the user's nose in use, and a nasal portion 1408 bridging between the cheek portions 1406. In examples, the lens frame may include one or more vents 1410 providing an air pathway between an interior space defined by the goggles 1000 and user 100 in use, and an exterior. In examples a baffle material may be provided in the vents 1410 (for example a foam material), or a tortuous pathway.

Referring to FIG. 1D, the interfacing structure 1100 comprises a forehead portion 1102 crossing the sagittal plane of the user's head in use, a pair of lateral portions 1104, a pair of cheek portions 1106 provided on respective sides of the user's nose in use, and a nasal portion 1108 bridging between the cheek portions 1406.

Referring to FIGS. 1I and 1K the interfacing structure 1100 may comprise a face engaging portion having a face engaging flange 1120 configured to engage the user's face in use. The face engaging flange 1120 may be flexible and resilient. The face engaging flange 1120 may be formed at least partially from an elastomeric material, such as silicone or a TPE, for example.

Referring to FIGS. 1J and 1K the interfacing structure 1100 may comprise a cushion 1130. In some examples the one or more cushions are formed from foam. For example, the face engaging flange 1120 may cover at least a portion of a foam cushion located within, as described in International (PCT) Application No. PCT/AU2021/050277, the entirety of which is hereby incorporated by reference herein. The face engaging flange 1120 extends from a radially outward position to a radially inward position, curving over at least a portion of the cushion 1130 from the radially outward position.

In the example shown in FIGS. 1B-1H, the cushion 1130 is formed in two parts. In alternative examples the cushion 1130 is formed as a unitary part.

In the example shown in FIGS. 1B-1H, the face engaging flange 1120 is secured to the lens frame 1400 using an adhesive. In alternative examples the interfacing structure 1100 comprises a chassis portion, the chassis portion being stiffer than the face engaging flange 1120, wherein the face engaging flange 1120 is attached to and supported by the chassis portion. The chassis portion may then be connected to lens frame 1400. In alternative examples the goggles 1000 may not include a lens frame 1400—i.e. the interfacing structure 1100 or chassis portion may be provided directly to the lens 1200.

In the example shown in FIGS. 1B-1H, the nasal portion 1108 comprises foam material which is not covered by the face engaging flange 1120. In this example, lateral nasal portions 1110 of the face engaging flange 1120 extend partially over the foam of the nasal portion 1108. In alternative examples, the face engaging flange 1120 may cover the span of the nasal portion 1108.

Referring to FIGS. 4A to 4I, according to this aspect of the present technology the interfacing structure 1100 connects to the lens 1200. In this aspect of the technology the interfacing structure 1100 comprises a compliance portion 1150 comprising a support flange 1154 around a periphery of an eye region of the user's face, and a cushion support flange 1160 extending from the support flange 1154, the support flange 1154 being flexible and resilient (for example, made of silicone). A cushion 1130 (for example, made of foam) configured to engage the user's face in use is provided to the cushion support flange 1160 to provide a contact zone to the user. In use, the cushion 1130 adapts to small-scale facial shape variation, while the compliance portion 1150 adapts to large-scale facial shape variation.

In this example, with particular reference to FIG. 4G and FIG. 4H, a chassis portion 1140 connects a compliance portion 1150 to the lens 1200. This chassis portion 1140 has a relatively high stiffness to enable connection to lens 1200, and handling of the flexible compliance portion 1150 during manufacture. In alternative examples the compliance portion 1150 could be directly moulded to the lens 1200.

FIG. 4J shows an alternative example for the interfacing structure 1100 in which the compliance portion 1150 comprises a relatively thicker portion 1156 at the base proximal the lens 1200.

FIG. 4K shows an alternative example for the interfacing structure 1100 in which the support flange 1154 connects to the cushion support flange 1160 at an intermediary position. In the example of FIG. 4K the support flange 1154 connects to a periphery of the lens 1200.

FIG. 4L shows an alternative example for the interfacing structure 1100 in which the cushion support flange 1160 extends only partially beneath the cushion 1130.

FIG. 4M shows an alternative example for the interfacing structure 1100 in which the support flange 1154 comprises a flexure portion 1158 in the form of a concertina section.

FIG. 5A shows a top cross-sectional view comparing field of view of goggles according to an aspect of the present technology with goggles of a typical construction which utilises a foam cushion 110 in place of the interfacing structure 1100 of the present technology. In this example, field of view (FOV) is improved in comparison with traditional goggle construction using a comfortable material and geometry combination that is “low profile” i.e., takes less space in dimensions that impact FOV. Typically, solutions containing foam connect it to a relatively firm “base” that does not comply, so the foam does all the necessary compliance or movement on its own. Large amounts of compliance require “tall” or high aspect cross sections that in foam material can be unstable to lateral forces and difficult to achieve reliable, predictable performance of the material and geometry.

Referring to FIG. 5B, the support flange 1154 provides compliance and stability simultaneously. The interfacing structure 1100 is configured to interfere with the face so as to flex outwards upon fit.

In examples, as shown in FIG. 6A, the lens 1200 extends laterally beyond the periphery of the interfacing structure 1100. In alternative examples, as shown in FIG. 6B, the lens 1200 may be contained within the periphery of the interfacing structure 1100.

FIG. 7A to 7B interfacing structure comprising a flexible and resilient compliance portion 1150 supporting a foam cushion 1130. Referring to FIG. 8A to 8C, the cushion 1130 may include a rounded leading edge (i.e., facing in the posterior direction), to reduce drag when fitting to the nose.

Referring to the alternative example shown in FIG. 8D, the thickened portion 1156 of the support flange 1154 resists outwards movement of the contact zone on the face of the cushion 1130 with bending-beam behaviour, providing enough resistance to maintain contact with face, while being compliant to anthropometric variation. The flexure portion, in the form of a localised thin zone, promotes pivoting so the contact zone is “self-levelling”. The cushion support flange 1160 enables the contact zone to maintain its relative flat shape, resulting in even contact pressure for improved comfort.

In the example of FIG. 8E, the inner end of the cushion support flange 1160 is canted towards centreline. The shape may be moulded to accommodate nasal proportions of different ethnic groups. This design pivots passively at the junction between the support flange 1154 and the cushion support flange 1160 to fit each nose type: clockwise for shorter/wider/less-projecting shapes, anti-clockwise for narrower/more-projecting shapes.

In the example of FIG. 8F, the interfacing structure 1100 flexes clockwise to better fit shorter/wider/less-projecting nose shapes.

In the example of FIG. 8G, a longer support flange 1154 allows for a high degree of compliance movement, while the cushion support flange 1160 is pre-loaded towards centreline.

FIG. 8H illustrates how the shape of the nasal portion may vary—for example, in a key-hole manufactured shape to increase pre-load contact at sides and nose bridge of the user—e.g., biased towards a first shape 1109a, and capable of being deformed to a second shape 1109b.

Referring to FIG. 8I and FIG. 8J, in examples the compliance portion 1150 comprises an anterior nasal flange portion 1151 in the nasal portion 1108, comprising a thickened portion 1156 proximal the sides of the user's nose in use, and a flexure portion 1158 proximal the ala crest, wherein the cross-sectional thickness of the thickened portion 1156 is greater than that of the flexure portion 1158. The thickened portion 1156 is configured to control lateral movement and resistance. The flexure portion 1158 is configured to block air/moisture ingress while allowing the compliance zone to flex unhindered by this element.

Referring to FIG. 9A-1 to FIG. 9A-3, in examples of the present technology the interfacing structure 1100 comprises a compliance portion 1150 and a cushion portion 1130 provided to the compliance portion 1150. In this example the compliance portion 1150 is made of a flexible elastomer such as silicone. In examples the compliance portion 1150 may be overmoulded to the lens 1200. In examples the cushion 1130 is made of foam, having a cover layer 1132 (for example, fleece, or textile, or flocking). In examples the foam cushion 1130 may be cut in two-dimensions (2D), and assume a three-dimensional (3D) shape when assembled to the silicone compliance portion 1150. FIG. 9B-1 to 9B-3 illustrates a similar configuration, without the cover layer 1132.

Referring to FIG. 9C-1 to FIG. 9C-3, in examples a cover layer 1132 may be provided directly to the compliance portion 1150 to provide a contact zone. In such examples the cover layer 1132 may be a material such as a fleece or high-thickness textile, or composite such as a first layer 1134 as a core and a second layer 1136 as a spacer.

Referring to FIG. 9D-1 to FIG. 9D-3 and FIG. 9E-1 to FIG. 9E-3, in examples the compliance portion 1150 may be made of a foam. In these examples, the foam compliance portion 1150 may be compression cut to shape and assume a 3D shape when assembled to the lens 1200. In the example of FIG. 9E-3, the radially inward surface of the foam compliance portion 1150 may be shaped to follow the curvature of the radially outward surface. In examples, the compliance portion 1150 may be provided to a carrier member 1170, and the carrier member 1170 attached to the lens 1200. In examples a cover layer 1132 may be provided directly to the compliance portion 1150 to provide a contact zone. In such examples the cover layer 1132 may be a material such as a fleece or textile.

Referring to FIG. 9F-1 to FIG. 9F-3, in examples the compliance portion 1150 and the cushion 1130 may be made of different density foam (such as an injection moulded foam), such that the compliance portion 1150 is made of foam having a first density, and the cushion 1130 is made of foam having a second density lower than the first density. Referring to FIG. 9G-1 to FIG. 10G-3, in examples the compliance portion 1150 may be made of a foamed silicone.

Referring to FIG. 10A and FIG. 10A-1, in examples of the present technology the interfacing structure 1100 comprises a compliance portion 1150 and a cushion portion 1130 provided to the compliance portion 1150. In this example the compliance portion 1150 comprises a lens connection portion 1152, configured to be secured relative to the lens 1200. A support flange 1154 extends in a posterior direction from the lens connection portion 1152. The support flange 1154 includes a thickened portion 1156 proximal the lens connection portion 1152, tapering off into a flexure portion 1158. The support flange 1154 is curved, and provides a compression zone with a wall thickness—particularly in the thickened portion 1156—that increases to increase the stiffness the more it is compressed. Generally, deeper walls (i.e. in the anterior-posterior direction) provide more compliance. Curvature also affects stiffness, with more shallow curves being firmer and taller curves being softer.

A cushion support flange 1160 extends in a radially inward direction from the flexure portion 1158. The flexure portion 1158 assists in controlling the stiffness of the cushion support flange 1160 as it flexes away from the face. A thin flexure portion 1158 ensures the cushion support flange 1160 will flex more easily to match the surface of the face without adding much stiffness.

The cushion support flange 1160 has a flat profile to attach a cushion 1130 to, and roughly defines the surface that conforms to the face. A relatively greater thickness resists stretching along the profile, and can encourage geometry changes between zones of the interfacing structure 1100 to match large scale variation in face curvature. It also provides more stable surface for cushion attachment.

A cushion support flange angle 1162 between the cushion support flange 1160 and a horizontal plane defines the presentation of the cushion support flange 1160 to the face. In examples, this angle may be biased towards the face (i.e. biased in a posterior direction) in areas with higher local face variation in order to assist in providing additional low-stiffness compliance. Referring to FIG. 10B, in examples the forehead portion 1102 and cheek portions 1106 of the interfacing structure 1100 may be generally configured to register to the face and transfer the majority of the sealing force (i.e. from the positioning and stabilising structure 1300) to the face to provide stability. In examples the lateral portions 1104 and nasal portion 1108 of the interfacing structure 1100 may be generally configured to sit relatively lightly on the face, enough to seal, and be capable of adapting to large variations in face shape.

Referring to FIG. 10C, the profile of the compliance portion 1150 may vary between zones. For example, the forehead portion 1102 may have a first compliance portion profile 1150a, the lateral portions 1104 may have a second compliance portion profile 1150b, the cheek portions 1106 may have a third compliance portion profile 1150c, and the nasal portion 1108 may have a fourth compliance portion profile 1150d.

Referring to the first compliance portion profile 1150a in FIG. 10D, the relatively large size of fit zone (e.g., in comparison with third compliance portion profile 1150c) means as high a local stiffness not required, so support flange 1154 thickness is lower (e.g., in comparison with third compliance portion profile 1150c), but the curve is more shallow to provide some stiffness. The cushion support flange angle 1162 is similar to face angle to encourage deformation, which will encourage compliance at the temples in narrow heads.

Referring to the second compliance portion profile 1150b in FIG. 10E, the support flange 1154 curvature is shallow but deep, and highly offset from the end of the wall to allow hinging, so the compliance portion 1150 can comply with a range of head widths. The cushion support flange angle 1162 is slightly biased toward the face to provide extra compliance, and cushion support flange 1160 slightly longer (e.g., in comparison with the first compliance portion profile 1150a) to increase contact area.

Referring to the third compliance portion profile 1150c in FIG. 10F, there is limited space between the lens 1200 and face in this region, which requires a shorter support flange 1154 (e.g., in comparison with first compliance portion profile 1150a, and/or second compliance portion profile 1150b), but the cushion support flange angle 1162 is higher (e.g., in comparison with first compliance portion profile 1150a and/or second compliance portion profile 1150b) to assist with contact with the face despite cheekbone variation. The cushion support flange 1160 is also shorter (e.g., in comparison with the first compliance portion profile 1150a) to stay away from sensitive eye area.

Referring to the fourth compliance portion profile 1150d in FIG. 10G, larger face variation in the zone leads to a deep, thin support flange 1154 as well as a high cushion support flange angle 1162 (e.g., in comparison with first compliance portion profile 1150a and/or second compliance portion profile 1150b) to assist with achieving contact on a wide range of nose shapes, without excessive pressure. Cushion support flange 1160 thickness has been reduced (e.g., in comparison with the first compliance portion profile 1150a, second compliance portion profile 1150b, and/or third compliance portion profile 1150c) to reduce its resistance to stretching and allow more compliance.

FIG. 11A to FIG. 11D show examples of means for securing the interfacing structure 1100, more particularly compliance portion 1150, to the lens 1200. In FIG. 11A, the compliance portion 1150 comprises an overmould feature 1164, with the compliance portion 1150 overmoulded to the lens 1200 during manufacture. In FIG. 11B, an adhesive 1166 is provided between the compliance portion 1150 and the lens 1200. In FIG. 11C, a mechanical interlock 1168 is provided between the compliance portion 1150 and the lens 1200. In FIG. 11D, a carrier member 1170 may be overmoulded to the compliance portion 1150, and the carrier member 1170 attached to the lens 1200 (e.g. using an adhesive tape).

According to aspects of the present technology at least a portion of the positioning and stabilising structure 1300 may be integral with the interfacing structure 1100. For example, as shown in FIG. 12A and FIG. 12B, the goggles 1000 may comprise integrated strap portions 1302 extending from lateral portions of the compliance portion 1150. In the example shown, the integrated strap portions 1302 are configured to be secured to each other using a fastener 1304 (e.g. hook and latch material, clips, magnetic fasteners or other suitable means). In alternative examples the integrated strap portions 1302 may be replaced by a continuous strap. In examples the integrated strap portions 1302 may comprise rigidised portions 1306 to assist with vector control and provide a pre-formed shape. This may assist with improving fit to the head, or improve usability by making the strap portions 1302 easier to control (i.e. less floppy to handle).

In examples, the integrated strap portions 1302 may be constructed from: an elastomer; elastomer with textile on skin contact surface; or composite laminations (e.g. comprising combinations of foam, textile, elastomer, and/or semi-rigid thermoplastic).

In examples the goggles 1000 may comprise an integral strap connection feature to which a positioning and stabilising structure 1300 in the form of a strap may be secured. Referring to FIG. 12C, a strap connection extension 1310 may be integral with lateral portions of the compliance portion 1150. A strap connection feature 1312 is provided to the strap connection extension 1310, to which a strap 1300 may be secured. For example, the strap connection feature 1312 may have an opening through which the strap 1300 can be passed and then looped back and secured onto itself. In alternative arrangements the strap connection feature 1312 may comprise magnetic fasteners and or other mechanical fastening mechanisms. In examples the strap 1300 may be pivotally connected to the strap connection extension 1310.

In examples (not illustrated), strap connection extension 1310 and optionally a strap connection feature 1312 may be provided to the lens 1200 or an intermediary component such as lens frame 1400. In examples the strap 1300 may be pivotally connected to the strap connection extension 1310, and/or the strap connection extension 1310 may be pivotally connected to the lens 1200 or intermediary component. In examples the strap connection extension 1310 may be configured to flex relative to the lens 1200 or intermediary component.

According to aspects of the present technology the positioning and stabilising structure 1300 may include vents in the compliance portion 1150 providing an air pathway between an interior space defined by the goggles 1000 and user 100 in use, and an exterior. Referring to FIG. 13A and FIG. 13B, the support flange 1154 of the compliance portion 1150 may comprise openings spanned by hydrophilic vent portions 1180. For example, the hydrophilic vent portions 1180 may comprise a hydrophilic textile material configured to prevent moisture ingress while allowing ventilation to reduce the likelihood of fogging. In examples, a foam vent feature 1182 may be provided on an external surface of the hydrophilic vent portions 1180 to reduce the likelihood of snow and/or ice build up. In examples, as show in FIG. 13B, reinforcement portions 1184 in the support flange 1154 may assist with maintaining anthropometric fit and contact force at the venting locations.

FIG. 14A shows an exemplary interfacing structure 1100 in which the nasal portion 1108 comprises a compliance portion 1150, and a cushion portion 1130 provided to the compliance portion. In this example the compliance portion 1150 and the cushion portion 1130 are continuous through the nasal portion 1108, including across the user's nasal bridge in use. The nasal portion 1108 is configured to adapt to different nose shapes primarily through flexing, bending, and/or rolling of the compliance portion 1150. In examples the cushion portion 1130 comprises a layer of foam and a textile layer configured to contact the user in use.

Referring to FIG. 14B, in examples the compliance portion 1150 comprises an adaptation feature 1500 in the form of a collapsing feature (e.g. a ‘S’ shaped fold) in its profile within the nasal portion 1108 to allow the contact portion of the interfacing structure 1100 (e.g. cushion portion 1130) to translate forward to accommodate a change in nose size/shape. In examples, the collapsing feature has maximum dog-leg length at the centreline of the interfacing structure 1100 (i.e. where the nose bridge locates) and tapers to a shorter length either side of the centreline (where the sides of the nose locates) until it ends at the cheek regions. In examples, the cushion portion 1130 provided to the compliance portion in the nasal portion 1108 is thinner in cross-section than other areas (for example the forehead portion). In examples the cushion portion 1130 may be capable of stretching to a greater extent, to accommodate movement of the collapsing feature.

Referring to FIG. 14C and FIG. 14D, in examples the compliance portion 1150 may have an intermediary adaptation feature 1500 having excess length configured to allow a nasal bridge contact portion 1159a in the nasal portion 1108 to move in a superior direction in response to changes in nose size and/or shape. In examples the contact point in the nasal portion 1108 may be provided on a cushion support flange 1160 configured to pivot relative to the support flange 1154 of the compliance portion 1150. In examples the compliance portion 1150 may comprise an inferior portion 1159b and the nasal bridge contact portion 1159a, with the adaptation feature 1500 extending between the inferior portion and the nasal bridge contact portion. In examples the compliance portion 1150 may comprise a junction between the inferior portion 1159b and the nasal bridge contact portion 1159a on either side of the nasal portion 1108.

Referring to FIG. 14E, in examples the junction may comprise a living hinge feature 1148. In examples the living hinge feature 1148 may be provided by providing the profile with excess length (e.g., approximating a “μ”) between the branches (e.g., as shown in FIG. 14E-1). In examples the living hinge feature 1148 may be provided by a bellows or concertina type corrugation (e.g., as shown in FIG. 14E-2).

Referring to FIGS. 14F1 to 14F2-2, and FIG. 14G and FIG. 14G-1, in examples the nasal portion 1108 of the compliance portion 1150 comprises posterior flaps 1111 disposed to either side of the user's nose in use. The posterior flaps 1111 are configured to sit on a lower profile nose, and flex in an anterior direction to accommodate a higher profile nose.

FIG. 15A shows an exemplary interfacing structure 1100 in which the nasal portion 1108 is segmented into lateral nasal portions 1110 such that at least the cushion portion 1130 is not continuous (i.e., a single piece) across the user's nasal bridge in use. The nasal portion 1108 is configured to adapt to different nose shapes primarily through flexing and/or bending of the compliance portion 1150.

Referring to FIG. 15B, in examples a slot 1530 may be provided in the posterior edge of the compliance portion 1150 proximal the centreline of the nasal portion 1108. In examples, e.g., as shown in FIG. 15B-1, a pocket 1532 may be provided at the closed end of the slot 1530. In examples, e.g., as shown in FIG. 15B-2, reinforcing 1534 may be provided to lateral nasal portions 1110 on either side of the slot 1530, maintaining force on the user's nose in use. In examples, e.g., as shown in FIG. 15B-3, the closed end of the slot 1530 may expand to form a “keyhole”. The keyhole may assist with keeping the edges of the slot 1530 closer to parallel when forced apart by the user's nose rather than splaying.

Referring to FIG. 15C, in examples the compliance portion 1150 at the nasal portion 1108 comprises at least one flexure portion 1158 permitting greater movement in the compliance portion 1150 to adapt to nose shape/size. In the illustrated example the flexure portion 1158 is one or more slots, but in alternative examples the flexure feature may comprise regions of reduced cross-sectional thickness. In this example, the cushion portion 1130 at the nasal portion 1108 may include a nasal bridge portion 1138 separate to the remainder of the cushion portion 1130. In the example illustrated, the nasal bridge portion 1138 overlaps adjacent portions of the cushion portion 1130 (for example, using matching bevelled edges) such that relative movement does not open a direct air path through the join.

Referring to FIG. 15D-1 and FIG. 15D-2, in examples the nasal portion 1108 of the compliance portion 1150 includes an adaptation portion 1500 between lateral nasal portions 1110, the adaptation portion 1500 providing excess length to allow the lateral nasal portions 1110 to separate laterally rather than splaying in response to contact from larger noses, e.g., from a narrow position as shown in FIG. 15D-1 to a wider position as shown in FIG. 15D-2.

Referring to FIG. 15E, in examples the nasal portion 1108 may comprise textile flaps 1113 configured to lay on the user's nose in use. In examples the textile flaps 1113 may be configured to provide a gap between the flaps 1113 at the user's bridge, while in alternative examples the flaps 1113 may overlap (e.g., as shown in FIG. 15E-1).

FIG. 16A shows an exemplary interfacing structure 1100 in which the nasal portion 1108 is configured to adapt to nose size/shape independently of cheek portions 1106. The nasal portion 1108 is configured to adapt to different nose shapes primarily through rolling, rotating and/or bending of the compliance portion 1150. Examples of such a nasal portion 1108 may or may not include cushion portion 1130.

Referring to FIG. 16B and FIG. 16B-1, and FIG. 16C-1 and FIG. 16C-2, in examples the nasal portion 1108 may be configured to rotate in the sagittal plane to adapt to nose size/shape. In examples this may be integral with the remainder of the interfacing structure 1100, while in alternative examples this region may be formed separately and connected to the remainder of the interfacing structure 1100.

Referring to FIG. 16D, in examples the nasal portion 1108 of the compliance portion 1150 is concave in a radially inward direction. This allows the compliance portion 1150 to “roll” inwardly under contact with the user's nose, to adapt to differences in size/shape.

Referring to FIG. 16E, in examples the nasal portion 1108 of the compliance portion 1150 is configured to extend in an anterior direction from the lens before curving around in a posterior direction. This allows the compliance portion 1150 to “roll” outwardly under contact with the user's nose, to adapt to differences in size/shape.

Referring to FIG. 16F, in examples the nasal portion 1108 may be configured to be modular—i.e., different nose modules 1112a to 1112c (as shown in FIGS. 16F-1 to 16F-3) may be selectively attached, each nose module 1112 configured to suit a different nose size/shape.

FIGS. 17A and 17B shows exemplary goggles 1000 comprising an interfacing structure 1100 according to an aspect of the present technology. Referring to FIGS. 17C and 17D, in examples the compliance portion 1150 in the nasal portion 1108 of the interfacing structure 1100 comprises an adaptation portion 1500 between the lens 1200 (see FIG. 17B) and a posterior nasal contact portion 1502 of the compliance portion 1150. In this example the adaptation portion 1500 has a concave cross-section facing in the inferior direction, enabling the compliance portion 1150 to “roll” under contact with the user's nose, to adapt to differences in size/shape.

FIGS. 18A and 18B shows exemplary goggles 1000 comprising an interfacing structure 1100 according to an aspect of the present technology. Referring to FIGS. 18C to 18L, in examples the compliance portion 1150 in the nasal portion 1108 of the interfacing structure 1100 comprises an intermediary portion 1504 between an adaptation portion 1500 and a posterior nasal contact portion 1502 of the compliance portion 1150. In this example the adaptation portion 1500 has a concave cross-section facing in a radially outwards direction. In this example, the cross-sectional thickness of the intermediary portion 1504 is greater than that of the adaptation portion 1500. In this example the posterior nasal contact portion 1502 curves in a radially inward direction from the intermediary portion 1504. Referring to FIGS. 18F to 18H, under contact with the user's nose the adaptation portion 1500 deforms to allow the intermediary portion 1504 and posterior nasal contact portion 1502 to rotate about an inferior rotation or pivot point to adapt to differences in size/shape. Further, the posterior nasal contact portion 1502 bends in response to contact with the user's nose.

FIGS. 19A to 19C-3 show an exemplary interfacing structure 1100 having a similar configuration to that described above with reference to FIGS. 18A to 18L. In this example, the compliance portion 1150 in the nasal portion 1108 of the interfacing structure 1100 comprises lug portions 1506 extending laterally from the intermediary portion 1504. The lug portions 1506 are provided at an inferior position, proximal the transition between the nasal portion 1108 and the cheek portions 1106. In this example, a support member 1510 made of a more rigid material than the compliance portion 1150 (for example, a thermoplastic) includes a bridging portion 1512 and lug locating features 1514 disposed at each end of the bridging portion. The bridging portion 1512 spans over the nasal portion 1108, against the lens connection portion 1152. The lug locating features 1514 secure to the lug portions 1506, leaving an unconstrained portion 1508 of the lug 1506 between the lug locating features 1514 and the intermediary portion 1504. The unconstrained portion 1508 of the lugs 1506 can twist, adding resistance to overall rotation of the intermediary portion 1504 and posterior nasal contact portion 1502.

FIG. 20A shows exemplary goggles 1000 comprising a lens 1200 provided to an interfacing structure 1100 according to an aspect of the present technology. In this example, a cover portion 1190 is provided to radially outward surfaces of the compliance portion 1150. In examples, the cover portion 1190 may be made of a textile, or a foam, material. As an example, the textile may be made of polyethersulfone (PES)—for example a PES piquet material. In examples the cover portion 1190 may be configured to resist passage of water through the cover portion—for example made of a hydrophobized material (e.g. a hydrophobic textile).

Referring to FIG. 20B, the forehead portion 1102 of the compliance portion 1150 comprises a plurality of vents 1410 between radially inward and radially outward surfaces of the compliance portion 1150. In this example, the vents 1410 are circular in shape. In alternative examples, the vents 1410 may be other geometric shapes—for example triangles (e.g. in a repeating pattern facing in alternating directions—see FIG. 27C)—or forms, such as linear shapes. In examples, the vents 1410 are disposed towards the anterior of the compliance portion. In examples the size of each vent 1410 is greatest proximal the centreline, and the size of each vent 1410 reduces in size laterally away from the centreline. As shown in FIG. 20C, in examples vents 1410 are provided proximal the transition between the lateral portions 1104 and the cheek portions 1106 of the compliance portion 1150. In the example illustrated in FIGS. 20A to 20E the cover portion 1190 extends continuously over all of the vents 1410. In alternative examples a plurality of discrete cover portions 1190 may be provided to different groups of vents 1410.

Referring to FIG. 20B, in examples the compliance portion 1150 comprises a reinforcement portion 1184 in the form of a rib having a greater cross-sectional thickness. The reinforcement portion 1184 extends between laterally disposed points on the anterior edge of the compliance portion 1150 (i.e. lens connection portion 1152), offset in a posterior direction from the anterior edge when intersecting the centreline. It is envisaged that the reinforcement portion 1184 may increase resistance to compression of the forehead portion 1102 when the goggles 1000 are fit to the user's face. In this example, the majority of vents 1410 in the forehead portion 1102 are provided within the bounds of the anterior edge and the reinforcement portion 1184.

Referring to FIG. 20D and FIG. 20E, in the illustrated example the posterior edges of the foam cushion 1130 are rounded. In alternative examples these edges may be squared.

FIGS. 21A to 21E-1 show an exemplary interfacing structure 1100 according to an aspect of the present technology. In this example, the support flange 1154 of the compliance portion 1150 at the forehead portion 1102 comprises an anterior support flange portion 1155a and a posterior support flange portion 1155b. Referring to FIG. 21 D, the posterior support flange portion 1155b extends in an inferior-posterior direction from the anterior support flange portion 1155a. In the illustrated example, the cushion support flange 1160 comprises a radially outward cushion support flange portion 1161a extending radially outward from the posterior support flange portion 1155b, and a radially inward cushion support flange portion 1161b extending radially inward from the posterior support flange portion 1155b. In alternative examples, the cushion support flange 1160 may not comprise a radially outward cushion support flange portion 1161a, and the foam cushion 1130 shaped accordingly.

Referring to FIGS. 21B to 21E-1, in examples the compliance portion 1150 in the nasal portion 1108 of the interfacing structure 1100 comprises an adaptation portion 1500 between the lens connection portion 1152 and the posterior nasal contact portion 1502 of the compliance portion 1150. In this example the adaptation portion 1500 comprises a bridge adaptation portion 1516 extending along the lens connection portion 1152 over the bridge of the user's nose in use, and lateral adaptation portions 1518 extending from the bridge adaptation portion 1516 in a posterior direction towards a posterior edge of the posterior nasal contact portion 1502. The adaptation portion 1500 assists with adaptation to nose size/shape by translation/rotation of the posterior nasal contact portion 1502, with pivoting of the posterior nasal contact portion 1502 in response to different user nose size/shape occurs at a curved transition adaptation portion 1520 between the bridge adaptation portion 1516 and the lateral adaptation portions 1518, and extension in the superior-inferior directions occurring via the lateral adaptation portions 1518. In examples, the bridge adaptation portion 1516 has a concave cross-section facing in the anterior-inferior direction (see FIG. 21D), and the lateral adaptation portions 1518 have a concave cross-section facing in the posterior direction.

Referring to FIG. 21D, FIG. 21E, and FIG. 21E-1, in examples an anterior nasal contact portion 1503 extends in an anterior direction from the adaptation portion 1500, increasing the contact area with the user's nose in use in comparison with alternative examples comprising the posterior nasal contact portion 1502 only. FIG. 21E illustrates the interfacing structure 1100 in a manufactured state. In use, the adaptation portion 1500/posterior nasal contact portion 1502/anterior nasal contact portion 1503 flexes apart to fit to the user.

FIG. 22A and FIG. 22B show exemplary goggles 1000 comprising a lens 1200 provided to an interfacing structure 1100 according to an aspect of the present technology. In this example, strap connection features 1312 are provided to each lateral side of the lens 1200. Each strap connection feature 1312 comprises a pivot feature 1314, about which the strap connection feature 1312 pivot relative to the lens 1200. In examples, the pivot feature 1314 comprises an aperture through which a locator such as a pin or bolt passes to locate the strap connection feature 1312 relative to the lens 1200. In examples, the strap connection feature 1312 comprises a strap connection receptacle 1316, configured to receive and engage with a component at an end of a strap.

FIGS. 23A to 23D show an exemplary interfacing structure 1100 according to an aspect of the present technology. In examples, the compliance portion 1150 comprises anterior radially outward flange portion 1153 extending from the lens connection portion 1152. In examples, the anterior radially outward flange portion 1153 is provided in the forehead portion 1102 and lateral portions 1104 of the compliance portion 1150. The anterior radially outward flange portion 1153 allows increased contact area for connection with a lens 1200 (see FIG. 23E), and increased space for alternate strap attachment methods (not shown).

Referring to FIG. 23C, in examples a plurality of vents 1410 are provided in the forehead portion 1102 of the compliance portion 1150, disposed towards the lens 1200. In examples, a hydrophobic textile cover portion 1190 is provided over the vents 1410 in the forehead portion 1102. Referring to FIG. 23D, in examples vents 1410 may be provided in the compliance portion 1150 in locations shielded by the anterior radially outward flange portion 1153—for example at the transition between the lateral portions 1104 and the cheek portions 1106. In examples, the vents 1410 may have linear shapes—for example a ‘V’ shaped slot—offering increased resistance to snow ingress for similar flow area as an equivalent array of circular vents.

Referring to FIG. 23E, in examples strap connection features 1312 are pivotally secured to the lens 1200. In this example, the strap connection feature 1312 comprises a strap opening 1313 through which a strap may be looped to secure the strap relative to the lens 1200 and interfacing structure 1100.

Referring to FIGS. 24A and 24B, in examples it may be desirable to reduce the visual gap between a superior lens edge 1202 of the lens 1200 and a rim 1602 of a helmet 1600 (not illustrated in FIG. 24A or FIG. 24B, but see FIG. 25C) worn by a user. However, it may also desirable to maintain a cross-sectional shape in the forehead portion compliance portion 1150 that responds effectively under compliance load in use, without buckling. FIGS. 24C to 24G illustrate alternative examples of interfacing structures 1100. In the example of FIG. 24C, the support flange 1154 extends in an inferior-posterior direction from the superior lens edge 1202. In this example the support flange 1154 has a concave shape facing in the inferior direction. In the example of FIG. 24D, the support flange 1154 extends in a superior-posterior direction from the superior lens edge 1202 and has a concave shape facing in the inferior direction. In this example, a textile cover portion 1190 is provided over the support flange 1154 and cushion 1130. In the example of FIG. 24E the support flange 1154 first extends in a posterior direction from the superior lens edge 1202, and then in a superior-posterior direction. In this example, the support flange 1154 flexes radially inwardly under compliance load. In this example, a textile cover portion 1190 is provided over the support flange 1154 and cushion 1130. In the example of FIG. 24F, the support flange 1154 first extends in a posterior direction from the superior lens edge 1202, and then in an inferior-posterior direction. In this example, the first portion of the support flange 1154 has a concave shape facing in the superior direction, such that under compliance load the first portion flexes radially inwardly, and the second portion flexes radially outwardly. In this example, the support flange 1154 at the lens 1200 is shaped to receive an edge of the textile cover portion 1190. In the example of FIG. 24G the support flange 1154 first extends in a posterior direction from the superior lens edge 1202, and then in an inferior-posterior direction. In this example, the support flange 1154 flexes radially outwardly under compliance load.

FIG. 25A shows an exemplary positioning and stabilising structure 1300 according to an aspect of the present technology. In examples the positioning and stabilising structure 1300 comprises a pair of lateral strap portions 1320 configured to be secured to goggles 1000 (for example via strap connection feature 1312) and lie on each side of the user's head in use. In examples the lateral strap portions 1320 are capable of stretching (for example being made of elasticised material). In examples the lateral strap portions 1320 connect to rigidised portions 1306. In examples the rigidised portions 1306 are made of a form holding material—for example a textile/foam laminate—and are configured to assist with vector control and provide a pre-formed shape. In examples, the rigidised portions 1306 are configured to extend in a superior-posterior direction from the lateral strap portions 1320 in order to direct the force vector up and over the ear of a user.

In examples, the positioning and stabilising structure 1300 comprises a superior posterior strap portion 1330 configured to extend in a superior-posterior direction from the rigidised portions 1306 around the back of the user's head in use. In examples, the positioning and stabilising structure 1300 comprises an inferior posterior strap portion 1340 configured to extend in a posterior direction from the rigidised portions 1306, inferior to the superior posterior strap portion 1330, around the back of the user's head in use. In examples, the length of one or both of the superior posterior strap portion 1330 and the inferior posterior strap portion 1340 may be adjustable—for example using slide buckles 1350.

In use, when goggles 1000 comprising the positioning and stabilising structure 1300 are worn by the user—whether without a helmet (see FIG. 25B) or with a helmet (see FIG. 25C)—stretch in the lateral strap portions 1206 provides flexibility in fitting to the user's head, headwear, or helmet. In examples, the position of the connection to the lens 1200 or interfacing structure 1100 may be selected to provide a force vector straight back in a posterior direction. In alternative examples, the position of the connection to the lens 1200 or interfacing structure 1100 may be selected to bias more force toward the forehead than the cheeks of the user.

FIGS. 26A to 26F show an exemplary vent valve 1420 provided to vents 1410 in a compliance portion 1150 of an interfacing structure 1100 according to an aspect of the present technology. In examples, the vent valve 1420 includes a flap portion 1422 structured and arranged to regulate flow through the vents 1410 (see for example, vents 1410 in FIG. 20B, FIG. 20C, 21A). The flap portion 1422 is connected to the compliance portion 1150 on an anterior side of the vents 1410, and extends over the vents 1410 in the anterior to posterior direction.

In examples, the flap portion 1422 is biased or pre-loaded to an open position (see, FIG. 26B). In examples, in the open position the flap portion 1422 extends at an acute angle relative to the portion of the compliance portion 1150 over which it extends. When airflow in the anterior to posterior direction is below a threshold, the flap portion 1422 remains in the open position and permits flow between the compliance portion 1150 and flap portion 1422 along the posterior edge and through the vents 1410. As the airflow in the anterior to posterior direction increases, the flap portion 1422 closes to cover the vents 1410 (see, FIG. 26C).

In examples, the flap portion 1422 is configured to close proportional to the relative speed of airflow in the anterior to posterior direction. In examples the flap portion 1422 may be maintained at one or more positions between the fully open position of FIG. 26B and the fully closed position of FIG. 26C, depending on the relative speed of airflow in the anterior to posterior direction (see, e.g. FIG. 26D).

In examples, vent valve 1420 may be configured to maintain a gap between the flap portion 1422 and the compliance portion 1150 when in a closed position. In the example of FIG. 26E, one or more vent valve spacer portions 1424 are provided on the radially outward facing surface of the compliance portion 1150 on the posterior side of the vents 1410. In such examples the one or more vent valve spacer portions 1424 act as stops to prevent complete sealing of the vents 1410 by the flap portion 1422. The resulting air gaps to either side of the one or more vent valve spacer portions 1424 allow for a minimum level of airflow through the vents 1410.

Referring to FIG. 26F, in examples the flap portion 1422 may include a secondary vent 1426 which at least partially overlaps a vent 1410 when the flap portion 1422 is in the closed position. The area A₁ of the vent 1410 is greater than the area A₂ of the secondary vent 1426, such that the effective area of the vent 1410 is delimited by secondary vent 1426 when the flap portion 1422 is in the closed position.

In examples, a vent valve 1420 may be provided to one or more, but not all, vents 1410 in the compliance portion 1150. For example, a vent valve 1420 may be provided to vents 1410 proximal the transition between the lateral portions 1104 and the cheek portions 1106, but not those in the forehead portion 1102. In an alternative embodiment, a vent valve 1420 may be provided to vents 1410 in the forehead portion 1102, but not those proximal the transition between the lateral portions 1104 and the cheek portions 1106.

In examples, the flap portion 1422 may be made of a resilient and flexible material—for example an elastomer such as silicone. In examples the flap portion 1422 may be integrally formed with the compliance portion 1150. In examples the flap portion 1422 may be secured to the compliance portion 1150.

FIG. 27A to FIG. 27J-2 show an exemplary interfacing structure 1100 according to an aspect of the present technology. In the example illustrated the lens connection portion is provided in the form of a chassis portion 1140 comprising a recessed lens receiving feature 1172 configured to receive a lens 1200 and locate the lens relative to the compliance portion 1150.

In this example, the chassis portion 1140 is made of the same material as the compliance portion 1150, such as an elastomer, but of a different durometer having greater stiffness. In examples the chassis portion 1140 may be permanently secured to the compliance portion 1150, for example by overmoulding. Referring to FIG. 27G to FIG. 27I-1, the chassis portion 1140 includes a recessed flange locating feature 1174 in which the compliance portion 1150 is located.

Referring to FIGS. 27J to 27J-2, the cover portion 1190 extends from an anterior position at the chassis portion 1140, over radially outward surfaces of the support flange 1154, to the radially outward cushion support flange portion 1161a. A gap is provided between the cover portion 1190 and the radially outward surfaces of the support flange 1154.

Referring to FIG. 27K, the chassis portion 1140 comprises an anterior cover locating feature 1176 configured to locate the cover portion 1190 at the anterior position. In this example the anterior cover locating feature 1176 comprises a first radially outward surface 1177a leading to a radially inwardly angled transition surface 1177b, and a posterior facing surface 1177c extending in a radially outward direction to a second radially outward surface 1177d, wherein the second radially outward 1177d is elevated beyond the first radially outward surface 1177a. The edge of the cover portion 1190 is received in the anterior cover locating feature 1176, with the angle of the transition surface 1177b assisting in preventing the edge being presented above the second radially outward surface 1177d.

Referring to FIG. 28A and FIG. 28B, in examples the radially outward cushion support flange portion 1161a comprises a cover support flange 1163 extending in an anterior direction. The cover support flange 1163 is provided in an intermediary position between the support flange 1154 and a distal end of the radially outward cushion support flange portion 1161a. In the example of FIG. 28A the cover portion 1190 is provided directly to the cover support flange 1163. In the example of FIG. 28B the cover portion 1190 is provided directly to the cover support flange 1163. In the example of FIG. 28B a cover support 1192 (for example, made of a foam material) is provided between the cover portion 1190 and the cover support flange 1163.

In aspects of the present technology the interfacing structure 1100 may be configured such that the lens 1200 is selectively removable. For example, the lens 1200 may include locating features which cooperate with features of the interfacing structure 1100 to hold the lens 1200 in place. FIG. 29 shows an exemplary interfacing structure 1100 according to an aspect of the present technology. In the example illustrated the chassis portion 1140 comprises a superior chassis portion 1142, and an inferior chassis portion 1144. In this example the superior chassis portion 1142 and inferior chassis portion 1144 are made of a non-elastomer thermoplastic, with a relatively high rigidity to assist with holding shape while having a relatively small cross-section.

In the example of FIG. 29 to FIG. 29-3, the interfacing structure 1100 is configured to allow resilient movement of the superior chassis portion 1142 relative to the inferior chassis portion 1144 in a superior-inferior direction. This arrangement assists with enabling selective assembly/disassembly of the lens 1200 with the interfacing structure 1100. In this example, the resiliency is provided by a chassis extension feature 1146 in the form of a portion of the compliance portion 1150 interconnecting the portion of the superior chassis portion 1142 and the inferior chassis portion 1144.

FIG. 30 shows an exemplary interfacing structure 1100 in which the chassis portion 1140 is made of a resilient material such as thermoplastic elastomer (TPE) or silicone having a relatively high degree of stiffness to assist with holding its shape. In this example, the chassis extension feature is provided in the form of a living hinge feature 1148 having a corrugated structure. The corrugated structure allows for elongation under force, allowing the chassis portion 1140 to be disengaged from the lens 1200.

FIG. 31A and FIG. 31B show an exemplary interfacing structure 1100 according to an aspect of the present technology. In this example, the lens connection portion 1152—i.e., the anterior perimeter that engages with the lens 1200—has a relatively high hardness to assist with maintaining shape and attaching to the lens 1200. In this example the forehead portion 1102 of the support flange 1154, and the transition into the lateral portions 1104, have relatively high hardness to provide compliance with a firmer feel on face at sides and forehead. Using higher hardness material enables thinner wall sections, which may assist with reducing weight and material cost. In this example the nasal portion 1108, the cheek portions 1106, and the transition into the lateral portions 1104, have a relatively low hardness and high elasticity to provide compliance and comfort with low resistance around the cheek and nose regions.

FIG. 32A, FIG. 32B and FIG. 32B-1 show an exemplary interfacing structure 1100 according to an aspect of the present technology. In this example the interfacing structure 1100 comprises a cover spacer feature 1194 configured to maintain a gap between the cover portion 1190 and the radially outward surfaces of the support flange 1154. In this example the cover spacer feature 1194 comprises a plurality of anterior-posterior spacer features 1195 extending between an anterior position and an posterior position along the support flange 1154, and projecting in a radially outward direction from the support flange 1154. The radially outward edge of the anterior-posterior spacer features 1195 provides a scaffold for the shape of the cover portion 1190. In examples the cover spacer feature 1194 comprises at least one lateral spacer feature 1196 between adjacent anterior-posterior spacer features 1195. In examples the cover spacer feature 1194 may be integral with the support flange 1154.

In examples the cover spacer feature 1194 may have a relatively high stiffness compared to the support flange 1154. Referring to FIG. 32C and FIG. 32D, the cover spacer feature 1194 may be made of the same material as the lens connection portion 1152.

FIG. 33 and FIG. 33-1 show an exemplary interfacing structure 1100 according to an aspect of the present technology. In this example, the cover portion 1190 is configured to maintain a non-linear shape, such that a gap is provided between the cover portion 1190 and the radially outward surfaces of the support flange 1154. In this example the cover portion 1190 comprises an outer cover 1197 (e.g., a textile layer), and a shape holding layer 1198 (e.g., a foam or other material that maintains its shape once formed). The shape holding layer 1198 provides a relatively stiff structure in comparison with the outer cover 1197 in order to achieve a desired shape, while allowing for compliance and folding characteristics to meet design needs.

FIG. 34 and FIG. 34-1 show an exemplary interfacing structure 1100 according to an aspect of the present technology. In this example the cover portion 1190 comprises an outer cover 1197 (e.g., a textile layer), and discrete cover rigidiser features 1199 (e.g., a foam or other material that maintains its shape once formed). The positioning of the cover rigidiser features 1199 is configured to provide greater stiffness to the outer cover 1197 in certain portions, while allowing flexure in the zones between the cover rigidiser features 1199.

FIG. 35 and FIG. 35-1 show an exemplary interfacing structure 1100 according to an aspect of the present technology. In this example the functionality of the support flange 1154 described with reference to other examples is provided by a cover portion 1190—for example as described with reference to FIG. 33 or FIG. 34.

### 5.2 Glossary

For the purposes of the present technology disclosure, in certain forms of the present technology, one or more of the following definitions may apply. In other forms of the present technology, alternative definitions may apply.

### 5.3 General

Ambient: In certain forms of the present technology, the term ambient will be taken to mean (i) external of the eyewear and/or user, and (ii) immediately surrounding the eyewear and/or user.

User: A person wearing the eyewear. For example, the person may be wearing, donning, and/or doffing the eyewear.

### 5.3.1 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a user interface in sealing relationship.

As an example, an I-beam may comprise a different bending stiffness (resistance to a bending load) in a first direction in comparison to a second, orthogonal direction. In another example, a structure or component may be floppy in a first direction and rigid in a second direction.

### 5.4 Materials

Closed-cell foam: Foam comprising cells that are completely encapsulated, i.e. closed cells.

Elastane: A polymer made from polyurethane.

Elastomer: A polymer that displays elastic properties. For example, silicone elastomer.

Ethylene-vinyl acetate (EVA): A copolymer of ethylene and vinyl acetate.

Fiber: A filament (mono or poly), a strand, a yarn, a thread or twine that is significantly longer than it is wide. A fiber may include animal-based material such as wool or silk, plant-based material such as linen and cotton, and synthetic material such as polyester and rayon. A fiber may specifically refer to a material that can be interwoven and/or interlaced (e.g., in a network) with other fibers of the same or different material.

Foam: Any material, for example polyurethane, having gas bubbles introduced during manufacture to produce a lightweight cellular form.

Neoprene: A synthetic rubber that is produced by polymerization of chloroprene. Neoprene is used in trade products: Breath-O-Prene.

Nylon: A synthetic polyamide that has elastic properties and can be used, for example, to form fibres/filaments for use in textiles.

Open-cell foam: Foam comprising cells, i.e. gas bubbles that aren't completely encapsulated, i.e. open cells.

Polycarbonate: a typically transparent thermoplastic polymer of Bisphenol-A Carbonate.

Polyethylene: A thermoplastic that is resistant to chemicals and moisture.

Polyurethane (PU): A plastic material made by copolymerizing an isocyanate and a polyhydric alcohol and, for example, can take the form of foam (polyurethane foam) and rubber (polyurethane rubber).

Semi-open foam: Foam comprising a combination of closed and open (encapsulated) cells.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Spacer Fabric: A composite construction comprised of two outer textile substrates joined together and kept apart by an intermediate layer of monofilaments.

Spandex: An elastic fibre or fabric, primarily comprised of polyurethane. Spandex is used in trade products: Lycra.

Textile: A material including at least one natural or artificial fiber. In this specification, a textile may refer to any material that is formed as a network of interwoven and/or interlaced fibers. A type of textile may include a fabric, which is constructed by interlacing the fibers using specific techniques. These include weaving, knitting, crocheting, knotting, tatting, tufting, or braiding. Cloth may be used synonymously with fabric, although may specifically refer to a processed piece of fabric. Other types of textiles may be constructed using bonding (chemical, mechanical, heat, etc.), felting, or other nonwoven processes. Textiles created through one of these processes are fabric-like, and may be considered synonymous with fabric for the purposes of this application.

Thermoplastic Elastomer (TPE): Are generally low modulus, flexible materials that can be stretched at room temperature with an ability to return to their approximate original length when stress is released. Trade products that use TPE include: Hytrel, Dynaflex, Medalist

Thermoplastic Polyurethane (TPU): A thermoplastic elastomer with a high durability and flexibility.

### 5.5 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

| 5.6 REFERENCE SIGNS LIST | |
|---|---|
| 100 | User |
| 110 | Foam spacer |
| 1000 | Goggles |
| 1100 | Interfacing structure |
| 1102 | Forehead portion |
| 1104 | Lateral portions |
| 1106 | Cheek portions |
| 1108 | Nasal portion |
| 1110 | Lateral nasal portions |
| 1111 | Posterior flaps |
| 1112 | Nose module |
| 1113 | Textile flaps |
| 1120 | Face engaging flange |
| 1130 | Cushion |
| 1132 | Contact layer |
| 1134 | First layer |
| 1136 | Second layer |
| 1138 | Nasal bridge portion |
| 1140 | Chassis portion |
| 1142 | Superior chassis portion |
| 1144 | Inferior chassis portion |
| 1146 | Chassis extension feature |
| 1148 | Living hinge feature |
| 1150 | Compliance portion |
| 1150a | First compliance portion |
| 1150b | Second compliance portion |
| 1150c | Third compliance portion |
| 1150d | Fourth compliance portion |
| 1151 | Anterior nasal flange portion |
| 1152 | Lens connection portion |
| 1153 | Anterior radially outward flange portion |
| 1154 | Support flange |
| 1155a | Anterior support flange portion |
| 1155b | Posterior support flange portion |
| 1156 | Thickened portion |
| 1158 | Flexure portion |
| 1159a | Nasal bridge contact portion |
| 1159b | Inferior portion |
| 1160 | Cushion support flange |
| 1161a | Radially outward cushion support flange portion |
| 1161b | Radially inward cushion support flange portion |
| 1162 | Cushion support flange angle |
| 1163 | Cover support flange |
| 1164 | Overmould feature |
| 1166 | Adhesive |
| 1168 | Mechanical interlock |
| 1170 | Carrier member |
| 1172 | Lens receiving feature |
| 1174 | Flange locating feature |
| 1176 | Anterior cover locating feature |
| 1177a | First radially outward surface |
| 1177b | Angled transition surface |
| 1177c | Posterior facing surface |
| 1177d | Second radially outward surface |
| 1180 | Hydrophilic vent portion |
| 1182 | Foam vent feature |
| 1184 | Reinforcement portions |
| 1190 | Cover portion |
| 1192 | Cover support |
| 1194 | Cover spacer feature |
| 1195 | Anterior-posterior spacer feature |
| 1196 | Lateral spacer feature |
| 1197 | Outer cover |
| 1198 | Shape holding layer |
| 1199 | Cover rigidiser |
| 1200 | Lens |
| 1202 | Superior lens edge |
| 1300 | Positioning and stabilising structure |
| 1302 | Integrated strap |
| 1304 | Strap connector |
| 1306 | Rigidiser |
| 1310 | Strap connection extension |
| 1312 | Strap connection feature |
| 1313 | Strap opening |
| 1314 | Pivot feature |
| 1316 | Strap connection receptacle |
| 1320 | Lateral strap portion |
| 1330 | Superior posterior strap portion |
| 1340 | Inferior posterior strap portion |
| 1350 | Adjustment buckles |
| 1400 | Lens frame |
| 1402 | Forehead portion |
| 1404 | Lateral portions |
| 1406 | Cheek portions |
| 1408 | Nasal portion |
| 1410 | Vents |
| 1420 | Vent valve |
| 1422 | Valve flap portion |
| 1424 | Vent valve spacer portion |
| 1426 | Secondary vent |
| 1500 | Adaptation portion |
| 1502 | Posterior nasal contact portion |
| 1503 | Anterior nasal contact portion |
| 1504 | Intermediary portion |
| 1506 | Lug portion |
| 1508 | Unconstrained lug portion |
| 1510 | Support member |
| 1512 | Bridging portion |
| 1514 | Lug locating features |
| 1516 | Bridge adaptation portion |
| 1518 | Lateral adaptation portion |
| 1520 | Transition adaptation portion |
| 1530 | Slot |
| 1532 | Pocket |
| 1534 | Reinforcing |
| 1600 | Helmet |
| 1602 | Helmet brim |

1. An interfacing structure connected, directly or indirectly, to a lens of eyewear, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient; and
a cushion configured to engage the user's face in use, the cushion provided to the cushion support flange.

2. The interfacing structure of claim 1, wherein the cushion is made of foam.

3. The interfacing structure of claim 2, wherein the cushion comprises a textile cover layer over the foam.

4. The interfacing structure of claim 1, wherein a nasal portion of the compliance portion comprises an adaptation portion between a lens connection portion and a posterior nasal contact portion of the compliance portion.

5. The interfacing structure of claim 4, wherein an anterior nasal contact portion of the compliance portion extends in an anterior direction from the adaptation portion.

6. The interfacing structure of claim 4, wherein the adaptation portion comprises a bridge adaptation portion extending along the lens connection portion over a bridge of the user's nose in use, and lateral adaptation portions extending from the bridge adaptation portion in a posterior direction towards a posterior edge of the posterior nasal contact portion.

7. The interfacing structure of claim 6, wherein the adaptation portion comprises a curved transition adaptation portion between the bridge adaptation portion and the lateral adaptation portions.

8. The interfacing structure of claim 6, wherein the bridge adaptation portion has a concave cross-section facing in the anterior-inferior direction, and the lateral adaptation portions have a concave cross-section facing in the posterior direction.

9. The interfacing structure of claim 1, wherein the cushion support flange comprises a radially outward cushion support flange portion extending radially outward from the support flange portion, and a radially inward cushion support flange portion extending radially inward from the support flange portion.

10. The interfacing structure of claim 1, wherein the support flange comprises a plurality of vents, and wherein the compliance portion further comprises a cover portion provided to radially outward surfaces of the support flange, covering the plurality of vents.

11. The interfacing structure of claim 10, wherein the cover portion is hydrophobic.

12. The interfacing structure of claim 10, wherein the cover portion extends between an anterior position, over the radially outward surfaces of the support flange, and a radially outward portion of the cushion support flange, such that a gap is provided between the cover portion and the radially outward surfaces of the support flange.

13. The interfacing structure of claim 1, further comprising a chassis portion configured to secure the lens relative to the compliance portion, the chassis portion being stiffer than the compliance portion, wherein the compliance portion is provided to the chassis portion.

14. The interfacing structure of claim 13, wherein the chassis portion comprises a superior chassis portion and an inferior chassis portion,
wherein the interfacing structure is configured to allow resilient movement of the superior chassis portion relative to the inferior chassis portion in a superior-inferior direction.

15. Eyewear, comprising:
a lens;
an interfacing structure connected, directly or indirectly, to the lens, the interfacing structure constructed and arranged to be in opposing relation with the user's face in use, the interfacing structure comprising:
a compliance portion comprising a support flange around a periphery of an eye region of the user's face, and a cushion support flange extending from the support flange, the support flange being flexible and resilient; and
a cushion configured to engage the user's face in use, the cushion provided to the cushion support flange.

16. The eyewear of claim 15, further comprising a positioning and stabilising structure configured to generate a force to hold the interfacing structure to the user's face in use.

17. The eyewear of claim 16, wherein at least a portion of the positioning and stabilising structure is integral with the interfacing structure.

18. The eyewear of claim 16, wherein the positioning and stabilising structure comprises:
a pair of lateral strap portions configured to be secured, directly or indirectly, to the lens and lion each side of the user's head in use;
a pair of rigidised portions connected to the pair of lateral strap portions;
a superior posterior strap portion configured to extend in a superior-posterior direction from the rigidised portions around the back of the user's head in use; and
an inferior posterior strap portion configured to extend in a posterior direction from the rigidised portions, inferior to the superior posterior strap portion, around the back of the user's head in use.
